SEQUENCE LISTING (59 sequences)

SEQ ID NO: 1 - protein, 118 aa, synthetic construct
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 2 - protein, 118 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 3 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 4 - protein, 116 aa, synthetic construct; identical to SEQ ID NO: 3

SEQ ID NO: 5 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYYGQGTLVTVSS

SEQ ID NO: 6 - protein, 116 aa, synthetic construct
QVQLVQSGAEVKKPGSSVKVSCKASGYTWTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 7 - protein, 111 aa, synthetic construct
MLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKR

SEQ ID NO: 8 - protein, 113 aa, synthetic construct
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 9 - protein, 111 aa, synthetic construct
DIQMTQSPSSLSASVGDRVTITCRASESVDNYGISFMNWFQQKPGGAPKLLIYAASMQGSGVPSRFSGSGSGTDFTLTISSLQPDDFATYYCQQSKEVPWTFGQGTKVEIK

SEQ ID NO: 10 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 11 - protein, 111 aa, synthetic construct
DIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIK

SEQ ID NO: 12 - protein, 111 aa, synthetic construct; identical to SEQ ID NO: 10

SEQ ID NO: 13 - protein, 18 aa, synthetic construct
GSTSGSGKPGSGEGSTKG

SEQ ID NO: 14 - protein, 247 aa, synthetic construct
QVQLVQSGAEVKKPGSSVKVSCKASGYTFTDYNMHWVRQAPGQGLEWIGYIYPYNGGTGYNCIKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKTSSG

SEQ ID NO: 15 - protein, 247 aa, synthetic construct
QVQLVQSGAEVKKPGSSVKVSCKASGYTFTDYNMHWVRQAPGQGLEWIGYIYPYNGGTGYNCIKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIKTSSG

SEQ ID NO: 16 - protein, 5 aa, synthetic construct
DYNMH

SEQ ID NO: 17 - protein, 19 aa, synthetic construct
YIYPYNGGTGYNQKFKSKA

SEQ ID NO: 18 - protein, 10 aa, synthetic construct
GRPAMDYWGQ

SEQ ID NO: 19 - protein, 15 aa, synthetic construct
RASESVDNYGISFMN

SEQ ID NO: 20 - protein, 7 aa, synthetic construct
AASNQGS

SEQ ID NO: 21 - protein, 9 aa, synthetic construct
QQSKEVPWT

SEQ ID NO: 22 - protein, 243 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKR

SEQ ID NO: 23 - protein, 8 aa, synthetic construct
GYTITDSN

SEQ ID NO: 24 - protein, 8 aa, synthetic construct
IYPYNGGT

SEQ ID NO: 25 - protein, 9 aa, synthetic construct
VNGNPWLAY

SEQ ID NO: 26 - protein, 10 aa, synthetic construct
ESLDNYGIRF

SEQ ID NO: 27 - protein, 9 aa, synthetic construct
QQTKEVPWS

SEQ ID NO: 28 - DNA, 65 bp, synthetic construct
  source 1..65: mol_type = other DNA
cccctccaccccacagtggggccactagggacaggattggtgacagaaaagccccatccttaggc

SEQ ID NO: 29 - DNA, 64 bp, synthetic construct
  source 1..64: mol_type = other DNA
cccctccaccccacagtggggccactagggacagattggtgacagaaaagccccatccttaggc

SEQ ID NO: 30 - DNA, 61 bp, synthetic construct
  source 1..61: mol_type = other DNA
cccctccaccccacagtggggccactagggaattggtgacagaaaagccccatccttaggc

SEQ ID NO: 31 - DNA, 44 bp, synthetic construct
  source 1..44: mol_type = other DNA
cccctccaccccacattggtgacagaaaagccccatccttaggc

SEQ ID NO: 32 - DNA, 56 bp, synthetic construct
  source 1..56: mol_type = other DNA
cccctccaccccacagtggggccacgattggtgacagaaaagccccatccttaggc

SEQ ID NO: 33 - DNA, 17 bp, synthetic construct
  source 1..17: mol_type = other DNA
cccctccaccccacagt

SEQ ID NO: 34 - DNA, 20 bp, synthetic construct
  source 1..20: mol_type = other DNA
ggggccactagggacaggat

SEQ ID NO: 35 - DNA, 22 bp, synthetic construct
  source 1..22: mol_type = other DNA
ttctcctgtggattcgggtcac

SEQ ID NO: 36 - DNA, 22 bp, synthetic construct
  source 1..22: mol_type = other DNA
ctctctggctccatcgtaagca

SEQ ID NO: 37 - DNA, 19 bp, synthetic construct
  source 1..19: mol_type = other DNA
tcctgggcaaacagcataa

SEQ ID NO: 38 - DNA, 20 bp, synthetic construct
  source 1..20: mol_type = other DNA
gagctgcagaaggacaagat

SEQ ID NO: 39 - DNA, 21 bp, synthetic construct
  source 1..21: mol_type = other DNA
ctcgtgtcatctggatgtctg

SEQ ID NO: 40 - DNA, 22 bp, synthetic construct
  source 1..22: mol_type = other DNA
ctttgagctctactggcttctg

SEQ ID NO: 41 - DNA, 18 bp, synthetic construct
  source 1..18: mol_type = other DNA
gcgagtgaagacggcatg

SEQ ID NO: 42 - DNA, 20 bp, synthetic construct
  source 1..20: mol_type = other DNA
gtctgtgctagctcttccag

SEQ ID NO: 43 - DNA, 19 bp, synthetic construct
  source 1..19: mol_type = other DNA
gcgatgtcagaagggtaaa

SEQ ID NO: 44 - DNA, 20 bp, synthetic construct
  source 1..20: mol_type = other DNA
ggcggacactctgactacat

SEQ ID NO: 45 - DNA, 23 bp, synthetic construct
  source 1..23: mol_type = other DNA
ggggccactagggacaggattgg

SEQ ID NO: 46 - DNA, 14 bp, synthetic construct
  source 1..14: mol_type = other DNA
gcggccggcgcgcc

SEQ ID NO: 47 - DNA, 804 bp, synthetic construct
  source 1..804: mol_type = other DNA
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 48 - DNA, 23 bp, synthetic construct
  source 1..23: mol_type = other DNA
cactagggacagcgatcgggtac

SEQ ID NO: 49 - DNA, 39 bp, synthetic construct
  source 1..39: mol_type = other DNA
atcgatcgcaggcgcaatcttcgcatttcttttttccag

SEQ ID NO: 50 - DNA, 540 bp, synthetic construct
  source 1..540: mol_type = other DNA
atcgatcacgagactagcctcgagaagcttgatatcgaattccacggggttggacgcgtcttaattaaggatccaaggtcaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcacc

SEQ ID NO: 51 - DNA, 708 bp, synthetic construct
  source 1..708: mol_type = other DNA
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 52 - DNA, 2034 bp, synthetic construct
  source 1..2034: mol_type = other DNA
atgctgctgctggtgacctctctgctgctgtgcgagctgccacacccagccttcctgctgatcccagacatccagatgacacagagccccagctccctgagcgcctccgtgggcgacagagtgaccatcacatgtagggcctctgagagcgtggataactatggcatcagcttcatgaattggtttcagcagaagcctggcggcgccccaaagctgctgatctacgcagccagcatgcagggctccggcgtgccctctcggttctccggctctggcagcggcaccgacttcaccctgacaatctctagcctgcagccagacgatttcgccacatactattgccagcagagcaaggaggtgccctggacctttggccagggcacaaaggtggagatcaagggctccacctctggcagcggcaagcctggcagcggagagggctccacaaagggacaggtgcagctggtgcagtccggagccgaggtgaagaagccaggctcctctgtgaaggtgtcttgtaaggccagcggctataccttcacagactacaacatgcactgggtgcgccaggcaccaggacagggcctggagtggatcggctacatctatccttacaacggcggcaccggctataatcagaagtttaagtccaaggccaccatcacagccgatgagtctaccaatacagcctacatggagctgagcagcctgcggtccgaggacacagccgtgtactattgcgcccggggcagacccgctatggactattggggccagggcaccctggtgacagtgtctagcgagagcaagtacggaccaccttgcccaccatgtcctgcaccagagttcctgggaggaccttccgtgttcctgtttcctccaaagccaaaggacaccctgatgatcagccggaccccagaggtgacatgcgtggtggtggacgtgagccaggaggaccccgaggtgcagttcaactggtacgtggatggcgtggaggtgcacaatgccaagaccaagccaagagaggagcagtttaactccacctatagggtggtgtctgtgctgacagtgctgcaccaggactggctgaacggcaaggagtacaagtgcaaggtgtccaataagggcctgccttcctctatcgagaagaccatctctaaggcaaagggacagccaagggagccacaggtgtatacactgccccctagccaggaggagatgaccaagaaccaggtgtccctgacatgtctggtgaagggcttttacccttctgacatcgccgtggagtgggagagcaatggccagccagagaacaattataagaccacaccacccgtgctggactctgatggcagcttctttctgtacagccgcctgaccgtggataagtcccggtggcaggagggcaacgtgttctcctgctctgtgatgcacgaggccctgcacaatcactacacacagaagagcctgtccctgtctctgggcaagatgttttgggtgctggtggtggtgggaggcgtgctggcctgttattccctgctggtgaccgtggccttcatcatcttttgggtgcgctccaagcggagccggggcggacactctgactacatgaacatgaccccacggagacccggacctacaaggaagcactatcagccctacgcccctccacgggacttcgcagcatatcgcagccgggtgaagtttagcagatccgccgatgcaccagcatatcagcagggacagaatcagctgtacaacgagctgaatctgggcaggcgcgaggagtacgacgtgctggataagaggcggggccgggaccccgagatgggaggcaagcccaggcgcaagaaccctcaggagggcctgtataatgagctgcagaaggacaagatggccgaggcctacagcgagatcggcatgaagggagagcggagaaggggcaagggacacgatggcctgtatcagggcctgtccaccgccacaaaggacacctacgatgcactgcacatgcaggccctgccacctcggtga

SEQ ID NO: 53 - DNA, 2331 bp, synthetic construct
  source 1..2331: mol_type = other DNA
atgctgctgctggtgacctccctgctgctgtgcgagctgccacaccctgcctttctgctgatcccagacatccagatgacacagagccccagctccctgtctgccagcgtgggcgacagagtgaccatcacatgtagggcctccgagtctgtggataactatggcatcagctttatgaattggttccagcagaagccaggaggcgcccctaagctgctgatctacgcagcctccatgcagggctctggcgtgcccagccgctttagcggctccggctctggcaccgatttcaccctgacaatctctagcctgcagccagacgattttgccacatactattgccagcagtccaaggaggtgccctggaccttcggccagggcacaaaggtggagatcaagggcagcacctccggctctggcaagcctggctccggagagggctctacaaagggacaggtgcagctggtgcagagcggagccgaggtgaagaagccaggctcctctgtgaaggtgagctgtaaggcctccggctatacctttacagactacaacatgcactgggtgagacaggcaccaggacagggcctggagtggatcggctacatctatccttacaacggcggcaccggctataatcagaagttcaagagcaaggccaccatcacagccgatgagtccaccaatacagcctacatggagctgagcagcctgaggagcgaggacacagccgtgtactattgcgccagaggcaggcctgctatggactattggggccagggcaccctggtgacagtgtctagcgagtccaagtacggaccaccttgcccaccatgtccagcaccagagtttctgggaggacctagcgtgtttctgttccctccaaagccaaaggacaccctgatgatcagcagaacccccgaggtgacatgcgtggtggtggacgtgtcccaggaggaccccgaggtgcagtttaactggtacgtggatggcgtggaggtgcacaatgccaagaccaagcctagagaggagcagttcaactccacctatagggtggtgtctgtgctgacagtgctgcaccaggactggctgaacggcaaggagtacaagtgcaaggtgtctaataagggcctgccatcctctatcgagaagaccatcagcaaggccaagggccagcctagggagccacaggtgtatacactgcccccttcccaggaggagatgaccaagaaccaggtgtctctgacatgtctggtgaagggcttctacccatccgacatcgccgtggagtgggagtctaatggccagcccgagaacaattataagaccacaccacccgtgctggactctgatggcagcttctttctgtactctcgcctgaccgtggataagagccggtggcaggagggcaacgtgtttagctgctccgtgatgcacgaggccctgcacaatcactacacacagaagtctctgagcctgtccctgggcaagagcaacctgttcgtggcctcctggatcgccgtgatgatcatctttcgcatcggcatggccgtggccatcttctgctgtttctttttcccatccggaggctctggaggaggctccggctggcggagaaagcggaaggagaagcagagcgagacctcccctaaggagtttctgacaatctatgaggacgtgaaggatctgaagaccaggcgcaatcacgagcaggagcagaccttcccaggaggaggctctacaatctacagcatgatccagtcccagagcagcgccccaaccagccaggagccagcctatacactgtactctctgatccagcctagccggaagtctggcagccgcaagcggaaccactccccatctttcaattctaccatctatgaagtgatcggcaagagccagcctaaggcccagaacccagccagactgtccaggaaggagctggagaattttgacgtgtactctggaggcagcggaggaggctctggccgcgtgaagttcagccggtccgccgatgccccagcctataagcagggccagaaccagctgtacaacgagctgaatctgggccggagagaggagtacgacgtgctggataagaggcggggccgggaccccgagatgggaggcaagccccggagaaagaaccctcaggagggcctgtataatgagctgcagaaggacaagatggccgaggcctactccgagatcggcatgaagggagagaggcgccggggcaagggacacgatggcctgtatcagggcctgagcaccgccacaaaggacacctacgatgccctgcacatgcaggccctgcctccacggtgatga

SEQ ID NO: 54 - DNA, 147 bp, synthetic construct
  source 1..147: mol_type = other DNA
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattc

SEQ ID NO: 55 - DNA, 1000 bp, synthetic construct
  source 1..1000: mol_type = other DNA
gattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttgg

SEQ ID NO: 56 - DNA, 235 bp, synthetic construct
  source 1..235: mol_type = other DNA
ggcatggggttgggtgagggaggagagatgcccggagaggacccagacacggggaggatccgctcagaggacatcacgtggtgcagcggcgcgccggccgcagaaagggagtagaggcggccacgacctggtgaacacctaggacgcaccattctcacaaagggagttttccacacggacacccccctcctcaccacagccctgccaggacggggctggctactggccttatctc

SEQ ID NO: 57 - DNA, 243 bp, synthetic construct
  source 1..243: mol_type = other DNA
gcgagtgaagacggcatggggttgggtgagggaggagagatgcccggagaggacccagacacggggaggatccgctcagaggacatcacgtggtgcagcggcgcgccggccgcaggaagggagtagaggcggccacgacctggtgaacacctaggacgcaccattctcacaaagggagttttccacacggacacccccctcctcaccacagccctgccaggacggggctggctactggcctta

SEQ ID NO: 58 - DNA, 229 bp, synthetic construct
  source 1..229: mol_type = other DNA
gcgagtgaagacggcatggggttgggtgagggaggagagatgcccggagaggacccagacacggggaggatccgctcagaggacatcacgtggtgcagcggcgcgcagagagggagtggccaactccatcactaggggttcctgcggccgcagaaagggagtagaggcggccacgacctggtgaacacctaggacgcaccattctcacaaagggagttttccacacgga

SEQ ID NO: 59 - DNA, 234 bp, synthetic construct
  source 1..234: mol_type = other DNA
gcgagtgaagacggcatggggttgggtgagggaggagagatgcccggagaggacccagacacggggaggatccgctcagaggacatcacgtggtgcagcggccgcagaaagggagtagaggcggccacgacctggtgaacacctaggacgcaccattctcacaaagggagttttccacacggacacccccctcctcaccacagccctgccaggacggggctggctactggcctt

(19) United States
(12) Patent Application Publication
(10) Pub. No.: US 2026/0297189 A1
(43) Pub. Date: Oct. 1, 2026

(54) CD-33 PROTEIN-BINDING COMPOSITIONS AND METHODS RELATED THERETO

(71) Applicant: RESEARCH INSTITUTE AT NATIONWIDE CHILDREN'S HOSPITAL, Columbus, OH (US)

(72) Inventors: Dean Anthony LEE, Canal Winchester, OH (US); Meisam NAEIMI KARAROUDI, Columbus, OH (US)

(21) Appl. No.: 19/489,060

(22) PCT Filed: May 31, 2024
(86) PCT No.: PCT/US2024/032094

§ 371 Date: Dec. 1, 2025

Related U.S. Application Data
(60) Provisional application No. 63/505,550, filed on Jun. 1, 2023.

(51) Int. Cl. C07K 16/28; A61K 40/15; A61K 40/31; A61K 40/42; A61P 35/02; C12N 5/0783

(52) U.S. Cl. CPC C07K 16/2803; A61K 40/15; A61K 40/31; A61K 40/421; A61P 35/02; C12N 5/0646; C07K 2317/565; C07K 2317/622; C07K 2319/02; C07K 2319/03; C12N 2510/00

(57) ABSTRACT

Disclosed are single-chain variable fragments, chimeric antigen receptors, and methods to treat and prevent CD33-associated pathologies, such as hematologic cancers and neurological disorders.

### CROSS-REFERENCE TO RELATED APPLICATION

This PCT application claims priority to, and the benefit of, U.S. Provisional Patent Application No. 63/505,550, filed Jun. 1, 2023, entitled “CD-33 PROTEIN-BINDING COMPOSITIONS AND METHODS RELATED THERETO,” which is incorporated by reference herein in its entirety.

### REFERENCE TO SEQUENCE LISTING

The sequence listing submitted on May 31, 2024, as an .XML file entitled “10935-021WO1” created on May 31, 2024, and having a file size of 65,536 bytes is hereby incorporated by reference pursuant to 37 C.F.R. § 1.52 (e) (5).

### FIELD

The present disclosure relates CD33 protein-binding compositions, nucleic acid encoding CD33 protein binding compositions, and methods of use thereof.

### BACKGROUND

CD33 is a transmembrane protein that is found on cells of myeloid lineage. Also known as Siglec-3, sialic acid binding Ig-like 3, SIGLEC3, gp67, and p67. CD33 is expressed on all normal myeloid cells downstream of the common myeloid progenitor and is used as both a diagnostic marker and a therapeutic target for AML. Antibody-drug conjugates have been developed to treat acute myelogenous leukemia based on this expression differential. The Siglec protein family is associated with multiple human disease including, autoimmunity, susceptibility to infection, multiple types of cancer including lymphoma, leukemia and acute myeloid leukemia, systemic lupus erythematosus, rheumatoid arthritis, neurodegenerative disorders, asthma, allergy, sepsis, chronic obstructive pulmonary disease, graft-versus-host disease, eosinophilia, and osteoporosis (Macauley S M. et al., (2014) Nature Reviews Immunology 14, 653-666).

The field continues to develop, with some researchers disclosing use of single chain variable fragments of the murine 195 antibody (m195) or humanized 195 antibody (hu195) having the known N-terminus to C-terminus structure. Treatments of CD33-associated disease states are limited, and additional treatments are needed.

The compositions and methods disclosed herein address these needs and more.

### SUMMARY

The present disclosure provides nucleic acids, expression vectors, cells, and compositions thereof comprising an scFv for treating pathologies related to or caused by CD33.

In some aspects, disclosed herein is a composition comprising a single-chain variable fragment (scFv) comprising a heavy chain variable region (VH), a light chain variable region (VL), and an acceptable carrier, wherein the VH comprises one, two, or three heavy chain complementarity determining regions (CDRHs), and the VL comprises one, two, or three light chain complementarity determining regions (CDRLs), and wherein the CDRHs or the CDRLs comprise 70%, 75%, 80%, 85%, 90%, 95%, 99%, or 100% sequence identity to SEQ ID NO: 16, SEQ ID NO: 17, SEQ ID NO: 18, SEQ ID NO: 19, SEQ ID NO: 20, or SEQ ID NO: 21.

In some embodiments, the scFv comprises 70%, 75%, 80%, 85%, 90%, 95%, 99%, or 100% sequence identity to SEQ ID NO: 52, SEQ ID NO: 53, or a variant thereof. In some embodiments, the VH comprises 70%, 75%, 80%, 85%, 90%, 95%, 99%, or 100% sequence identity to SEQ ID NO: 1, SEQ ID NO: 2, SEQ ID NO: 3, SEQ ID NO: 4, SEQ ID NO: 5 or SEQ ID NO: 6, and the VL comprises 70%, 75%, 80%, 85%, 90%, 95%, 99%, or 100% sequence identity to SEQ ID NO: 7, SEQ ID NO: 8, SEQ ID NO: 9, SEQ ID NO: 10, SEQ ID NO: 11 or SEQ ID NO: 12.

In some embodiments, the composition further comprises a linker attached to the VH and the VL, wherein the linker comprises 80%, 85%, 90%, 95%, 99%, or 100% sequence identity to SEQ ID NO: 13. In some embodiments, the composition further comprises 80%, 85%, 90%, 95%, 99%, or 100% sequence identity to SEQ ID NO: 23, SEQ ID NO: 24, SEQ ID NO: 25, SEQ ID NO: 26, SEQ ID NO: 27, or SEQ ID NO: 28.

In some embodiments, the composition of any preceding aspect comprises a chimeric antigen receptor (CAR).

In some aspects, disclosed herein is a nucleic acid encoding the scFv of any preceding aspect.

In some aspects, disclosed herein is an expression vector comprising a nucleic acid encoding the scFv of any preceding aspect.

In some aspects, disclosed herein is a cell comprising the composition or the expression vector of any preceding aspect.

In some embodiments, the cell of any preceding aspect includes, but is not limited to a T-cell, a natural killer (NK) cell, a genetically-modified T-cell, or a genetically-modified NK cell.

In some aspects, disclosed herein is a method of treating, preventing, ameliorating, reducing, and/or decreasing a CD33-associated pathology (including, but not limited to a hematologic cancer and a neurodegenerative disorder) in a subject in need thereof, the method comprising administering to the subject a therapeutically effective amount of a composition comprising a single-chain variable fragment (scFv), wherein the scFv comprises a heavy chain variable region (VH), a light chain variable region (VL), and an acceptable carrier, wherein the VH comprises one, two, or three heavy chain complementarity determining regions (CDRHs), and the VL comprises one, two, or three light chain complementarity determining regions (CDRLs), and wherein the CDRHs or the CDRLs comprise at least 80% sequence identity to SEQ ID NO: 16, SEQ ID NO: 17, SEQ ID NO: 18, SEQ ID NO: 19, SEQ ID NO: 20, or SEQ ID NO: 21.

In some embodiments, the method of treating, preventing, ameliorating, reducing, and/or decreasing comprises the scFv comprising 70%, 75%, 80%, 85%, 90%, 95%, 99%, or 100% sequence identity to SEQ ID NO: 52, SEQ ID NO: 53, or a variant thereof. In some embodiments, the method of treating, preventing, ameliorating, reducing, and/or decreasing comprises the VH comprising 70%, 75%, 80%, 85%, 90%, 95%, 99%, or 100% sequence identity to SEQ ID NO: 1, SEQ ID NO: 2, SEQ ID NO: 3, SEQ ID NO: 4, SEQ ID NO: 5 or SEQ ID NO: 6, and the VL comprises 60%, 65%, 70%, 75%, 80%, 85%, 90%, 95%, 99%, or 100% sequence identity to SEQ ID NO: 7, SEQ ID NO: 8, SEQ ID NO: 9, SEQ ID NO: 10, SEQ ID NO: 11 or SEQ ID NO: 12.

In some embodiments, the method of treating, preventing, ameliorating, reducing, and/or decreasing further comprises a linker attached to the VH and the VL, wherein the linker comprises 80%, 85%, 90%, 95%, 99%, or 100% sequence identity to SEQ ID NO: 13. In some embodiments, the method of treating, preventing, ameliorating, reducing, and/or decreasing further comprises at least 80% sequence identity to SEQ ID NO: 23, SEQ ID NO: 24, SEQ ID NO: 25, SEQ ID NO: 26, SEQ ID NO: 27, or SEQ ID NO: 28.

In some embodiments, the method of treating, preventing, ameliorating, reducing, and/or decreasing comprises the composition comprising a chimeric antigen receptor (CAR).

In some embodiments, the method treats a hematologic cancer (such as, for example acute myelogenous leukemia), neurodegenerative disorder (such as, for example Alzheimer's disease), lymphoma, leukemia, systemic lupus erythematosus, rheumatoid arthritis, asthma, allergy, sepsis, chronic obstructive pulmonary disease, graft-versus-host disease (GVHD), eosinophilia, or osteoporosis).

### BRIEF DESCRIPTION OF FIGURES

The accompanying figures, which are incorporated in and constitute a part of this specification, illustrate several aspects described below.

FIG. 1 shows exemplary flow data of the CD33-CAR expression using whitlow linker antibody.

FIG. 2 shows the component make up of GEN2, V1, V2, V3, and V4 CAR T cells including the scFv, transmembrane stalk, S2 signaling domain, S1 signaling domain and integration site.

FIG. 3 shows the absolute cell counts for cytotoxicity pre-stimulation and 48 hours post-transduction at the stated effector:target ratios using flow cytometry.

FIG. 4 shows side scatter and CD33 expression 5 days post transduction for WT, GEN2, V1, and V2 CAR T cells. . . .

FIG. 5 shows the absolute cell counts for cytotoxicity pre-stimulation and 9 days post-transduction at the stated effector:target ratios using flow cytometry.

FIGS. 6A, 6B, 6C, and 6D show the shows side scatter and CD33 expression 9 days post-transduction for WT (6A), GEN2 (6B), V1 (6C), and V2 (6D) CAR T cells

FIGS. 7A and 7B show the absolute cell counts of Kasumi-1 cells 15-days post transduction and at 24 hours (7A) and 48 hours (7B) cytotoxicity using flow cytometry.

FIG. 8 shows the percentage specific lysis of Kasumi-1 cells 15 days post-transduction and after 24 hours cytotoxicity by flow cytometry at the stated effector:target ratios.

FIG. 9 shows the percentage specific lysis of Kasumi-1 cells 15 days post-transduction and after 48 hours cytotoxicity by flow cytometry at the stated effector:target ratios.

FIGS. 10A, 10B, 10C, and 10D show the presence of CD33 CAR T cells 15 days post-transduction with WT (10A), GEN2 (10B), V1 (10C), or V2 (10D) CAR T cells.

FIG. 11 shows the CD33-CAR expression relative to V1 for GEN2, V1, and V2 CAR T cells.

FIGS. 12A, 12B, 12C, and 12D show the expression of Eotaxin (12A), FGF (12B), G-CSF (12C), and GM-CSF (12D) following 24 hours and 48 hours stimulation for WT, GEN2, V1, and V2 CAR T cells.

FIG. 13 shows the percentage specific lysis of Kasumi-1 cells after 4 hours cytotoxicity by flow cytometry at the stated effector:target ratios.

FIG. 14 shows the percentage specific lysis of Kasumi-1 cells after 48 hours cytotoxicity by flow cytometry at the stated effector:target ratios.

FIGS. 15A, 15B, 15C, 15D, 15E, 15F, and 15G show CD33 expression for V1 (AACS1 integration site) CAR T cells (15A), GEN2 (AACS1 integration site) CAR T cells (15B), V3 (CD38 integration site) CAR T cells (15C), V1 (CD38 integration site) CAR T cells (15D), V2 (CD38 integration site) CAR T cells (15E), V4 (CD38 integration site) CAR T cells (15F), and WT T cells (15G).

FIG. 16 shows the absolute cell counts of Kasumi-1 cells at 4 hours cytotoxicity using countbrite beads at the state effector:target ratio.

FIG. 17 shows the percentage specific lysis of Kasumi-1 cells at 4 hours cytotoxicity using countbrite beads at the state effector:target ratio.

FIG. 18 shows the absolute cell counts of Kasumi-1 cells at 48 hours cytotoxicity using countbrite beads at the state effector:target ratio.

FIG. 19 shows the percentage specific lysis of Kasumi-1 cells at 48 hours cytotoxicity using countbrite beads at the state effector:target ratio.

FIG. 20 shows the absolute cell counts of AML-10 cells at 4 hours cytotoxicity using countbrite beads at the state effector:target ratio.

FIG. 21 shows the percentage specific lysis of AML-10 cells at 4 hours cytotoxicity using countbrite beads at the state effector:target ratio.

FIG. 22 shows the absolute cell counts of AML-10 cells at 48 hours cytotoxicity using countbrite beads at the state effector:target ratio.

FIG. 23 shows the percentage specific lysis of AML-10 cells at 48 hours cytotoxicity using countbrite beads at the state effector:target ratio.

FIGS. 24A, 24B, 24C, 24D, 24E, 24F, and 24G show the CD33 expression day 2 post transduction for V1 (AACS1 integration site) CAR T cells (24A), GEN2 (AACS1 integration site) CAR T cells (24B), V3 (CD38 integration site) CAR T cells (24C), V1 (CD38 integration site) CAR T cells (24D), V2 (CD38 integration site) CAR T cells (24E), V4 (CD38 integration site) CAR T cells (24F), and WT T cells (24G).

FIG. 25 shows the CD33 CAR expression of GEN2 (AAVS1 integration site), V1 (AAVS1 integration site), V1 (C38 integration site), V2 GEN2 (CD38 integration site), V3 (CD38 integration site), and V4 (C38 integration site) CAR T cells relative to V1.

FIG. 26 shows a graphical representation of an efficient method using CRISPR and AAV for site-directed gene knock-in into human primary natural killer (NK) cells, generation of CD33-specific CAR-NK cells and the efficacy against AML.

FIGS. 27A, 27B, and 27C show the efficient CRISPR targeting of AAVS1 in FC21-expanded human primary NK cells. FIG. 27A shows the relative gene expression level of NHEJ- and HR-related genes in native and FC21-expanded NK cells (n=4). FIG. 27B shows the ATAC-seq data shows that AAVS has a similar chromatin accessibility between freshly isolated (naïve) and FC21-expanded NK cells (n=2). FIG. 27C shows the efficacy of Cas9/RNP-mediated targeting of AAVS1 in NK cells. Reference genome sequence for AAVS1 is shown in the top row, indicating the crRNA target sequence and the PAM sequence. The mutated sequences that were identified, and their associated frequencies, are shown in the bottom rows. Data are shown as mean±SD. Significant p values are indicated from individual ratio paired t tests and are not adjusted for multiple comparisons.

FIGS. 28A, 28B, and 28C show the constructs, integration process, and method workflow for gene insertion through HR and CRISPaint. FIG. 28A shows the constructs and integration process through HR. Cas9/RNP introduces a DSB in AAVS1, after which DNA encoding a gene of interest can be integrated through HR by varying lengths of HAs. The schematics show the construct designs for integration of DNA encoding mCherry with HAs between 30 and 1000 base pairs (bps) for Cas0-targeting site in AAVS1 and cloned in ssAAV6 and/or scAAV6 backbone. FIG. 28B shows the constructed design for insertion of DNA encoding mCherry through CRISPaint and cloned in scAAV (top). The schematics showing the integration process for CRISPaint gene insertion through homology-independent DNA repair pathway. FIG. 28C shows the schematics of workflow to electroporate Cas9/RNP and transduce AAV6 for gene delivery into NK cells.

FIGS. 29A, 29B, and 29C shows the combination of AAV6 and Cas9/RNP results in efficient generation of mCherry-expressing NK cells. FIG. 29A shows the representative flow cytometry of human primary NK cells expressing mCherry 2 days after Cas9/RNP electroporation and AAV6 transduction (MOI=3.5×10⁵). FIG. 29B shows the efficiency of Cas9/RNP and AAV6-mediated mCherry expression in human primary NK cells through HR and CRISPaint (n=3). Data are shown as mean±SD. FIG. 29C shows the mCherry expression in NK cells after enrichment and 2 additional weeks of expansion.

FIGS. 30A, 30B, 30C, 30D, 30E, and 30F show the successful generation of CD33CAR-expressing NK cells using combination of Cas9/RNP and AAV6. FIG. 30A shows the schematic of anti-CD33 CAR constructs (Gen2 and Gen4v2) with HAs for AAVS1-targeting site and cloned in ssAAV. FIG. 30B show the schematic of CAR protein structural design. FIG. 30C shows the representative flow cytometry showing the expression of CD33CAR on NK cells 7 days after Cas9/RNP electroporation and AAV6 transduction (3×10⁵). FIG. 30D shows the mean fluorescence intensity (MFI) of CD33CAR expression for Gen2 versus Gen4v2 (p=0.0014, unpaired t test). FIG. 30E shows the CD33CAR expression level on NK cells 7 and 14 days after transduction and electroporation (n=3, n.s. from two-way ANOVA adjusted for multiple comparisons). FIG. 30F shows the fold expansion of CD33CAR-expressing NK cells on feeder cells for 14 days starting from 3×10⁵ cells (n=3, n.s. from one-way ANOVA adjusted for multiple comparisons). Data are shown as mean±SD. Significant p values indicated are from individual ratio t tests and are not adjusted for multiple comparisons. **p<0.01.

FIGS. 31A, 31B, and 31C show the integration of the transgene in AAVS1 locus confirmed by PCR and TLA. FIG. 31A shows the schematic of PCR primers designed inside and outside of CD33CARs encoding DNA and integrated in AAVS1. FIG. 31B shows that amplicons were amplified and visualized on 1% agar gel from NK cells to specifically identify the CD33CAR gene inserted at the AAVS1 locus outside of the trans [primers forward-1 and reverse-1] and PCR condition 2 [primers forward-2 and reverse-2], and confirmed by amplifying the AAVS1 locus outside of the transgenes (PCR condition 3 [primers forward-2 and reverse-1]). FIG. 31C shows the TLA sequence coverage across the human genome in CAR-NK cells expanded for 14 days using primers designed to detect integration of CD33CAR-Gen2. The chromosomes are indicated on the y axis and the chromosomal position on the x axis. A single integration site was identified above background, as shown in circle.

FIGS. 32A, 32B, 32C, 32D, 32E, 32F, and 32G show that the CD33CAR NK cells have enhanced anti-AML activity. FIGS. 32A and 32B show that CD33CAR NK cells degranulate significantly higher than wild-type NK cells when co-cultured with Kasumi-1 (FIG. 32A) and HL60 (FIG. 32B) cells. FIG. 32C shows the expressing CD33CAR on NK cells also enhances overall antitumor activity ok NK cells against Kasumi-1 in three donors. FIGS. 32D and 32E show the enhanced cytotoxicity activity was also observed against AML-10 primary cells (FIG. 32D) but not against HL-60 cells (FIG. 32E). FIG. 32F shows the superior antitumor activity of the CD33CAR-Gen2 was also observed in a long-term cytotoxicity assay using xCelligence against Kasumi. FIG. 32G shows the cytometry by time of flight (cyTOF) analysis showed killing by the CD33CAR Gen2 NK cells of specific subsets of AML that were resistant to wild-type NK cells. *p<0.05, **p<0.01, ***p<0.001, or ****p<0.0001, by two-way ANOVA adjusted for multiple comparisons.

FIGS. 33A and 33B show that targeting AAVS1 in expanded CD3^{negative}CD56^{positive} NK cells does not alter normal function of the cells. FIG. 33A shows a schematic of workflow for electroporation of Cas9/RNP into day 7 expanded human primary NK cells to target AAVS1. FIG. 33B shows a cytotoxicity assay of AAVS1^{KO} NK cells does not show any suppression in their antitumor activity against AML cell line.

FIGS. 34A, 34B, and 34C show a representative flow analysis of mCherry and CD33CAR NK cells expression in freshly isolated and expanded NK cells. FIG. 34A shows a representative flow cytometry analysis of mCherry expression level in freshly isolated NK cells electroporated with Cas9/RNP and transduced with AAV6. FIGS. 34B and 34C shows representative flow cytometry analyses of CD33CAR expression level 7 days (FIG. 34B) and 14 days (FIG. 34C) post electroporation and AAV6 transduction in human NK cells.

FIGS. 35A and 35B show a representative flow cytometry of CD33CAR expressing NK cells transduced with different MOIs. FIG. 35A shows the analysis of CD33CAR-Gen2 expression level in NK cells transduced with 10K-300K MOI of ssAAV6 encoding CD33CAR-Gen2 showed successful expression of CAR on NK cells isolated from three healthy donors (FIG. 35B).

FIGS. 36A and 36B show the TLA analysis shows the integration of the transgene. FIG. 36A shows the TLA sequence coverage (in grey) across the vector integration locus, human chr19: 54,550,476-55,682,266. The blue arrow indicates the location of the breakpoint sequences. Y-axes are limited to 20× and 100× resp. The coverage profile this figure shows that no genomic rearrangements have occurred in the region of the integration site. From this data it is concluded that the vector has integrated as intended in human chromosome chr19: 55,115,754-55,115,767. According to the RefSeq this is in intron 1 of PPP1R12C. Other integration sites were observed between chr19: 55,115,155-55,116,371. According to the RefSeq this is also in intron 1 of PPP1R12C. FIG. 36B shows the NGS sequencing coverage (in grey) across the vector. Black arrows indicate the primer location. The blue arrows indicate the locations of the identified vector-genome breakpoint sequences (described below). The vector map is shown on the bottom. Y-axes are limited to 100×. High coverage is observed across the region between the ITR sites, vector sequence Vector: 12-4,255. Low/no coverage is observed across the Vector: 0-11 and 4,256-6, 864 indicating the backbone has not integrated in a large proportion of this sample, potentially a small subset of the sample might contain the backbone as well. Also, coverage is observed at the ITRs, indicating that next to the integration through the homology arms ITR based integrations also occurred in the sample. Sequence variants and structural variants were called in the covered regions. Sequence variants A single sequence variants of the transgene was detected by this method as shown in Supplementary Table 4. The frequency of detection suggests this variant was present within the AAV6 vector itself.

FIGS. 37A, 37B, 37C, 37D, 37E, and 37F show that the CD33CAR NK cells have enhanced anti-AML activity. FIG. 37A shows the expression level of CD33 on different AML cell lines. FIGS. 37B, 37C, and 37D show the representative single experiments presented in the cumulative data shown in FIG. 32. CD33CAR NK cells kill significantly higher than wildtype NK cells when cocultured with Kasumi-1 (FIG. 37B), and HL60 (FIG. 37C), and primary AML10 cells (FIG. 37D). The xCelligence assay showed a significantly enhanced cytotoxic activity of CD33CAR NK cells against Kasumi-1 in different time-points and E:T ratios (FIG. 37E). **** adjusted P value <0.0001. CD33CAR-Gen NK cells also secreted significantly higher TNF-alpha (TNF-α) and IFN-gamma (IFN-γ) when cocultured with Kasumi-1 (FIG. 37F). * adjusted P value=0.01, **** adjusted P value <0.0001.

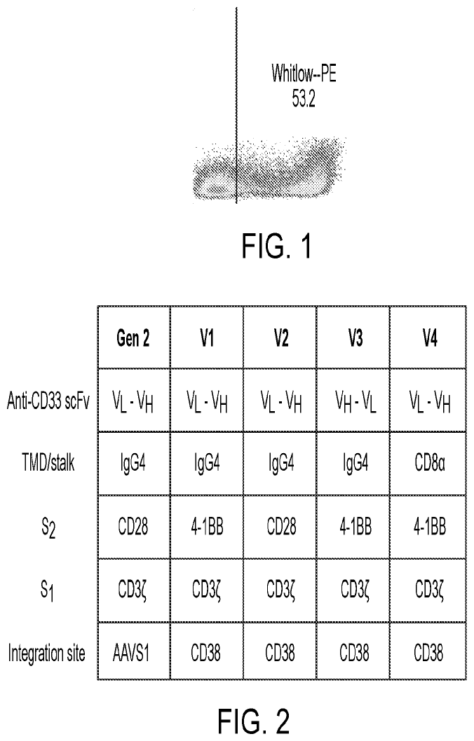

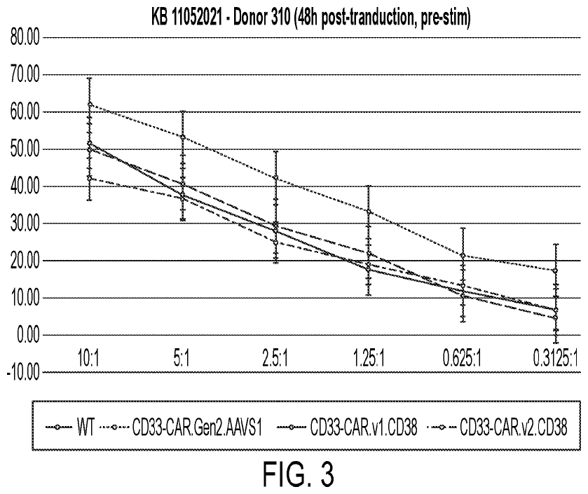

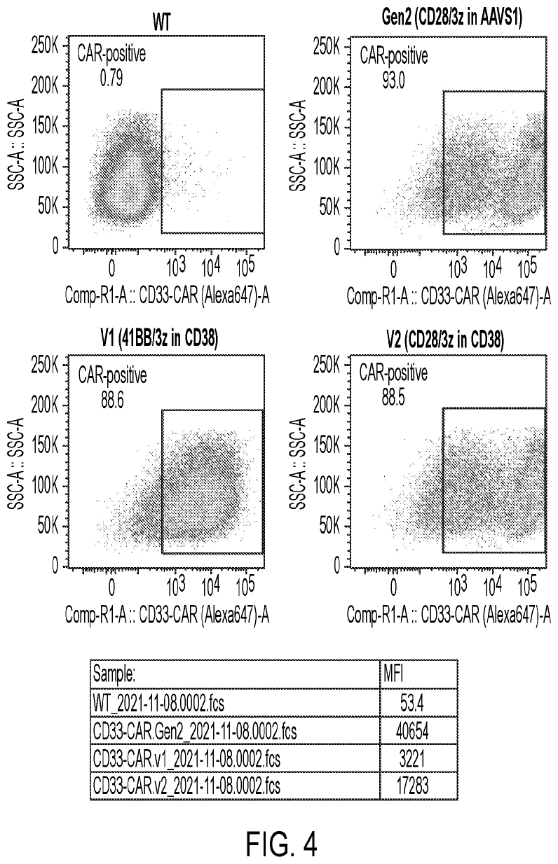

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

As used in the specification and claims, the singular form “a,” “an,” and “the” include plural references unless the context clearly dictates otherwise. For example, the term “a particle” includes a plurality of particles, including mixtures thereof.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

“Administration” to a subject includes any route of introducing or delivering to a subject an agent. Administration can be carried out by any suitable route, including oral, intravenous, intraperitoneal, intranasal, by inhalation, intravitreal, intraocular, and the like. Administration includes self-administration and the administration by another.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies, VHH single domain antibody and humanized antibodies (Harlow et al, 1999, In: Using Antibodies: A Laboratory Manual, Cold Spring Harbor Laboratory Press, NY; Harlow et al., 1989, in: Antibodies: A Laboratory Manual, Cold Spring Harbor, New York; Houston et al., 1988, Proc. Natl. Acad. Sci. USA 85:5879-5883; Bird et al., 1988, Science 242:423-426).

An antibody “heavy chain”, as used herein, refers to the larger of the two types of polypeptide chains present in all antibody molecules in their naturally occurring conformations.

An antibody “light chain”, as used herein, refers to the smaller of the two types of polypeptide chains present in all antibody molecules in their naturally occurring conformations, K and A light chains refer to the two major antibody light chain isotypes.

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, VHH, single domain antibody, and the like, including hybrid fragments. Thus, fragments of the antibodies that retain the ability to bind their specific antigens are provided. For example, fragments of antibodies which maintain prostate-specific membrane antigen (PSMA) binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “antigen” or “Ag” as used herein is defined as a molecule that provokes an immune response. This immune response may involve either antibody production, or the activation of specific immunologically-competent cells (e.g., T cells or NK cells), or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. In one example, the antigen described herein is a PSMA or a functional fragment thereof.

The term “anti-cancer effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-cancer effect” can also be manifested by the ability of the peptides, polynucleotides, cells and antibodies of the invention in prevention of the occurrence of tumor in the first place.

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is prostate cancer.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell (e.g., a T cell or an NK cell). In some embodiments, CARs comprise an intracellular domain, a transmembrane domain, and an extracellular domain comprising a tumor associated antigen binding region.

The term “CDR” as used herein refers to the “complementarity determining regions” of the antibody which consist of the antigen binding loops as defined by Kabat E. A. et al., (1991) Sequences of proteins of immunological interest. NIH Publication 91-3242.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The phrases “concurrent administration”, “administration in combination”, “simultaneous administration” or “administered simultaneously” as used herein, means that the compounds are administered at the same point in time or immediately following one another.

“Costimulatory ligand,” as the term is used herein, includes a molecule on an antigen presenting cell (e.g., dendritic cell, B cell, and the like) that specifically binds a cognate costimulatory molecule on a T cell or an NK cell, thereby providing a signal which, in addition to the primary signal, mediates a T cell or an NK cell response, including, but not limited to, proliferation, activation, differentiation, and the like. The primary signal can be provided by, for instance, binding of a T cell receptor (TCR)/CD3 complex with an MHC molecule loaded with peptide or binding of an NK cell receptor with a ligand thereof. A costimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6. ILT3, ILT4, HVEM, an agonist or antibody that binds Toll ligand receptor and a ligand that specifically binds with B7-H3.

A “costimulatory molecule” refers to the cognate binding partner on a T cell or an NK cell that specifically binds with a costimulatory ligand, thereby mediating a costimulatory response by the T cell or the NK cell, respectively.

A “costimulatory signal”, as used herein, refers to a signal, which in combination with a primary signal, such as T cell receptor (TCR)/CD3 ligation or an NK cell receptor ligation, leads to the immune cell proliferation and/or upregulation or downregulation of key molecules. The “costimulatory signaling domain” refers to a portion of the CAR comprising the intracellular domain of a costimulatory molecule.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Pharmaceutically acceptable” can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

As used herein, the terms “prevent”, “preventing” and “prevention” refer to the prevention of the recurrence or the onset of one or more symptoms of a disorder or disease, especially in individuals which have been analyzed to be susceptible or likely to develop the disease.

The term “promoter” as used herein is defined as a DNA sequence recognized by the synthetic machinery of the cell, or introduced synthetic machinery, required to initiate the specific transcription of a polynucleotide sequence.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “specificity” refers to the number of different types of antigens or antigenic determinants to which a particular antigen-binding molecule (such as the CAR, single-chain variable fragment, or PSMA binding domain of the invention) can bind. By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific, in another example, an antibody that specifically binds to an antigen may also bind to different allelic forms of the antigen, However, such cross reactivity does not itself alter the classification of an antibody as specific.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex.

The term “subject” refers to a human in need of treatment for any purpose, and more preferably a human in need of treatment to treat prostate cancer. The term “subject” can also refer to non-human animals, such as non-human primates.

As used herein, an “scFv” is a single chain variable fragment of immunoglobulin or antigen receptor. A variable domain of each of the heavy (VH) and light (VL) chain connected in some embodiments by a multi-residue peptide linker.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder (e.g., prostate cancer). The terms “treating” and “treatment” can also refer to reduction in severity and/or frequency of symptoms, elimination of symptoms and/or underlying cause, and improvement or remediation of damage.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of prostate cancer. In some embodiments, a desired therapeutic result is the control of prostate cancer, a decrease of tumor volume, or a symptom of prostate cancer. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, lentiviral vectors, adenoviral vectors, adeno-associated virus vectors, retroviral vectors, and the like.

### Compositions, Nucleic Acids, and Expression Vectors

The present disclosure provides compositions comprising the heavy chain of the variable region of an antibody, an optional linker, and the light chain of the variable region of the antibody.

The present VH-VL “reverse orientation” single-chain variable fragments are structurally distinctive and have unexpected and surprising activity compared to the (from N-terminus to C-terminus) “natural orientation” VL-VH single-chain variable fragments.

Further embodiments of the disclosure provide related nucleic acids, recombinant expression vectors, host cells, populations of cells, and pharmaceutical compositions relating to the CAR constructs of the invention.

The present disclosure provides nucleic acids, expression vectors, cells, and compositions thereof comprising an scFv for treating pathologies related to or caused by CD33.

In some aspects, disclosed herein is a composition (such as, for example a chimeric antigen receptor (CAR) composition of any disclosed aspect), comprising a single-chain variable fragment (scFv) comprising a heavy chain variable region (VH), a light chain variable region (VL), and an acceptable carrier (including, but not limited to any excipient, diluent, filler, salt, buffer, stabilizer, solubilizer, lipid, stabilizer, or other material well known in the art for use in pharmaceutical formulations), wherein the VH comprises one, two, or three heavy chain complementarity determining regions (CDRHs), and the VL comprises one, two, or three light chain complementarity determining regions (CDRLs), and wherein the CDRHs or the CDRLs comprise 70%, 75%, 80%, 85%, 90%, 95%, 99%, or 100% sequence identity to SEQ ID NO: 16, SEQ ID NO: 17, SEQ ID NO: 18, SEQ ID NO: 19, SEQ ID NO: 20, or SEQ ID NO: 21.

In some embodiments, the scFv of any preceding aspect comprises 70%, 75%, 80%, 85%, 90%, 95%, 99%, or 100% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, or any fragments thereof. In some embodiments, the scFv of any preceding aspect comprises one or more nucleic acid sequences selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, or any fragments thereof.

In some embodiments, the VH comprises 70%, 75%, 80%, 85%, 90%, 95%, 99%, or 100% sequence identity to SEQ ID NO: 1, SEQ ID NO: 2, SEQ ID NO: 3, SEQ ID NO: 4, SEQ ID NO: 5 or SEQ ID NO: 6, and the VL comprises 70%, 75%, 80%, 85%, 90%, 95%, 99%, or 100% sequence identity to SEQ ID NO: 7, SEQ ID NO: 8, SEQ ID NO: 9, SEQ ID NO: 10, SEQ ID NO: 11 or SEQ ID NO: 12.

In some embodiments, the composition further comprises a linker attached to the VH and the VL, wherein the linker comprises 80%, 85%, 90%, 95%, 99%, or 100% sequence identity to SEQ ID NO: 13. In some embodiments, the composition further comprises 80%, 85%, 90%, 95%, 99%, or 100% sequence identity to SEQ ID NO: 23, SEQ ID NO: 24, SEQ ID NO: 25, SEQ ID NO: 26, SEQ ID NO: 27, or SEQ ID NO: 28.

In some embodiments, the composition of any preceding aspect comprises a chimeric antigen receptor (CAR).

In some aspects, disclosed herein is a nucleic acid encoding the scFv of any preceding aspect.

In some aspects, disclosed herein is an expression vector comprising a nucleic acid encoding the scFv of any preceding aspect. In some embodiments, the expression vector comprises a plasmid or a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a chimeric polypeptide is a naked DNA or is comprised in a nanoparticle (e.g., liposomal vesicle, porous silicon nanoparticle, gold-DNA conjugate particle, polyethyleneimine polymer particle, cationic peptides, etc.).

### Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### Adeno-Asscociated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

### Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson, Curr Opin Mol Ther 5:633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

In some aspects, disclosed herein is a cell comprising the composition or the expression vector of any preceding aspect. In some embodiments, the cell of any preceding aspect includes, but is not limited to a T-cell, a natural killer (NK) cell, a genetically-modified T-cell, or a genetically-modified NK cell.

### Methods

Additional embodiments of the disclosure provides methods of detecting the presence of cancer in a mammal and methods of ameliorating, treating, or preventing cancer in a mammal.

In some aspects, disclosed herein is a method of treating, preventing, ameliorating, reducing, and/or decreasing a CD33-associated pathology (including, but not limited to a hematologic cancer and a neurodegenerative disorder) in a subject in need thereof, the method comprising administering to the subject a therapeutically effective amount of a composition (such as, for example a chimeric antigen receptor (CAR) composition of any preceding aspect) comprising a single-chain variable fragment (scFv), wherein the scFv comprises a heavy chain variable region (VH), a light chain variable region (VL), and an acceptable carrier, wherein the VH comprises one, two, or three heavy chain complementarity determining regions (CDRHs), and the VL comprises one, two, or three light chain complementarity determining regions (CDRLs), and wherein the CDRHs or the CDRLs comprise at least 80% sequence identity to SEQ ID NO: 16, SEQ ID NO: 17, SEQ ID NO: 18, SEQ ID NO: 19, SEQ ID NO: 20, or SEQ ID NO: 21.

In some embodiments, the method of treating, preventing, ameliorating, reducing, and/or decreasing comprises the scFv comprising 70%, 75%, 80%, 85%, 90%, 95%, 99%, or 100% sequence identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, or any fragments thereof. In some embodiments, the method of treating, preventing, ameliorating, reducing, and/or decreasing comprises the scFv comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, or any fragments thereof.

In some embodiments, the method of treating, preventing, ameliorating, reducing, and/or decreasing comprises the VH comprising 70%, 75%, 80%, 85%, 90%, 95%, 99%, or 100% sequence identity to SEQ ID NO: 1, SEQ ID NO: 2, SEQ ID NO: 3, SEQ ID NO: 4, SEQ ID NO: 5 or SEQ ID NO: 6, and the VL comprises 60%, 65%, 70%, 75%, 80%, 85%, 90%, 95%, 99%, or 100% sequence identity to SEQ ID NO: 7, SEQ ID NO: 8, SEQ ID NO: 9, SEQ ID NO: 10, SEQ ID NO: 11 or SEQ ID NO: 12.

In some embodiments, the method of treating, preventing, ameliorating, reducing, and/or decreasing further comprises a linker attached to the VH and the VL, wherein the linker comprises 80%, 85%, 90%, 95%, 99%, or 100% sequence identity to SEQ ID NO: 13. In some embodiments, the method of treating, preventing, ameliorating, reducing, and/or decreasing further comprises at least 80% sequence identity to SEQ ID NO: 23, SEQ ID NO: 24, SEQ ID NO: 25, SEQ ID NO: 26, SEQ ID NO: 27, or SEQ ID NO: 28.

In some embodiments, the method of treating, preventing, ameliorating, reducing, and/or decreasing comprises the composition comprising a chimeric antigen receptor (CAR).

The composition of any preceding aspect may be administered in such amounts, time, and route deemed necessary in order to achieve the desired result. The exact amount of the composition of any preceding aspect composition will vary from subject to subject, depending on the species, age, and general condition of the subject, the severity of any disclosed disease or disorder, the particular composition of any preceding aspect, its mode of administration, its mode of activity, and the like. The composition of any preceding aspect is preferably formulated in dosage unit form for ease of administration and uniformity of dosage. It will be understood, however, that the total daily usage of the composition of any preceding aspect will be decided by the attending physician within the scope of sound medical judgment. The specific therapeutically effective dose level for any particular subject will depend upon a variety of factors including the disease or disorder being treated and the severity of the symptoms from any disclosed disease or disorder; the activity of the composition of any preceding aspect employed; the specific composition of any preceding aspect employed; the age, body weight, general health, sex and diet of the patient; the time of administration, route of administration, and rate of excretion of the specific composition of any preceding aspect employed; the duration of the treatment; drugs used in combination or coincidental with the specific composition of any preceding aspect employed; and like factors well known in the medical arts.

The composition of any preceding aspect may be administered by any route. In some embodiments, the composition of any preceding aspect is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition of any preceding aspect.

The exact amount of composition of any preceding aspect required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is a composition of any preceding aspect and a pharmaceutically acceptable carrier selected from an excipient, a diluent, a salt, a buffer, a stabilizer, a lipid, an emulsion, a nanoparticle, and a cream.

In some embodiments, the composition of any preceding aspect is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times. In some embodiments, the composition of any preceding aspect is administered daily. In some embodiments, the composition of any preceding aspect is administered every day, every 2 days, every 3 days, every 4 days, every 5 days, every 6 days, every 7 days, or more. In some embodiments, the composition of any preceding aspect is administered every week, every 2 weeks, every 3 weeks, every 4 weeks, or more. In some embodiments, the composition of any preceding aspect is administered every month, every 2 months, every 3 months, every 4 months, every 5 months, every 6 months, every 7 months, every 8 months, every 9 months, every 10 months, every 11 months, every 12 months, or more. In some embodiments, the composition of any preceding aspect is administered every year, every 2 years, every 3 years, every 4 years, every 5 years, or more.

In some embodiments, the method treats a hematologic cancer (such as, for example acute myelogenous leukemia, leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above), neurodegenerative disorder (such as, for example Alzheimer's disease, ataxia, Huntington's disease, Parkinson's disease, amyotrophic lateral sclerosis (ALS), Friedreich ataxia, Lewy body disease, spinal muscular atrophy, Alpers' disease, Batten disease, Cerebro-oculo-facio-skeletal syndrome, Leigh syndrome, Prion diseases, monomelic amyotrophy, multiple system atrophy, striatonigral degeneration, motor neuron disease, multiple sclerosis (MS), Creutzfeldt-Jakob disease, Parkinsonism, spinocerebellar ataxia, dementia, and other related diseases), systemic lupus erythematosus, rheumatoid arthritis, asthma, allergy, sepsis, chronic obstructive pulmonary disease, graft-versus-host disease (GVHD), eosinophilia, or osteoporosis.

In some embodiments, the composition of any preceding aspect is further administered with an anti-cancer agent, including, but not limited to interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

In some embodiments, the composition of any preceding aspect is further administered with a chemotherapeutic agent, including, but not limited to anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, and hexamethyl melamine.

The details of one or more embodiments of any preceding aspect is set forth in the accompanying drawings and the description below. Other features, objects, and advantages of the invention will be apparent from the description and drawings, and from the claims.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Unless defined otherwise, all technical and scientific terms used herein have the same meanings as commonly understood by one of skill in the art to which the disclosed invention belongs.

Those skilled in the art will recognize or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention is not limited to the particular implementations disclosed herein, but that the invention will include all implementations falling within the scope of the appended claims.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Optimization and Validation of CAR Transduction into Human Primary NK Cells Using CRISPR and AAV

Human primary natural killer (NK) cells are being widely advanced for cancer immunotherapy. However, methods for gene editing of these cells have suffered low transduction rates, high cell death, and loss of transgene expression after expansion. Here, we developed a highly efficient method for site-specific gene insertion in NK cells using CRISPR (Cas9/RNP) and AAVs. We compared AAV vectors designed to mediate gene insertion by different DNA repair mechanisms, homology arm lengths, and virus concentrations. We then validated the method for site-directed gene insertion of CD33-specific CARs into primary human NK cells. CAR transduction was efficient, its expression remained stable after expansion, and it improved efficacy against AML targets.

Human primary natural killer (NK) cells have been tested in numerous clinical trials demonstrating a high safety profile and evidence of clinical benefit for patients with cancer, which has been most widely applied to acute myelogenous leukemia (AML). Enhanced targeting of NK cells to AML through CD33 targeting has been shown with antibody Fc modifications or fusion to alternative activation domains as bi- or tri-specific NK cell engagers. CD33 targeting by T cells has been enabled by CD33-targeting chimeric antigen receptors (CARs), but clinical application has been hindered by concerns of long-term suppression of hematopoiesis with CAR-T persistence. Gene modification of NK cells to enable stable expression of a CAR can also improve their antitumor activity. However, gene modification of human peripheral-blood-derived NK cells (PB-NK) using viral or non-viral vectors has been challenging due to robust foreign DNA- and RNA-sensing mechanisms that limit the efficiency of these gene-delivery methods. Despite some improvements made in using lentiviral and retroviral transduction of human primary NK cells, the relative expression of CAR has been low. To overcome this limitation, mRNA-based gene delivery has been tested in PB-NK cells, but this only allows for transient expression of transgenes. It was recently demonstrated highly efficient gene knockout in human primary NK cells by electroporating Cas9/ribonucleoprotein complexes (Cas9/RNP). After Cas9 introduces a double-stranded break (DSB), two independent and innate DNA-repair mechanisms may be employed to repair the break: homologous directed recombination (HDR) through homology repair (HR) or non-homologous end joining (NHEJ). In the presence of a DNA template encoding a gene of interest, the exogenous gene can be integrated into the Cas9-targeting site using either of these repair mechanisms. There are several ways to provide the DNA template, including viral and non-viral methods. In non-viral approaches, the single-stranded or double-stranded DNA template is typically electroporated along with Cas9/RNP; however, it typically has a lower efficiency compared with viral transduction. For viral gene delivery, adeno-associated viruses (AAV), including AAV6, have been used safely as delivery vectors in clinical trials for primary immune cells, including T cells.

The Cas9/RNP-AAV approach has not been described in human primary NK cells. The present disclosure optimizes this approach, wherein Cas9/RNP electroporation in primary human NK cells is followed by a DNA template encoding a transgene, with or without homology arms for Cas9 targeting site, delivered using single-stranded or self-complementary AAV6. Using this approach, a simple yet highly efficient generation of stable transgene-modified human primary NK cells was achieved, including two CAR-NK cells that showed enhanced anti-AML activity. The gene-modified NK cells generated by the AAV-Cas9/RNP platform have utility for clinical applications such as CAR expression for antigen specific cancer immunotherapy and for studying NK cell biology.

### Results

### Expansion of NK Cells Improves Conditions for Gene Insertion.

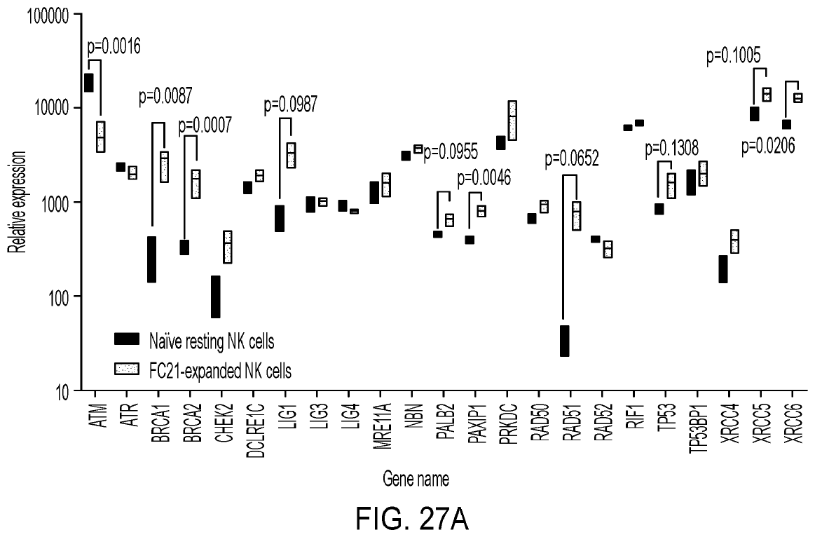

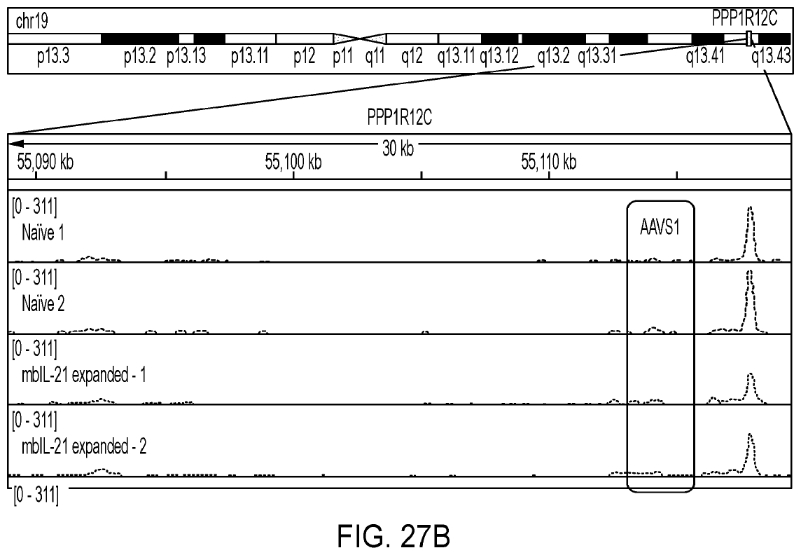

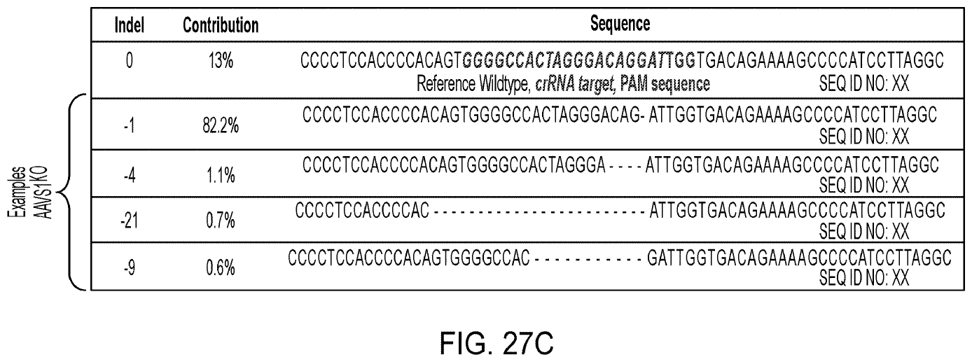

The DNA-modifying and -repairing enzymes required for NHEJ and HDR are different. NHEJ, which is essential for CRISPR-assisted insertion tagging (CRISPaint), utilizes KU80 (XRCC5), KU70 (XRCC6), DNA-PKcs (PRKDC), Artemis (DCLREIC), and LigIV (LIG4), while ATM, MRN (MRE11, NBN, and RAD50), RAD51, BRCA1, and BRCA2 are important for mediating HDR. It was previously shown that expansion of NK cells on feeder cells expressing membrane-bound interleukin-21 (IL-21; FC21) induces broad changes in gene expression. Therefore, the expression level of genes involved in HDR and NHEJ was analyzed in freshly isolated NK cells and after expansion to determine the time point at which these mechanisms would be most active. RNA sequencing (RNA-seq) showed that ATM expression decreased modestly, but all others were stable or increased, including large increases in BRCA1, BRCA2, RAD51, and LIG4 expression (FIG. 27A). This shows that conditions for both HDR- and NHEJ-directed gene insertion are active in expanded NK cells.

### Targeting a Genomic Safe Harbor for Gene Insertion.

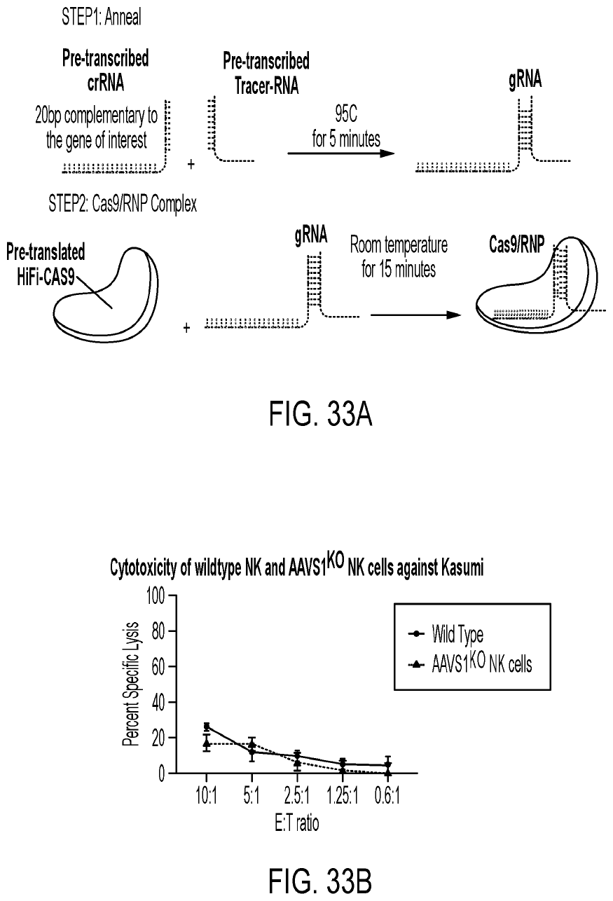

For gene insertion in NK cells, the adeno-associated virus AAV integration site 1 (AAVS1) was chosen, which is an exemplary genomic safe-harbor locus within the phosphatase 1 regulatory subunit 12C (PPP1R12C) gene. Chromatin accessibility of AAVS1 was similar in naive and day 7 expanded NK cells (n=2) as determined by assay for transposase-accessible chromatin (ATAC)-seq (FIG. 27B). Next, AAVS1 was targeted by electroporation of Cas9/RNP into day 7 expanded NK cells (FIG. 33A) using previously optimized electroporation parameters that yield high NK cell editing efficiency (>80%) and viability (>90%). No impact of AAVS1 deletion was observed on NK cell cytotoxicity against AML (FIG. 33B). After 48 h, the frequency of insertions and deletions (indels) in CRISPR-edited NK cells was determined using Inference of CRISPR Edits (ICE) using primers flanking the AAVS1 locus (Tables 1-3). The ICE results showed that up to 85% of CRISPR-modified NK cells had at least one indel at the AAVS1 Cas9-targeting site (FIG. 27C).

### Gene Insertion in Primary Human NK Cells Using Single-Stranded AAV6 and Cas9/RNP.

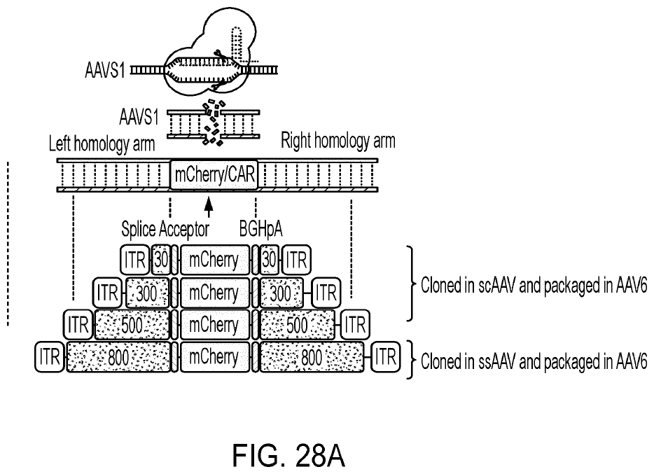

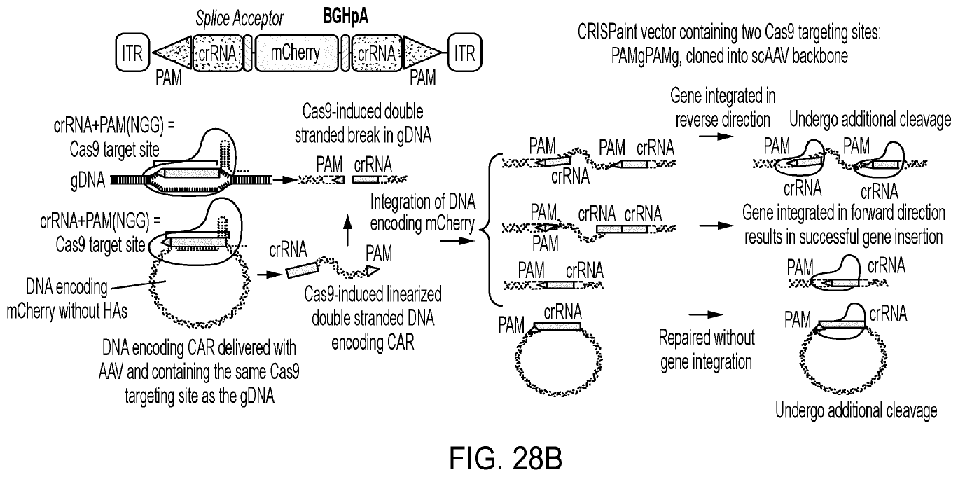

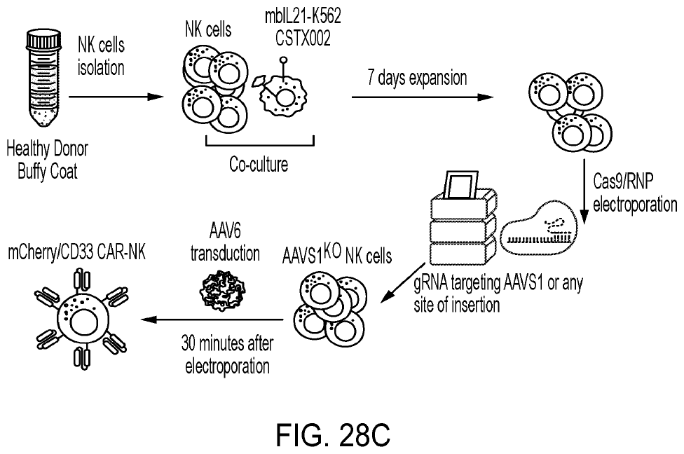

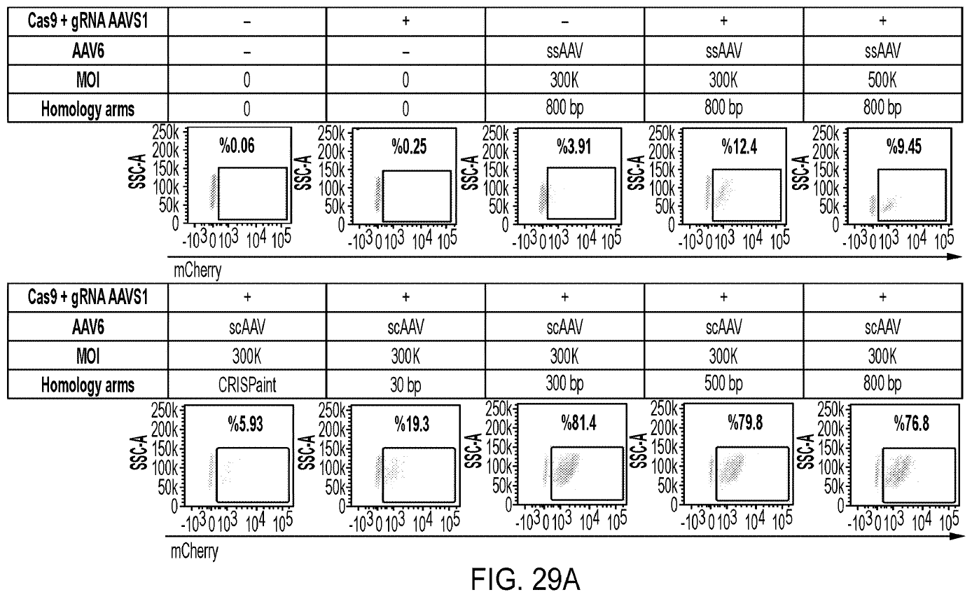

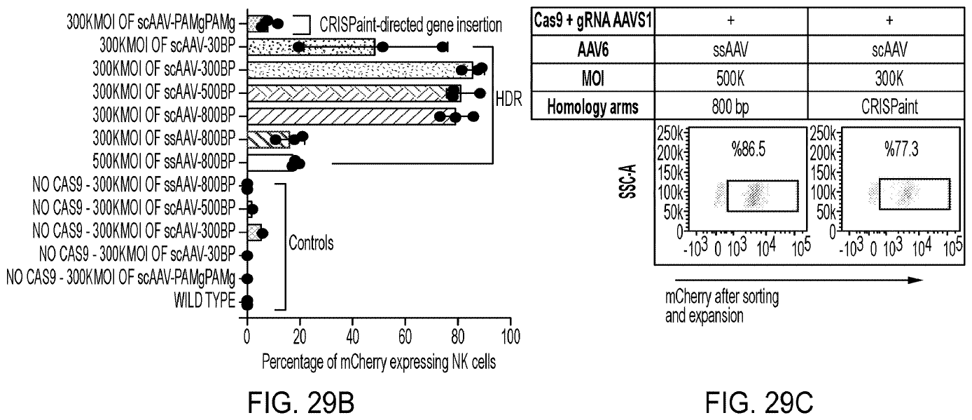

To compare the efficiency of gene insertion across DNA-repair mechanisms, a parallel series of AAV6 vectors suitable for HDR-mediated gene insertion was generated (FIG. 28A) using both single-stranded and self-complementary designs with varying homology arm lengths and for NHEJ-mediated gene insertion with CRISPaint containing PAMgPAMg sequences (FIG. 28B). To maximize HDR-mediated gene insertion, we identified homology arms (HAs) for the right and left sides of the flanking regions of the Cas9 targeting site in the AAVS1 locus, cloned these together with the mCherry gene into the backbone of a single-stranded AAV plasmid, and packaged this construct into the AAV6 viral capsid (FIG. 28A). It has been shown that the efficiency of recombination increases as the length of HAs increases. Therefore, for the single-stranded AAV (ssAAV) backbone, we used the longest possible length of the left and right HAs for mCherry (800-1,000 bp of HAs; sequences provided in Table 5). The constructs also contained a splice acceptor down-stream of the transgene to improve the transcription of the mCherry gene (FIG. 28A). Electroporation of the NK cells with Cas9/RNP targeting AAVS1 followed 30 min later by AAV trans-duction (FIG. 28C) resulted in 17% (300,000 multiplicities of infection [MOIs]) and 19% (500,000 MOIs) mCherry-positive NK cells (FIG. 29A). These cells were further expanded for 1 week using FC21, enriched the mCherry-positive cells by fluorescence-activated cell sorting (FACS), and did not see any reduction in the expression level of mCherry during an additional 30 days of expansion (FIGS. 29A, 29B, and 29C), demonstrating stable integration.

### Improved Gene Insertion Using Self-Complementary AAV6 and Cas9/RNP.

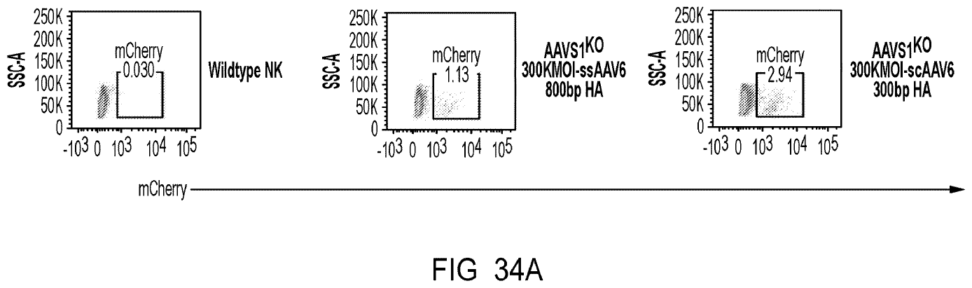

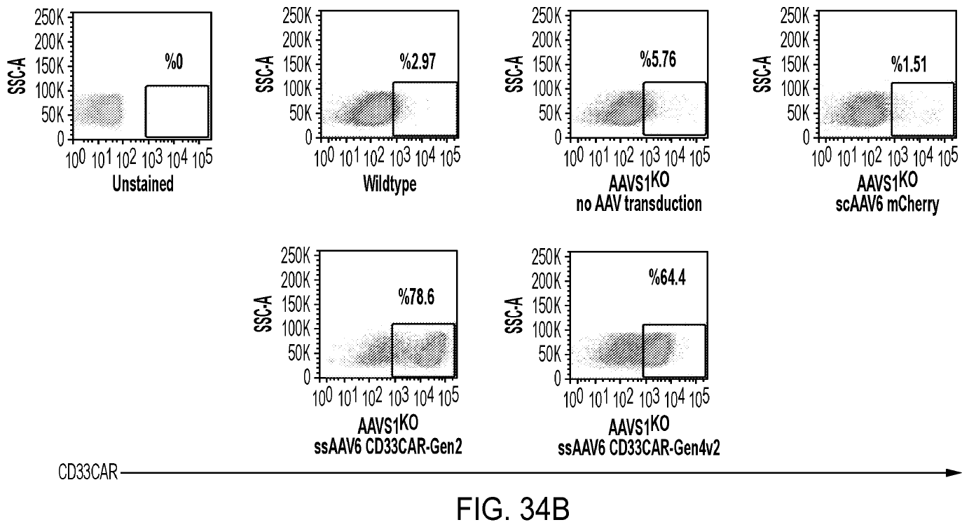

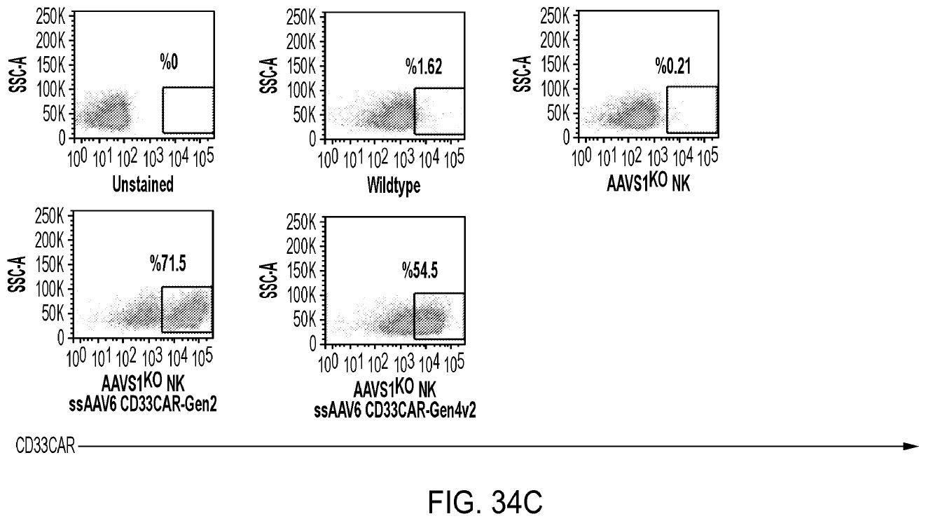

After transduction, scAAV vectors can acquire the necessary double-stranded state in a shorter time frame than ssAAV, which may impact the efficiency of gene insertion. Due to the size limitation of packaging transgenes in scAAV, HAs of varying lengths were designed to minimize the size needed for scAAV backbones. Hence, HAs of 30, 300, 500, and 1,000 bp length for the right and 30, 300, 500, and 800 bp for the left (FIG. 28A) were cloned with mCherry into the scAAV backbone and packaged into AAV6 capsid. The same steps were followed as for the ssAAV above to electroporate and transduce the day 7 expanded NK cells. scAAV with HAs R300 bp showed markedly increased efficiency of gene transfer at >80% (FIGS. 29A and 29B). Stable mCherry gene expression was observed for at least 3 weeks of additional NK cell expansion (FIG. 29C). When the same approach was used in freshly isolated NK cells, mCherry expression was significantly lower (1.13% for 800 bp ssAAV6; 2.9% for self-complementary [sc] 300 bp scAAV6; FIG. 34A).

### CRISPaint for Gene Insertion in NK Cells.

To overcome the complexity of HA optimization seen in HDR-directed gene insertion, a homology-independent gene-insertion approach called CRISPaint was tested. For the CRISPaint DNA templates, double Cas9-targeting sequences of AAVS1 (PAMgPAMg) was incorporated around the mCherry transgene but within the inverted terminal repeats (ITRs) of scAAV and packaged it into AAV6 (FIG. 28B). Two days after electroporation and transduction, flow cytometry was performed to assess mCherry expression in NK cells. The cells that were electroporated and transduced with 300,000 MOIs of scAAV6 delivering CRISPaint PAMPAMg were found to be up to 6% mCherry positive. These NK cells were further sorted, enriched, and expanded for 30 days and saw no decline in the percentage that were mCherry positive (FIGS. 29B and 29C). Although lower efficiency of gene integration was seen using CRISPaint compared with HDR-directed gene insertion, this method is still to be useful because it allows integration into a user-defined locus without designing HAs.

### Generation of Human Primary CD33-CAR NK Cells.

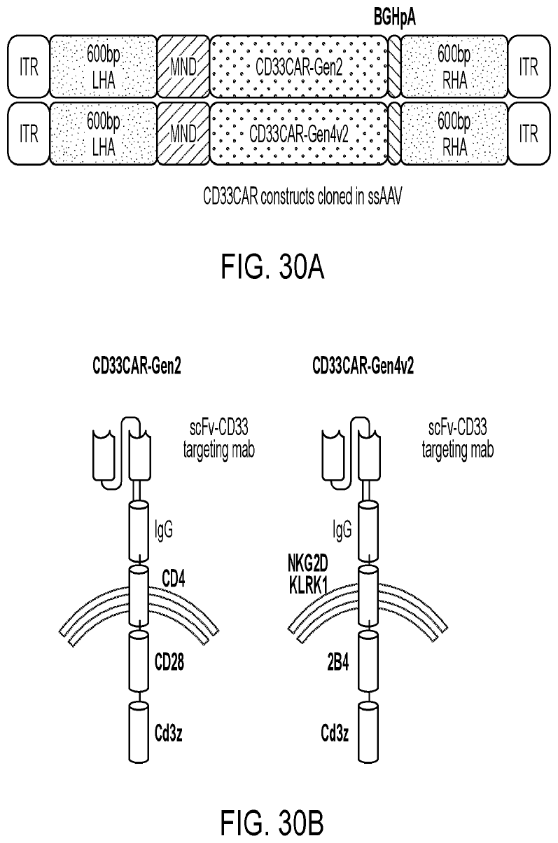

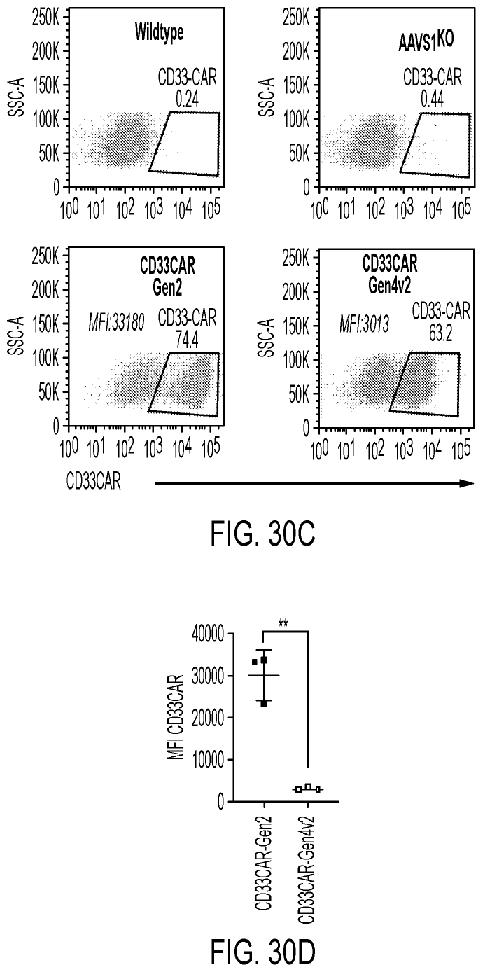

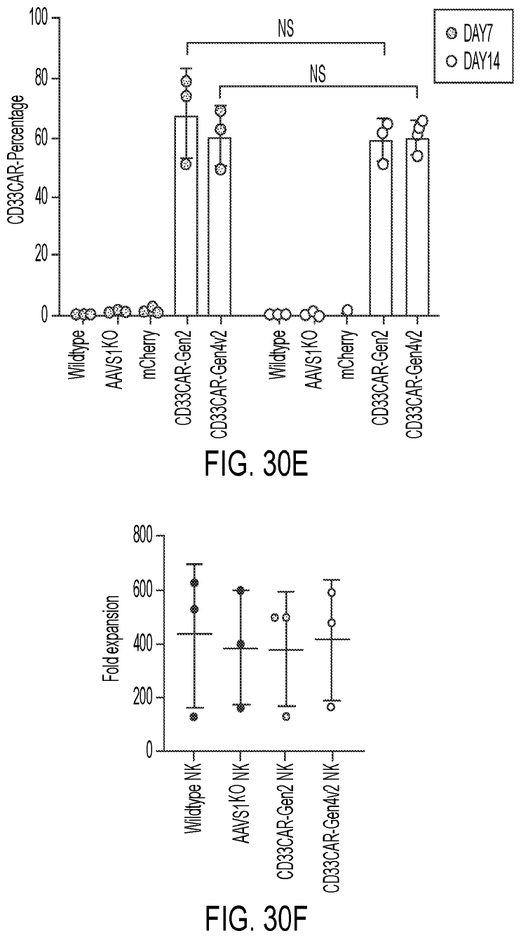

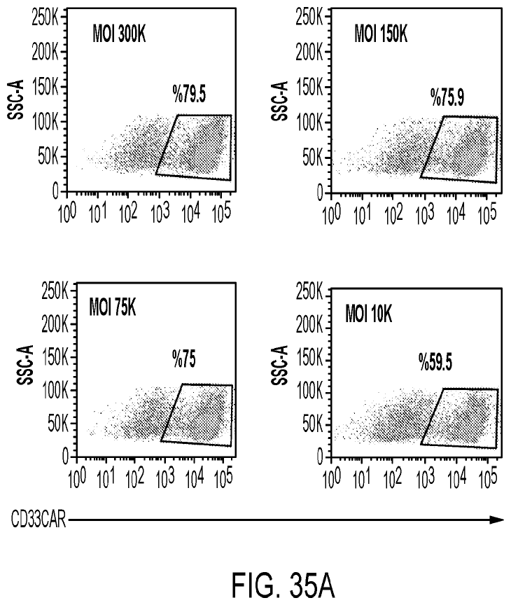

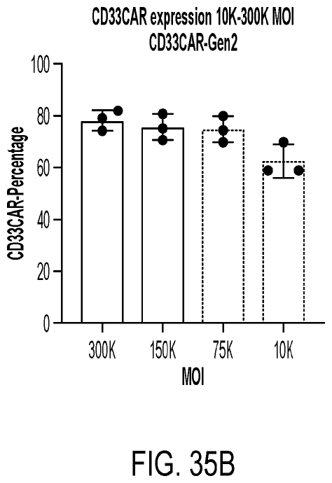

Two CAR constructs comprising the same CD33-targeting single-chain variable fragment (scFv) were tested but with a CD4 transmembrane domain and CD28/CD3z signaling domain (Gen2) or an NKG2D transmembrane domain and 2B4/CD3z signaling domain (Gen4v2) (FIGS. 30A and 30B). The CAR constructs were too large for suitable packaging into the scAAV backbone, so they were cloned into the ssAAV backbone with the largest possible HAs of 600 bp. To improve the expression of the CARs, a murine leukemia virus-derived promoter (MND) was also incorporated before the start codon of the CARs instead of the splice acceptor. As with the mCherry vectors, these were packaged into the AAV6 capsid. Seven days after electroporation and transduction, up to 78% CD33 CAR-expressing NK cells (mean 59.3% for Gen2 and 60% for Gen4v2) were detected. Of note, the CD33CAR-Gen2 resulted in a higher level of expression on NK cells compared with Gen4v2 (FIGS. 30C, 30D, and 34B). The cells were expanded for another week (day 14) and observed no significant reduction in CAR expression (FIGS. 30E and 34C) or proliferative potential (FIG. 30F), showing that neither the AAV6 transduction event nor the CAR expression impacted NK cell proliferation or survival. Efficient CAR integration (>60%) was also observed with MOIs as low as 10,000 (FIG. 35), and toxicity of the AAV6 transduction was not observed across the MOIs tested.

### Detection of the Transgene at the Targeted AAVS1 Locus and Unintended Insertion Sites.

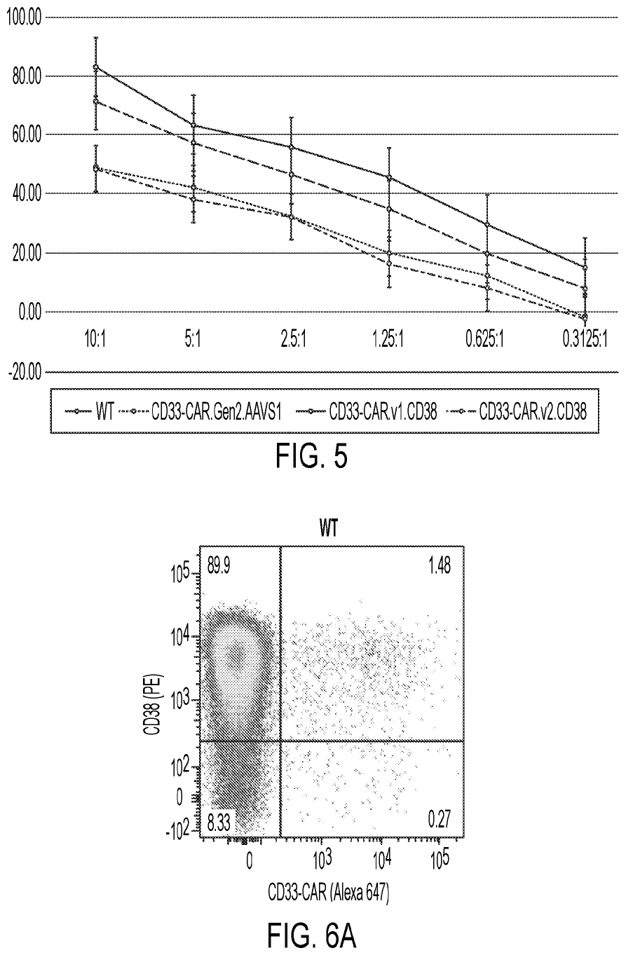

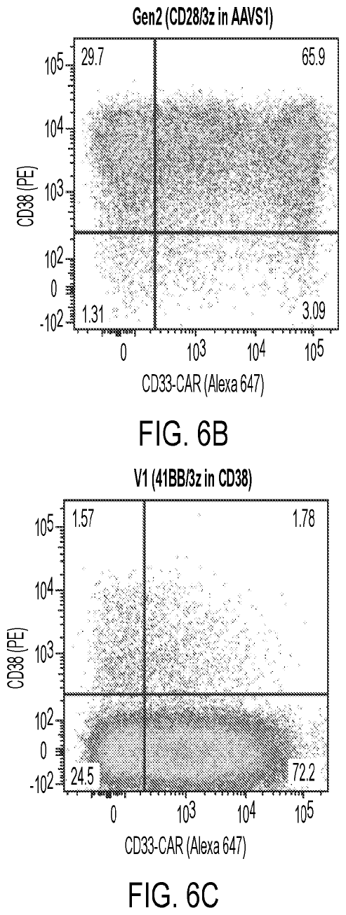

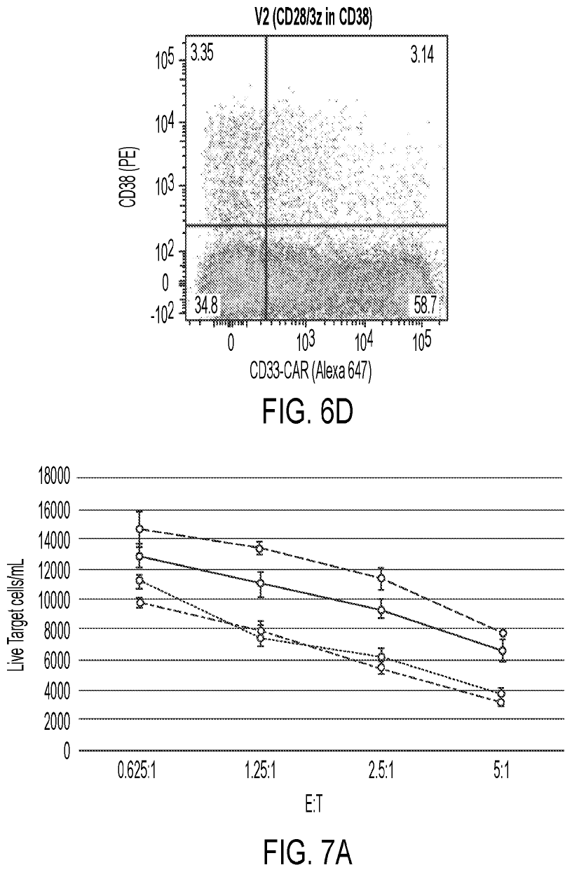

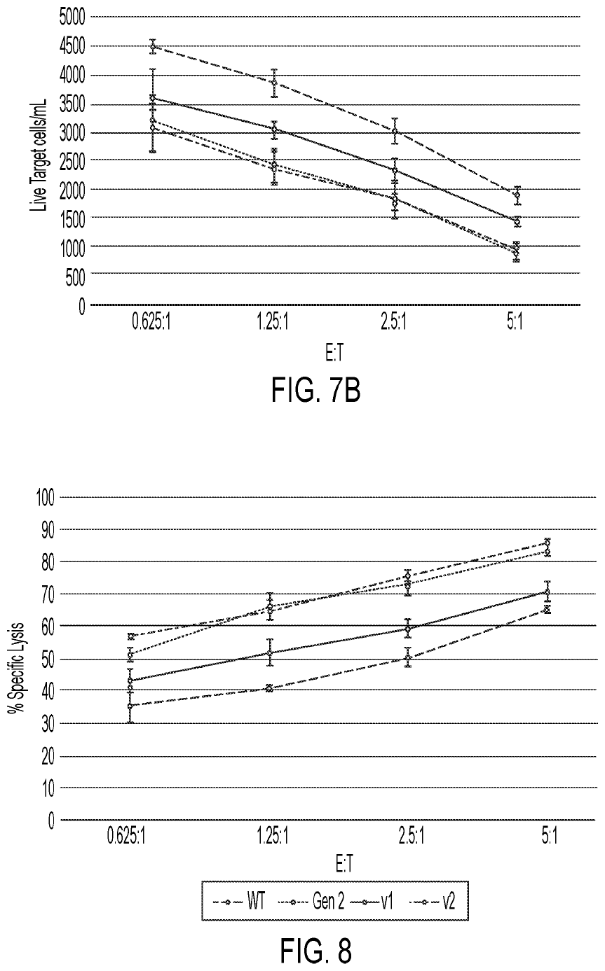

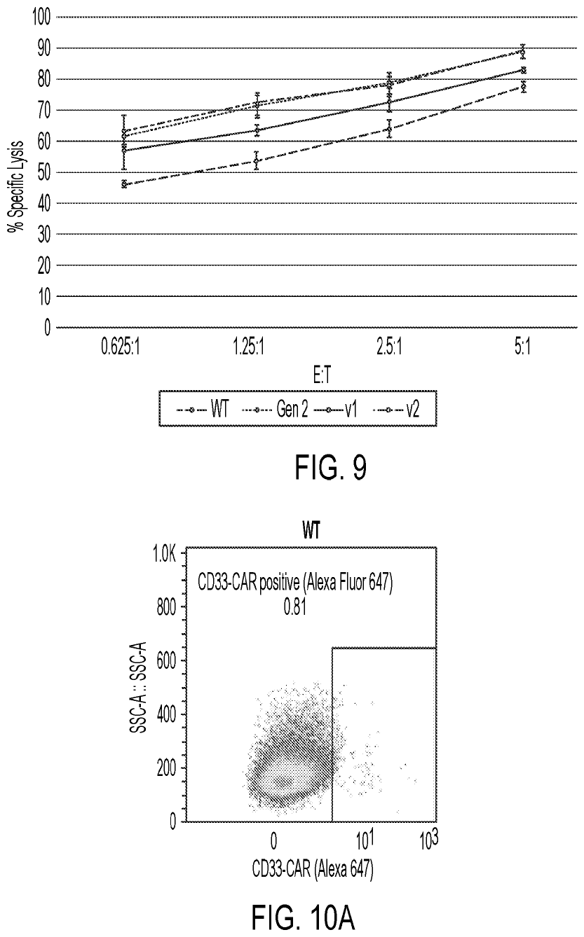

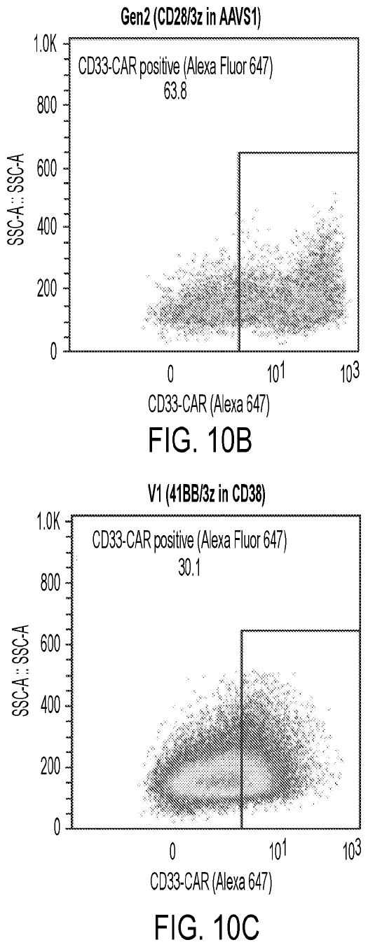

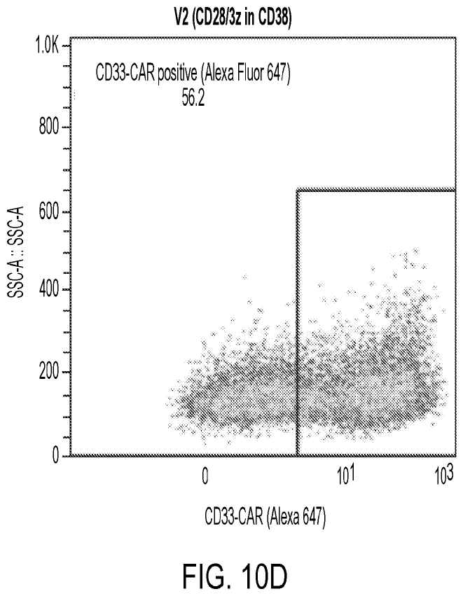

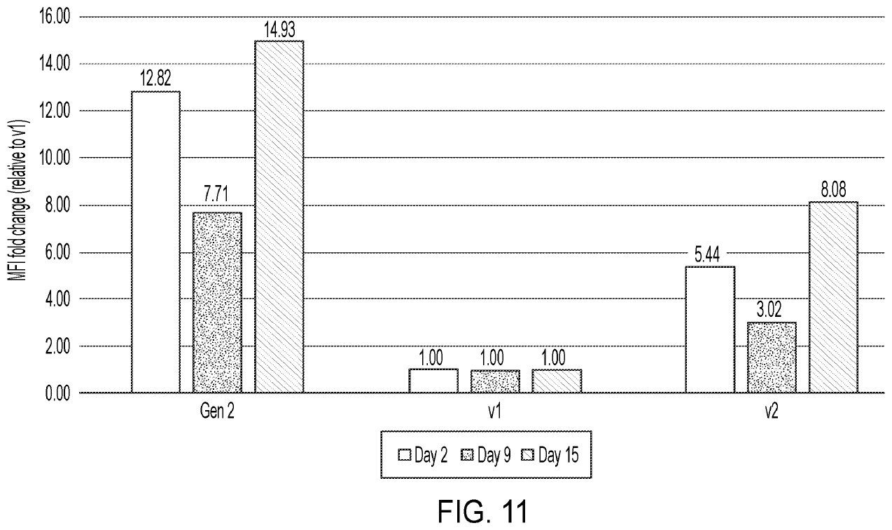

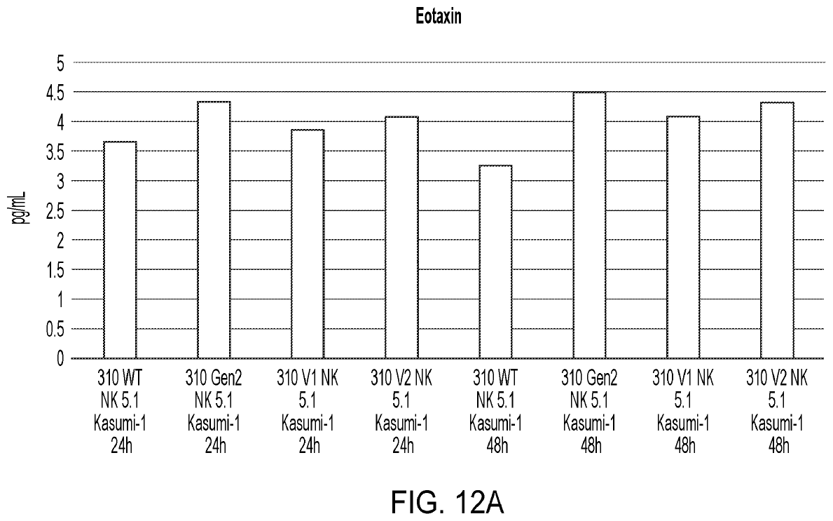

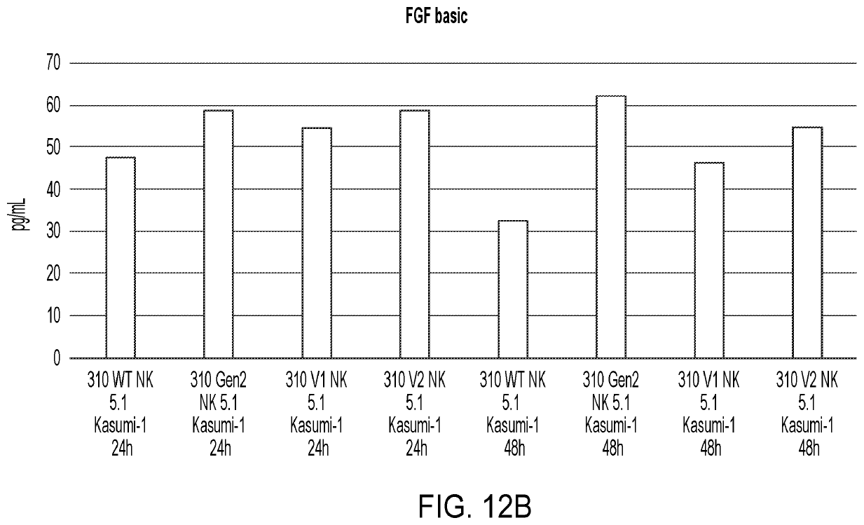

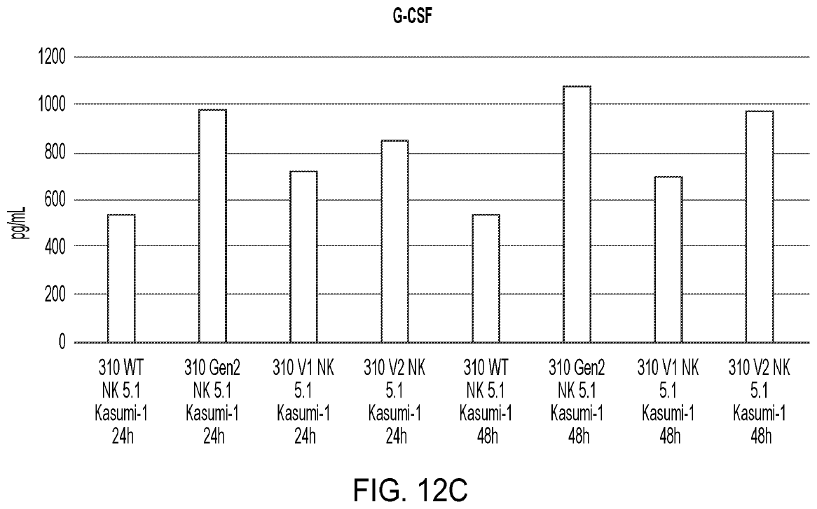

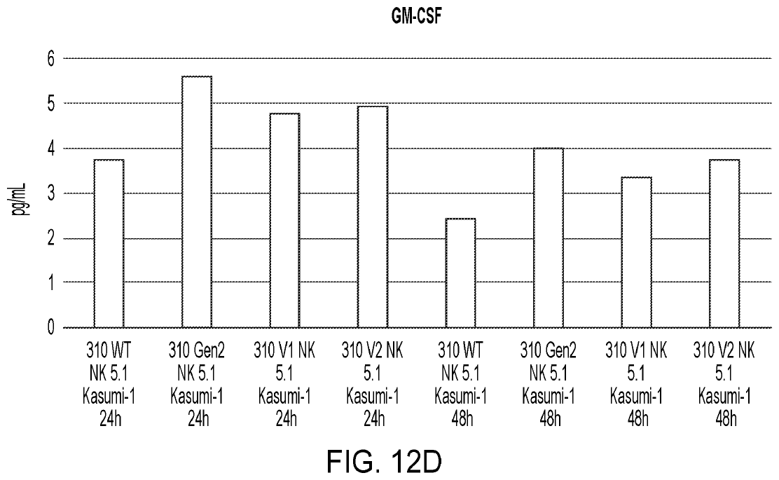

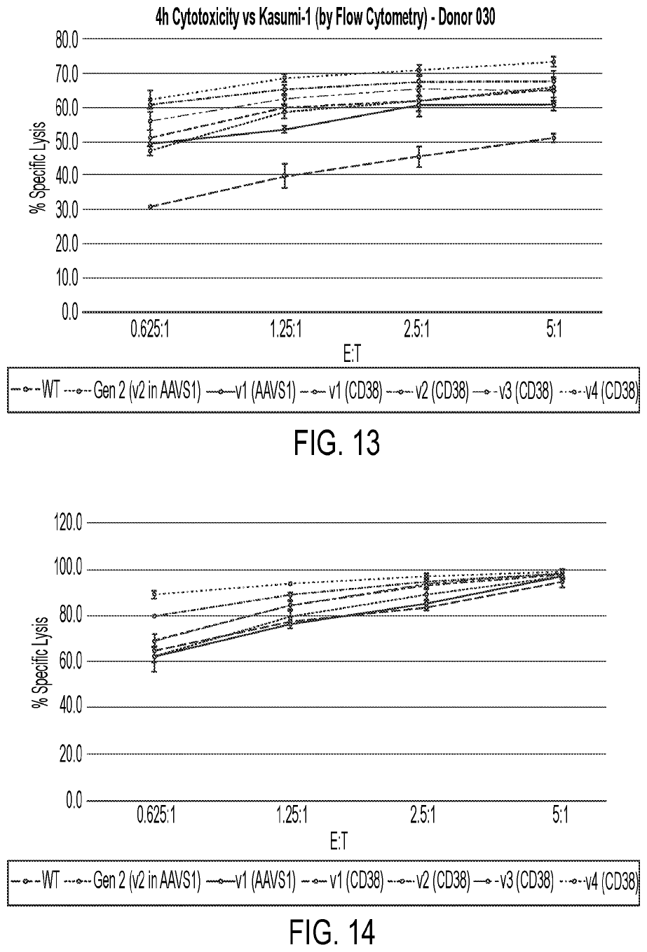

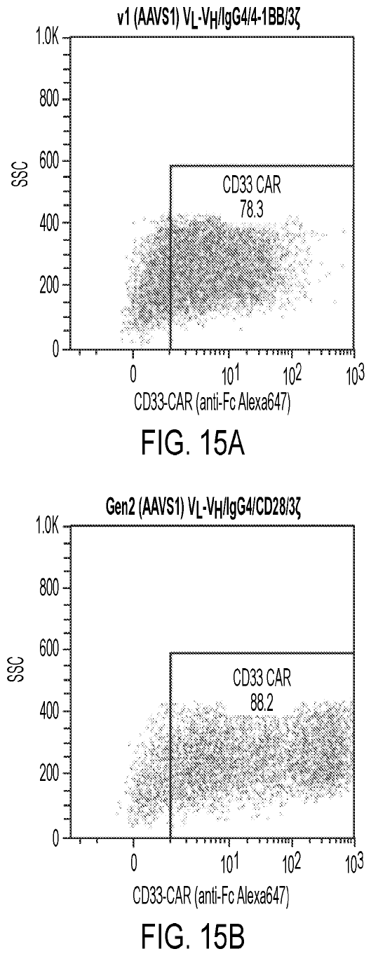

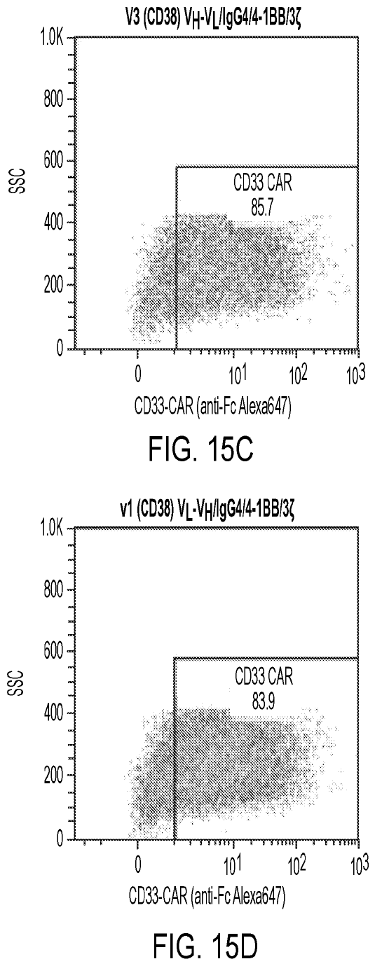

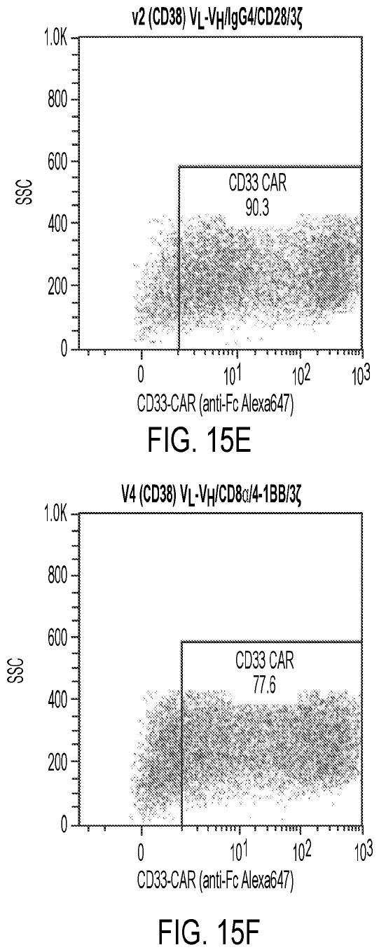

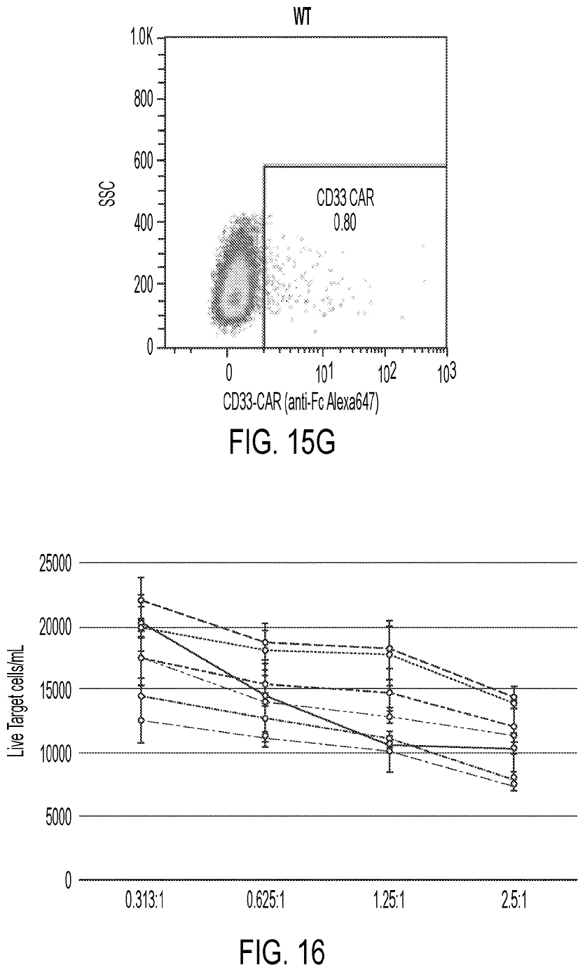

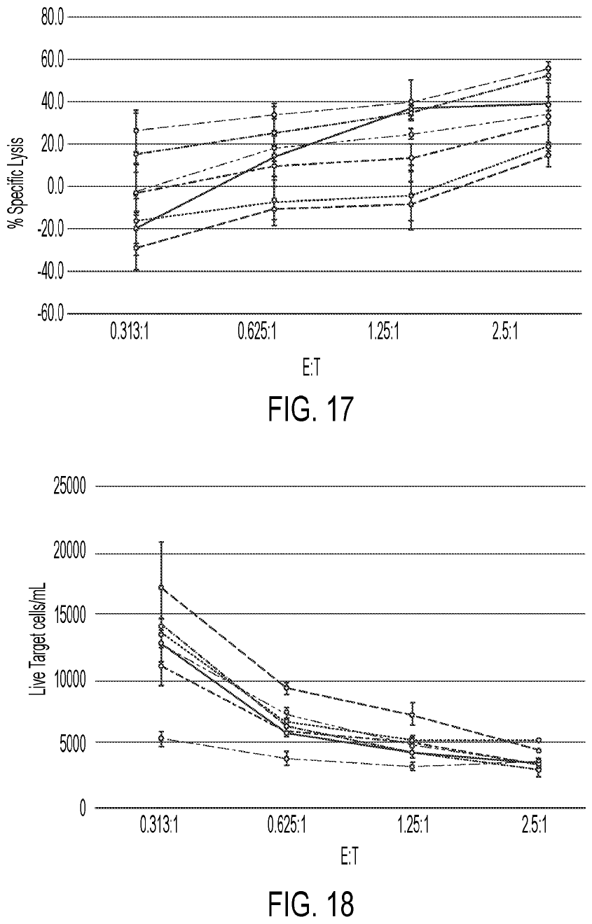

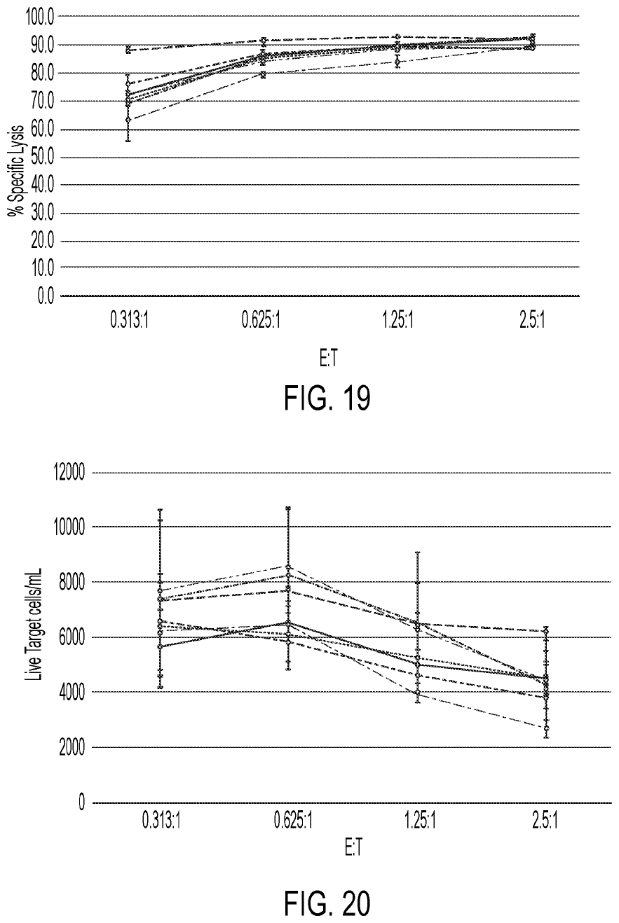

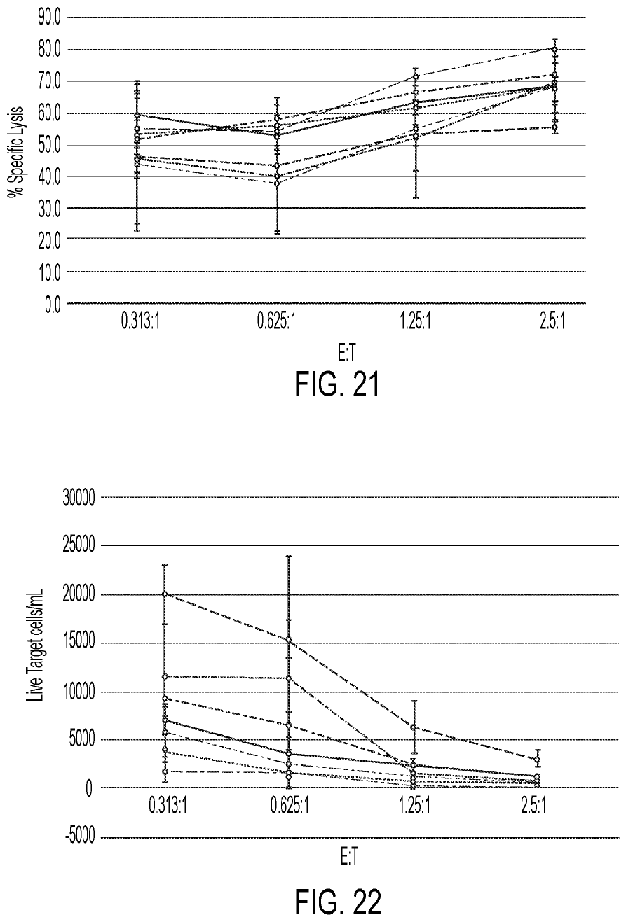

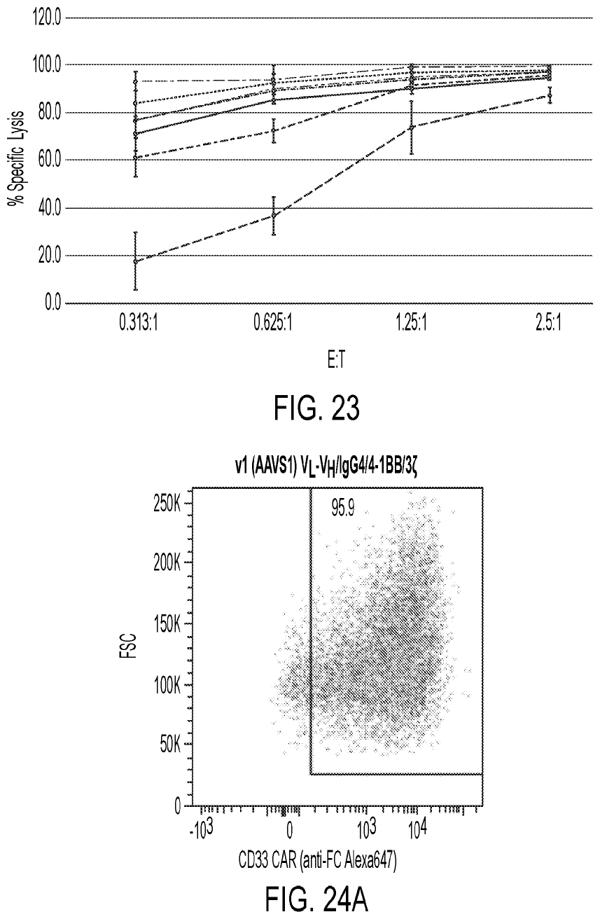

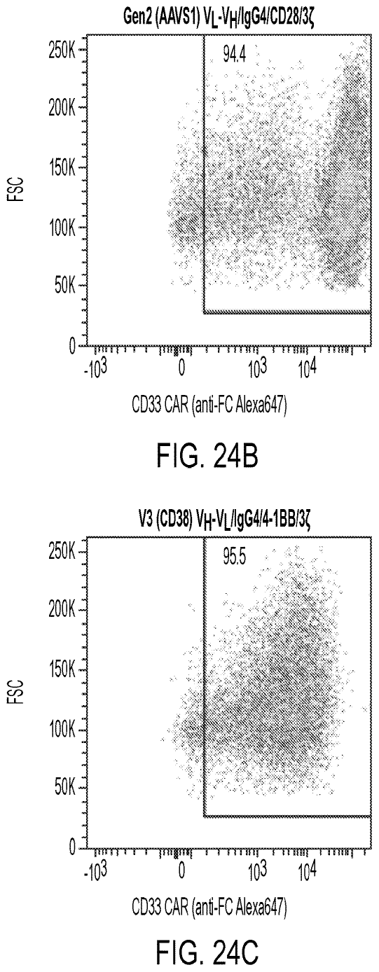

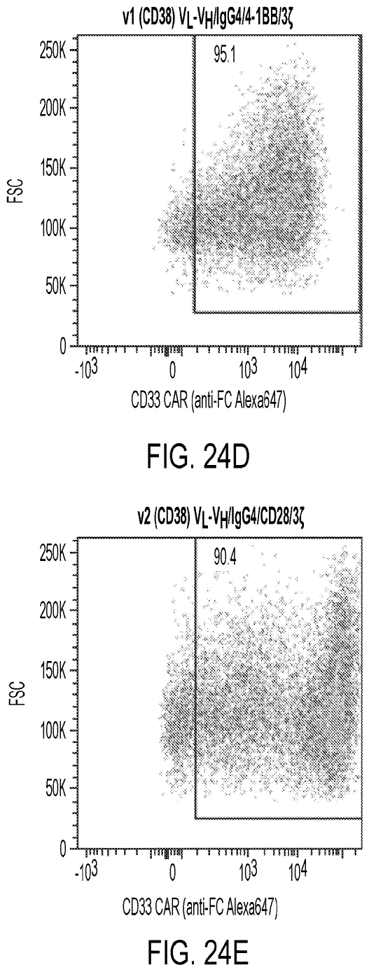

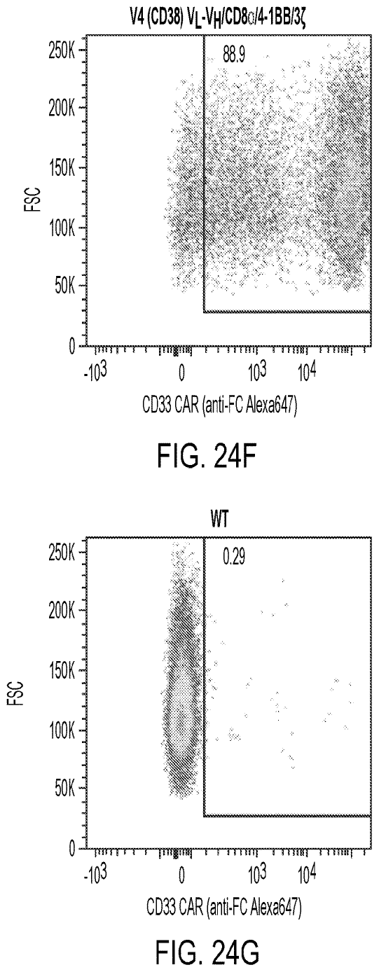

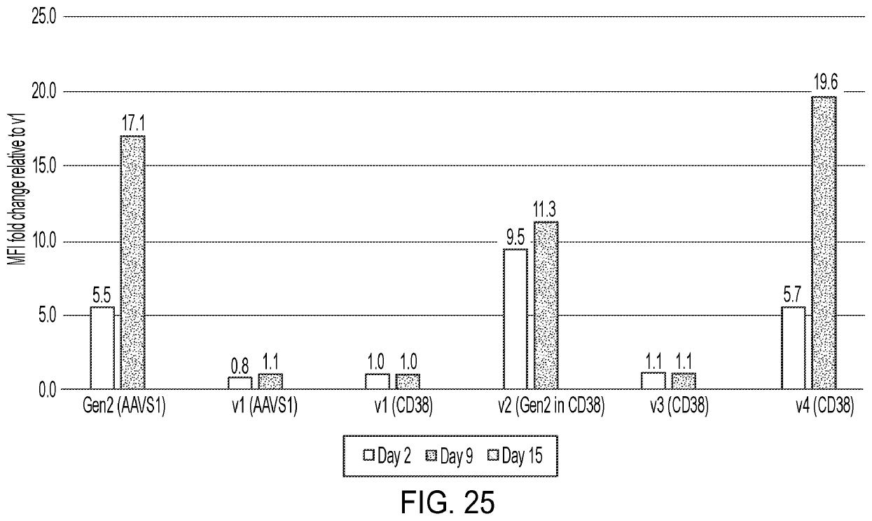

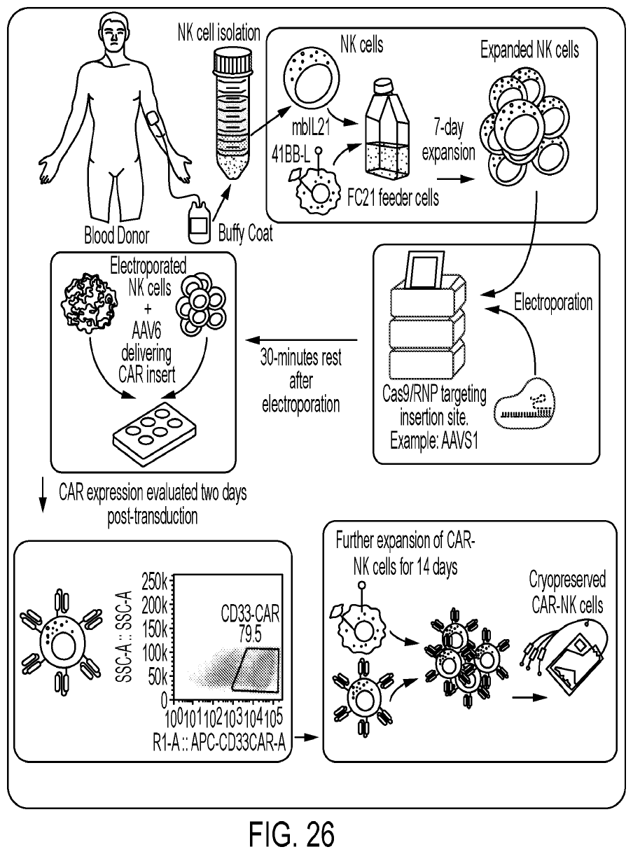

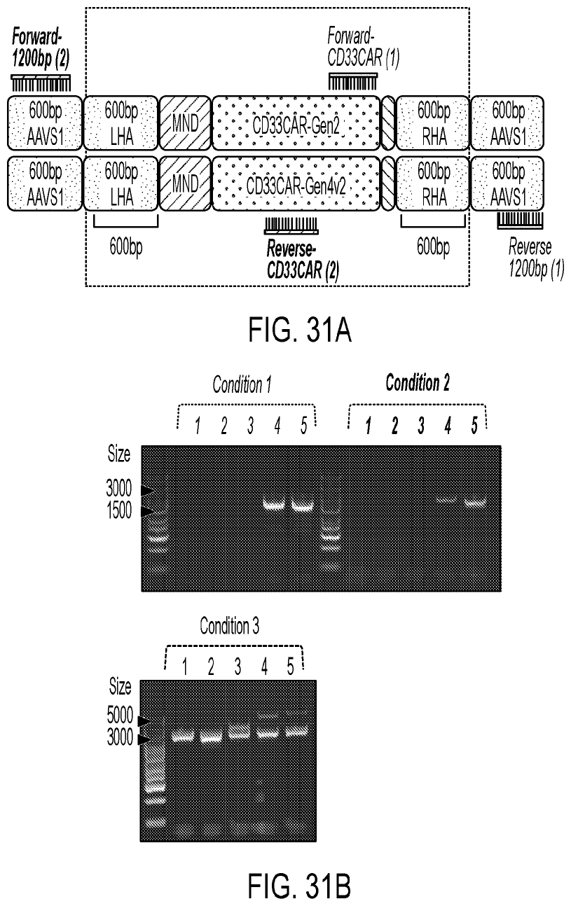

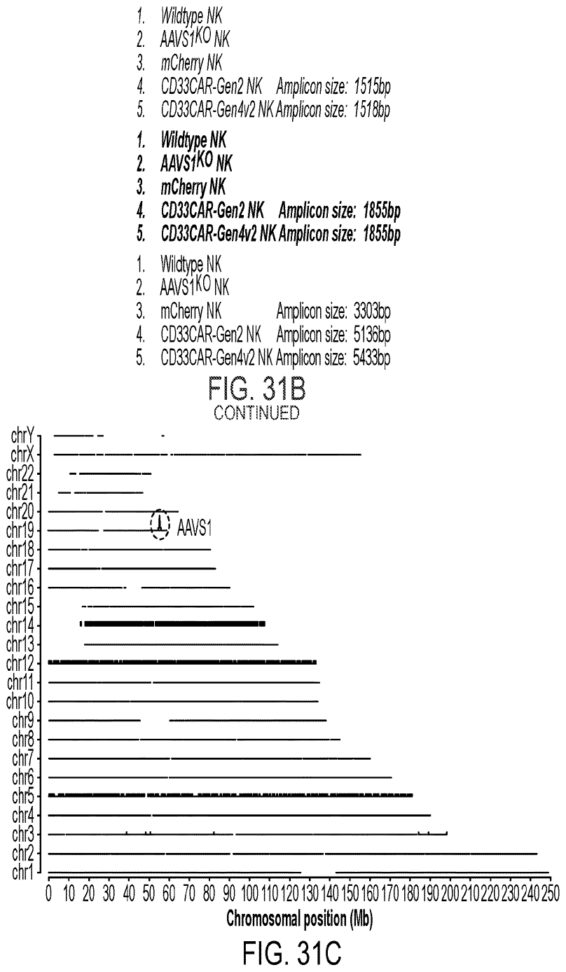

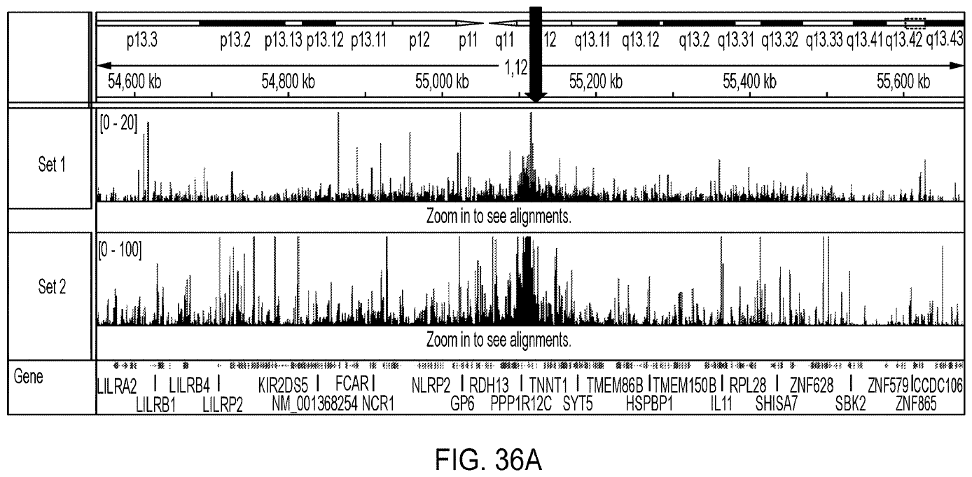

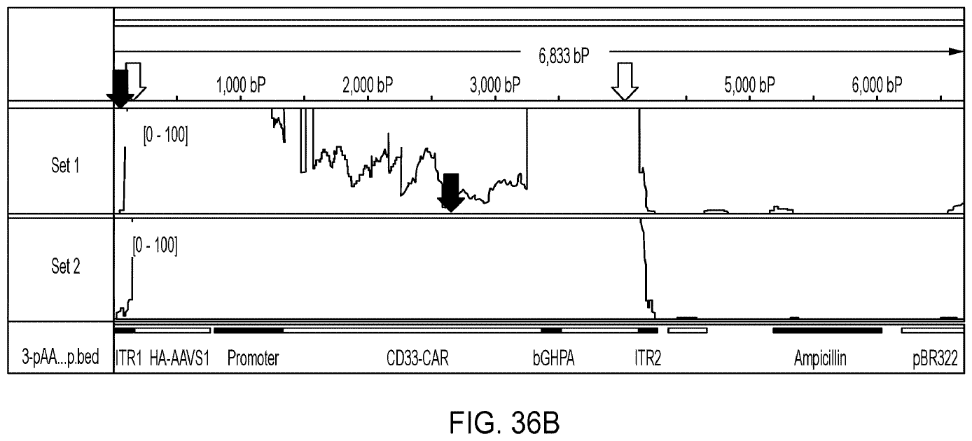

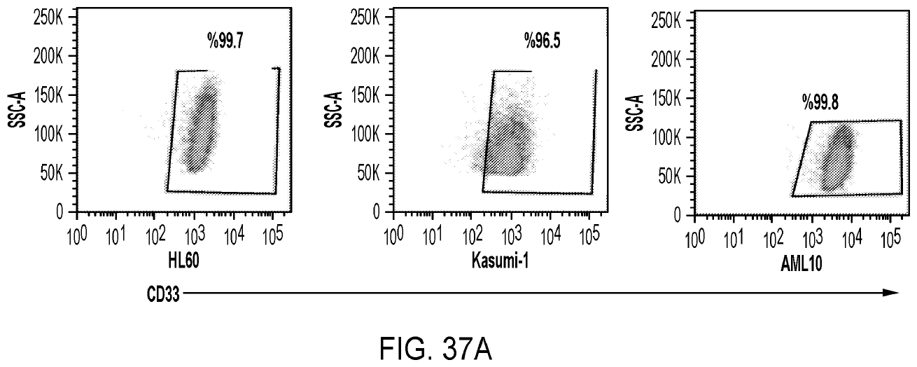

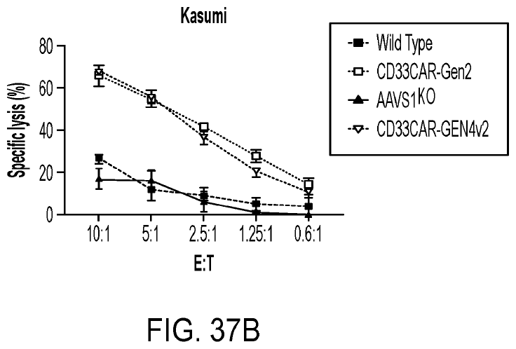

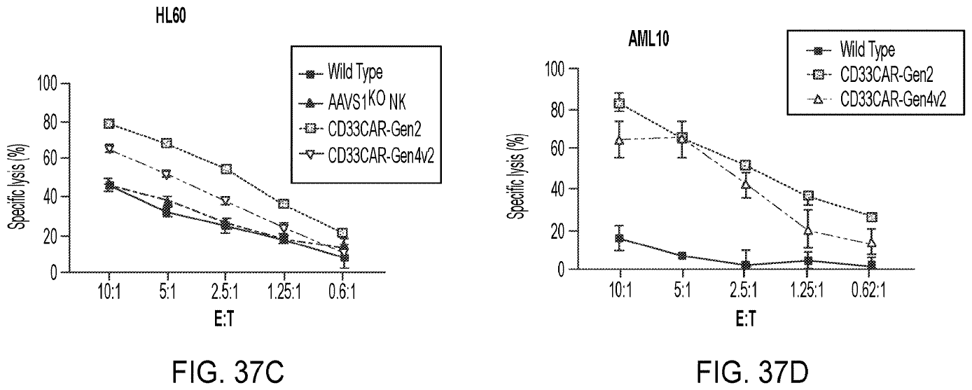

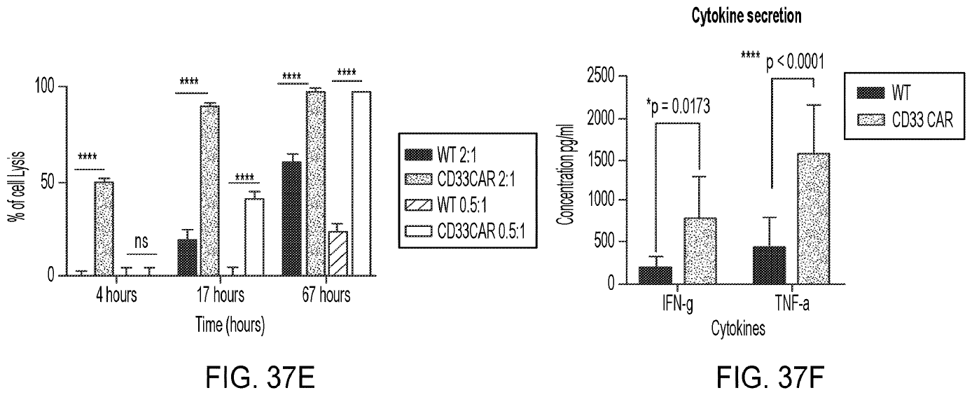

Using PCR with primers to flanking and inter-transgenic regions (FIG. 31A; Tables 1-3), the DNA integration of the transgenes (FIG. 5B) was confirmed. Additionally, targeted locus amplification (TLA) was used for whole-genome mapping of CD33CAR-Gen2 integration in CAR-expressing NK cells with a sensitivity of detecting random integrations of more than 5%. As seen in FIG. 31C, the vector has integrated as intended in human chromosome chr19: 55,115,754-55,115,767, which is in intron 1 of PPP1R12C. Other integration sites were observed between chr19: 55,115,155-55,116,371, which is also in intron 1 of PPP1R12C (FIGS. 36A and 37; Tables 1-3). Sequence variants and structural variants were identified in the covered regions (FIG. 36B). There was no indication of a dominant secondary off-target integration site. One sequence variant and four structural variants were detected (Table 4). The frequency of detection shows this variant was present within the AAV6 vector itself. Overall, the TLA demonstrated high prevalence of vector integration at the targeted location in chromosome 19, with low-level random integrations identified throughout the genome.

Human Primary CAR-NK Cells have Enhanced Antitumor Activity.

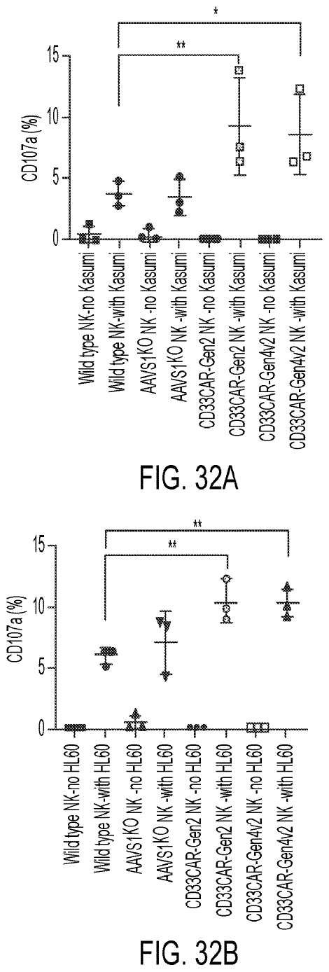

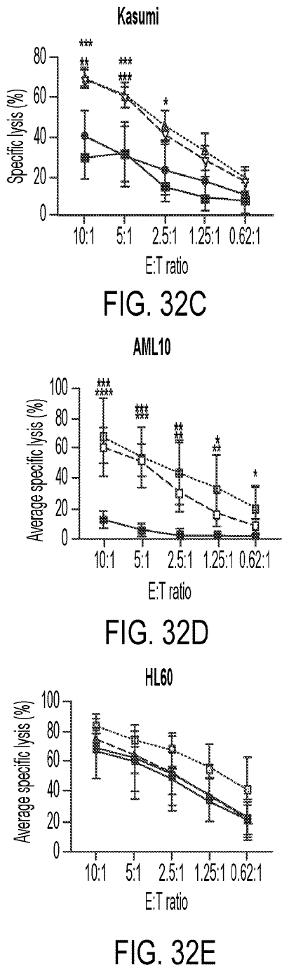

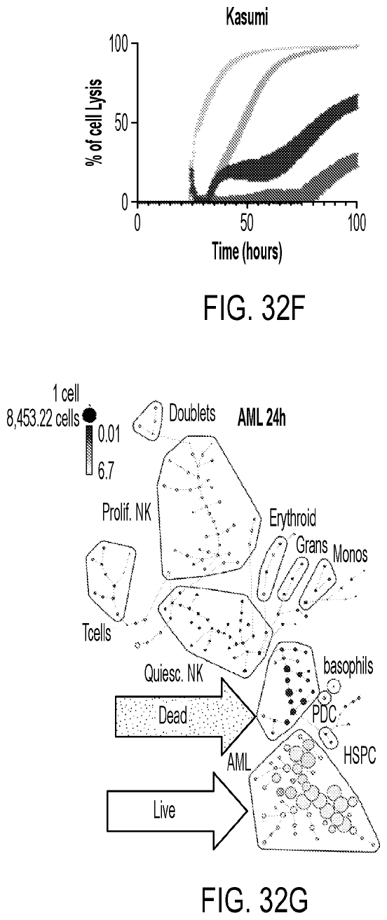

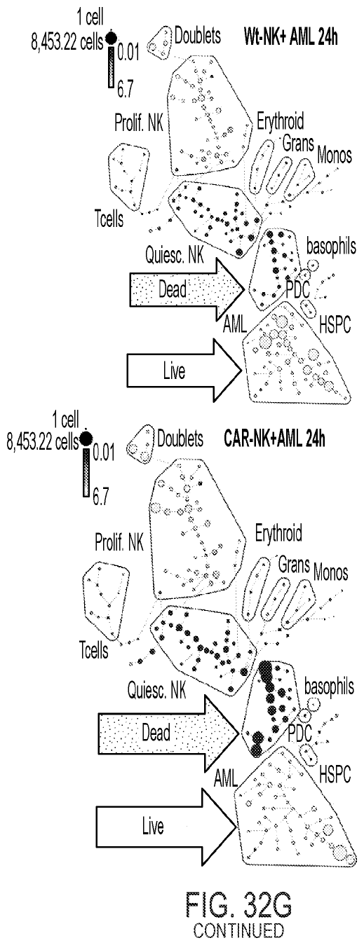

To determine whether the CD33CAR enhanced NK cell killing of AML cells, a calcein-AM-based cytotoxicity assay was performed with two CD33-expressing AML cell lines (Kasumi-1 and HL60) and one patient-derived sample (AML10) (FIG. 37A) using CD33CAR-NK cells generated from three different healthy individuals. CD33CAR-gen2 and -gen4v2 NK cells showed a significantly higher degranulation and target cell lysis when co-cultured with Kasumi-1 or HL60 cell lines compared with wild-type or AAVS1^{KO} NK cells (FIGS. 32A, 32B, 32C, and 32D). Importantly, significantly higher antitumor activity of CD33CAR NK cells against AML-10, a primary human AML sample derived from a patient with relapsed and refractory AML (FIG. 32E). Overall, CD33CAR-Gen2 NK showed better cytotoxicity compared with CD33CAR-Gen4v2 NK cells. Due to the higher antitumor activity of CD33CAR-Gen2, these CAR-NK cells were used for the rest of the functional assessments. Using real-time assessment of cytotoxicity (xCELLigence), CD33CAR-Gen2-NK cells killed the CD33-expressing AML cells more completely and with faster kinetics than the same donor wild-type (WT) NK cells (FIGS. 32F and 37B-37E). CD33CAR-Gen2 NK cells also showed significantly higher secretion of interferon gamma (IFNg) and tumor necrosis factor alpha (TNF-α) when co-cultured with Kasumi-1 compared with non-modified NK cells (FIG. 37F).

### Mass Cytometry Showed Enhanced Killing and Specificity of CD33CAR-NK Cells Against AML.

The combination of pRb and cleaved PARP was previously used to enable very accurate detection of dead or dying cells in mass cytometry with a wide variety of other extracellular and intracellular markers. Herein, the same approach was used to measure the ability of CAR-NK cells to kill AML cells. WT or CD33Gen2-CAR NK cells generated from one donor were co-cultured with primary AML and then assessed for viability across multiple cell populations. Four main populations (FIG. 32G) were identified by both manual gating and SPADE clustering: live proliferating NK cells, quiescent NK cells, live proliferating AML cells, and dying AML cells. At 3 h, control primary AML cells were ~66% viable, whereas viability decreased to 56% when co-cultured with WT-NK cells and only 21% when co-cultured with CD33CAR NK cells. At 24 h, control primary AML cells recovered to 89% viability compared with 76% when co-cultured with WT-NK cells and only 42% when co-cultured with CD33CAR NK cells. At 3 h, the surviving AML cells had a 20-fold reduction in CD33 expression when cultured with the CD33CAR NK cells (median of 104 counts down to 5 counts), while there was minimal change in CD33 expression in the WT NK cell co-culture (median of 104 counts down to 87 counts). This difference persisted at 24 h, at which time the median CD33 counts were 25 for CD33CAR NK cells, 118 for WT NK cells, and 120 in control AML without NK cells. Co-culture with AML also increased NK activation markers (CD69, CD99, CD71, NKG2D, CD16, and CD45) at 24 h compared with the NK cells cultured alone. The CD33CAR NK cells had lower levels of activation markers at baseline that increased more with co-culture. Together, these data show that CD33CAR-NK cells specifically target CD33-ex-pressing AML and are more activated by the AML targets compared with WT-NK cells (FIG. 32G).

### DISCUSSION

Gene transfer in NK cells has been challenging. Despite some progress having been made to transduce NK cells with lentiviral and retroviral vectors to generate CAR-NK cells, the efficiency of these methods remains relatively low. Herein, a highly efficient method for site-directed gene integration into human primary NK cells using a combination of electroporation of Cas9/RNP and ssAAV6 or scAAV6 gene delivery through HDR and homology-independent gene insertion (CRISPaint) is reported. The expression level of genes regulating HDR and NHEJ pathways is also shown in human NK cells generally increase during expansion, with FC21 resulting in improved conditions for site-directed gene insertion. AAVS1 is also shown to host and express exogenous genes in a very highly efficient level, as shown previously in T cells and NK cells. Furthermore, a range of HAs from 30-1,000 bp can be used for gene insertion into the AAVS1 locus in NK cells but that the shortest optimal length is at 300 bp when used in scAAV6. No difference between WT and CRISPR-modified AAVS1^{KO} NK cells was observed in degranulation or cytotoxicity against AML cell lines, which shows that genome modifications at this locus do not interfere with NK cell function (FIGS. 32C, 33B, 37B, and 37C). Although less efficient, CRISPaint gene insertion may be useful for tagging endogenous genes and therefore is useful for biologic studies in NK cells.

Transcripts that are delivered via AAV vectors can be pack-aged as a linear ssDNA with a length of approximately 4.7 kb (ssAAV) or as linear scDNA (scAAV). The benefit of the scAAV vector is that it contains a mutated ITR, which is required for replication and helps to bypass rate-limiting steps of second-strand generation compared with ssDNA vectors. Due to the limitation in the packaging capacity of scAAV, 30, 300, 500, and 800-1,000 bp of HAs were designed for the right and left side of the Cas9-targeting site to find the most optimal length of HAs and to provide possible lengths of HAs to be chosen based on the size of transgenes by researchers (FIG. 28A). Additionally, due to limitations in packaging capacity compared with ssAAV, scAAV is not suitable for larger transgenes such as CARs targeting CD33. Therefore, based on the size of transgenes, ssAAV and scAAV were both tested and designed, which provides a wide range of options for gene insertion in primary NK cells.

Since designing HAs is a time-consuming procedure and requires multiple optimizations, the CRISPaint approach was also designed, a homology-independent method for gene insertion or tagging. In this method, the same Cas9-targeting site, including the CRISPR RNA (crRNA) and protospacer adjacent motif (PAM) sequence, is provided in the DNA template encoding the gene of interest. Upon the introduction of the Cas9 complex, both template and genomic DNA will be cut simultaneously. As a result, the CRISPaint template is presented as a linearized double-stranded DNA that can be in-tegrated through non-HR machinery (FIG. 28B). The combination of Cas9/RNP and AAV6 gene delivery was also used, and two different human primary CD33CAR NK cells were generated with enhanced anti-AML activity. The feasibility of using Cas9/RNP and AAV to generate CAR-NK cells using two different CAR constructs targeting CD33 is also shown. Differences in the transmembrane domains of the CARs may explain their different surface-expression levels since both CARs are inserted into the same locus with the same promoter, reducing epigenetic variability in expression seen with methods that result in random insertion sites. No significant differences were observed in their anti-AML activity, but the different CAR constructs warrant further investigation to better understand the biologic consequences on expression and effector function in NK cells, which is different from that in T cells. Although successful NK cell transduction has been reported using non-viral and feeder-cell-free approaches, transduction efficiency, silencing of expression, cell death, and obtaining sufficient numbers of NK cells after gene editing continues to be a hurdle. Herein, it is shown that the combination of activation/expansion on IL-21-expressing feeder cells, Cas9/RNP, and AAV resulted in highly efficient CAR expression and production of large numbers of gene-modified NK cells applicable to cancer immunotherapy. This manufacturing method can also serve as a platform to answer broader questions on the utility, design, persistence, and function of CAR-NK cells in screening assays and in vivo models that require large numbers of modified cells. Overall, this method has wide applicability in immunology, cancer immunotherapy, and basic biology of NK cells.

### Materials and Methods

### Cell Lines and Primary Cultures

The irradiated mbIL21-expressing feeder cells (FC21) are genetically modified human female chronic myeloid leukemia cell line K562. Before irradiation, the cells were cultured and grown in RPMI 1640 medium containing 10% fetal bovine serum (FBS) and penicillin/streptomycin, and maintained at 37 C, 5% CO2. Human acute myeloid leukemia cell lines HL-60 (CCL-240™) and Kasumi-1 (CRL-2724™) were purchased from American type culture collection (ATCC). Primary human AML cells (AML-10) were obtained from a pediatric patient with relapsed M5 AML and passaged in NOD-SCID mice. Human primary NK cells were isolated from buffy coats obtained from healthy donor and purchased from Red Cross. The buffy coats were exempt from IRB approval. The stimulated cells were cultured for 7 days in serum-free AIM-V/ICSR expansion medium containing 50 IU/mL of IL-2.

### Human NK Cell Purification and Expansion

NK cells were purified. Briefly, NK cells were isolated from PBMC collected from healthy individuals using RosetteSep™ Human NK Cell Enrichment Cocktail. Purified NK cells were stimulated with irradiated feeder cells (FC21) comprised of K562 transduced with 4-1BBL and membrane-bound IL-21 (mbIL21) at a ratio of 2:1 (feeder:NK).

### ATAC-Seq Assay

Freshly-isolated (naive) and FC21-expanded NK cells from two donors were cryopreserved in aliquots of 100,000 viable cells/vial before processing for ATAC-seq. ATAC-seq was performed. DNA libraries were sequenced using Illumina HiSeq 2500 at 50 bp paired-end reads.

### Cas9/RNP Electroporation for Targeting AAVS1 in NK Cells

AAVS1 was targeted using gRNA (crRNA: 5′GGGGCCACTAGGGACAGGAT (SEQ ID NO: 34)) via electroporation of Cas9/RNP into NK cells seven days after stimulation with FC21. Briefly, 3 3 10⁶ expanded NK cells were harvested and washed twice with 13 mL of PBS followed by centrifugation for 5 min at 400 g and aspiration of PBS. The cell pellet was resuspended in 20 ul of P3 Primary Cell 4D-Nucleofector Solution. 5 ul of pre-complexed Cas9/RNP (Alt-R® CRISPR-Cas9 crRNA, Alt-R® CRISPR-Cas9 tracrRNA, or preassembled synthetic sgRNA (Synthego, Menlo Park, CA) and Alt-R® S.p. HiFi Cas9 Nuclease V3) (Integrated DNA Technologies, Inc., Coralville, Iowa), targeting AAVS1 and 1 ul of 100 μM electroporation enhancer (Alt-R® Cas9 Electroporation Enhancer) were added to the cell suspension. The total volume of 26 ul of CRISPR reaction was transferred into 4D-Nucleofector™ 16-well Strip and electroporated using program EN-138. After electroporation, the cells were transferred into 2 mL of media containing 50 IU of IL-2 in a 12 well plate and incubated at 37 degrees and 5% CO₂ pressure. Two days post electroporation, cells were stimulated with 2 3 10⁶ feeder cells, and 8 mL fresh media complemented with 50 IU was added in cell suspension and kept in a T25 flask.

### Inference of CRISPR Edits (ICE) Mutation Detection Assay

To measure the indel rate in AAVS1KO NK cells, the Cas9/RNP targeted site was PCR amplified using forward and reverse primers described in Tables 1-3. The amplicons were sequenced using Sanger sequencing, and results were analyzed using ICE (Synthego, Menlo Park, CA).

### RNA-Seq Sample Preparation and Sequencing

Total RNA was purified from NK cells after fresh isolation or after 14 days of expansion on FC21, using the Total RNA Purification Plus Kit (Norgen Biotek, ON, Canada). The resulting total RNA was sequenced and analyzed.

### AAV6 Production

The transgenes cloned into ssAAV or scAAV plasmids were packaged in AAV6 capsids. FIGS. 28A and 28B provide the detail of the constructs.

Combining Cas9/RNP and AAV6 to Generate mCherry and CAR NK Cells

A media change and resuspension at 5 3 10⁵ cells per mL was performed on day 6 of NK cell expansion one day before experimental manipulation. The NK cells were electroporated with Cas9/RNP targeting AAVS1 on day 7, as described above. Thirty minutes after electroporation, 3 3 10⁵ live cells were collected and resuspended at 1 3 10⁶ cells per mL in media containing 50 IU IL2 (Novartis) in a 24 well plate in a total volume of 300 ul. For each transduction condition with ssAAV6 or scAAV6 to deliver HDR or CRISPaint DNA encoding mCherry or CD33CARs, we transduced 3 3 10⁵ electroporated cells with 300K MOI (10-500K MOI if needed). Negative controls included as NK cells that were not electroporated, or were electroporated with Cas9/RNP but not AAV transduced, or were transduced with 300K MOI of AAV6 without electroporation of Cas9/RNP. The day after electroporation and transduction, 300 ul of fresh media containing 50 IU of IL2 was added to each well without changing the old media. The cells were kept in culture for 48 h after electroporation and were then re-stimulated with 2 3 10⁶ feeder cells and kept in a total volume of 2 mL media containing 50 IU in 12 well plate, without changing the old media. 48 h later, 8 mL fresh media supplemented with IL2 was added to cells, a total volume of 10 mL was kept in a T25 flask. At day 7 post-trans-duction, cells were re-stimulated with feeder cells at a ratio of 1:1 and grown for one more week, every 2 days fresh media was added to the cells.

### Flow Cytometry for Detection of CAR-NK Cells and Cancer Cells

7 days and 14 days following electroporation, 5 3 10⁵ NK cells were washed twice with staining buffer containing 2% FBS in PBS. Next, 2.5 ug of recombinant human siglec-3/CD33 Fc chimera protein, (CF; R&D systems #1137-SL-050) was added to cell suspension in a total volume of 80 ul and incubated for 30 min at 4 C. Cells were washed twice with staining buffer before staining with 2 ul of Alexa Fluor® 647 affinipure goat anti-human IgG, Fcg fragment specific, (Jackson ImmunoResearch #109-605-098) at 1:100 ratio in 200 μl of staining buffer and kept at 4 C for 30 min. 5 3 10⁵ AML cell lines were resuspended in 50 ul of staining buffer for 30 min in the presence of 2 ul of CD33 antibody. Once stained, cells were washed twice with staining buffer then acquired on MacsQuant flow cytometers. Flow cytometry data were analyzed using FlowJo software (FlowJo, LLC).

### Cytotoxicity Assay

Cytotoxicity assays were performed for 3-4 hours using a calcein-acetoxymethyl-release assay. Cytotoxicity was assessed against Kasumi-1, HL60, or AML10 cells at different effector:target ratios as defined in FIGS. 32A, 32B, 32C, 32D, and 32E.

### CD107a Staining

NK cells and cancer cells were cocultured at 10:1 ratio and supplemented with 20 ul of PE mouse anti-human CD107a antibody (BD Pharmingen™, #555801) in a total volume of 220 ul in a 96 well plate at 37 C incubator for 90 min. The cells were washed with staining buffer once and analyzed on MacsQuant flow cytometer.
Donor 1-Day 9 post Sort
300K MOI-scPAMPAM
Donor 1-Day 9 post Sort
500K MOI-ss800

### Cytokine Secretion Assay

Expanded wildtype or expanded CD33-Gen2 CAR-NK cells from three donors were co-cultured with AML target cells (Kasumi-1) in 96 well plates at a ratio of 1:10. After 4 h, the supernatants were collected and quantified using a multi-plex cytokine assay according the manufacturer's instructions (Bio-Plex Pro Human Cytokine 8-plex Assay, #M50000007A, Bio-Rad) on the Bio-plex 200 system.

### PCR-Based Detection of Transgenes Integration

In-out PCR was performed using 2 pairs of primers (FIGS. 31A and 31B and Tables 1-3) designed inside or outside of the CD33CAR constructs. A set of primers were added to amplify 1200 bp right and left flanking region of Cas9 targeting and transgene integration site (FIG. 31A). PCRs were performed using the Platinum™ Taq DNA polymerase high fidelity kit.

### Targeted Locus Amplification (TLA)

For the whole-genome mapping of CD33CAR-Gen2 integration, we used the TLA technology. The genomic DNA from CD33CAR-expressing NK cells was isolated using Qiagene DNeasy Blood & Tissue Kit and crosslinked, fragmented, and re-ligated using the kit provided by Cergentis, then submitted to Cergentis for sequencing.

### Real-Time Potency Assessment for Suspension Target Cells Killed by WT and CD33CAR NK Cells

The xCELLigence RTCA MP instrument (ACEA Biosciences) was utilized. Briefly, the CD29 tethering reagent was coated on the plate (2 mg/mL, for 3 h at 37° C.) to immobilize the Kasumi cell line, seeded at 6 3 10⁴/well. After 20-24 h, WT or CD33CAR NK cells were added at indicated E:T ratios. An “effector cell only” control was included in the presence of tethering reagent. Cell Index values reflecting viable target cell adherence were applied to xIMT software to plot percentage cytolysis.

### AML-NK Cell Co-Culture

Cells were grown in SFEM II (StemCell, Cambridge, MA) supplemented with the following cytokines; SCF, IL-6, TPO, FLT3, GM-CSF, G-CSF, IL-3 (20 ng/mL) and EPO (10 ng/ml). A de-identified primary AML sample was obtained from OSU Leukemia Tissue Bank consistent with Declaration of Helsinki. NK or CAR-NK cells at a target:effector ratio of 1:2 were cultured for either 6 or 24 h. Following culture, cells were prepared for staining.

### Mass Cytometry Staining and Analysis

Fixed cells were washed with CSM. Samples were resuspended in 1:20 dilution of four elemental equilibration beads (Fluidigm) at a concentration of 1 million cells/mL. FCS generated files were normalized using a normalization tool developed and analyzed on Cytobank (www.cytobank.org).

Once the singlet gate was established, cells were identified using markers and analyzed using SPADE.

Data are presented as mean±standard error of the mean (SEM). Statistical significance was determined GraphPad Prism software using Student's t test, one-way or two-way ANOVA as indicated and where appropriate adjusted for multiple comparison. p values <0.05 were considered statistically significant and indicated by * (p<0.05), ** (p<0.01), or *** (p<0.001). The number of biological replicates including NK cell donors included in each experiment is indicated in the figure legend. Except for the CyTOF analysis, NK cells expressing mCherry, and flow cytometry on cancer cells, all experiments were performed as at least 2 independent experiments. The number of biological and technical replicates for each experiment are indicated in the figures and/or figure legends.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### Tables

TABLE 1
Primers used for interference of CRISPR Edits
(ICE) mutation detection assay

| | |
|---|---|
| Forward primer | 5′-TTCTCCTGTGGATTCGGGTCAC-3′ |
| | (SEQ ID NO: 35) |
| Reverse primer | 5′-CTCTCTGGCTCCATCGTAAGCA-3′ |
| | (SEQ ID NO: 36) |

TABLE 2
Primers used for PCR-based detection of
transgenes integration
Condition 1

| | |
|---|---|
| Reverse-1200 bp (1) | TCCTGGGCAAACAGCATAA |
| | (SEQ ID NO: 37) |
| Forward-CD33CAR (1) | GAGCTGCAGAAGGACAAGAT |
| | (SEQ ID NO: 38) |

Condition 2

| | |
|---|---|
| Reverse-CD33CAR (2) | CTCGTGTCATCTGGATGTCTG |
| | (SEQ ID NO: 39) |
| Forward-1200 bp (2) | CTTTGAGCTCTACTGGCTTCTG |
| | (SEQ ID NO: 40) |

Condition 3

| | |
|---|---|
| Reverse-1200 bp (1) | TCCTGGGCAAACAGCATAA |
| | (SEQ ID NO: 37) |
| Forward-1200 bp (2) | CTTTGAGCTCTACTGGCTTCTG |
| | (SEQ ID NO: 40) |

TABLE 3
Vector-specific primer sets used for targeted
locus amplification (TLA).

| | | | |
|---|---|---|---|
| 1 | 600bp HA AAVS1 | Reverse | GCGAGTGAAGACGGCATG |
| | | | (SEQ ID NO: 41) |
| | | Forward | GTCTGTGCTAGCTCTTCCAG |
| | | | (SEQ ID NO: 42) |
| 2 | CD33CAR-Gen2 | Reverse | GCGATGTCAGAAGGGTAAA |
| | | | (SEQ ID NO: 43) |
| | | Forward | GGCGGACACTCTGACTACAT |
| | | | (SEQ ID NO: 44) |

TABLE 4
Transgene sequence variants identified.

| | | | | Primer | | Primer | |
|---|---|---|---|---|---|---|---|
| | | | | Set 1 | | Set 2 | |
| Region | Position | Reference | Mutation | Coverage | % | Coverage | % |
| 600 bp | 4,116 | Thymine | Cytosine | 193 | 37 | 341 | 14 |
| | | (T) | (C) | | | | |

TABLE 5
The construct to be used for gene insertion in AAVS1 locus using AAV vectors
between the ITRs.

| Name of the | |
|---|---|
| sequence | Sequence |
| gRNA targeting | GGGGCCACTAGGGACAGGATTGG |
| AAVS1 + PAM | (SEQ ID NO: 45) |
| (For CRISPaint | |
| construct) | |
| Spacer and Cloning | GCGGCCGGCGCGCC (SEQ ID NO: 46) |
| site AscI | |
| 800 bp | TGCTTTCTCTGACCTGCATTCTCTCCCCTGGGCCTGTGCCGCTT |
| Homology arm | TCTGTCTGCAGCTTGTGGCCTGGGTCACCTCTACGGCTGGCCC |
| Right | AGATCCTTCCCTGCCGCCTCCTTCAGGTTCCGTCTTCCTCCACT |
| | CCCTCTTCCCCTTGCTCTCTGCTGTGTTGCTGCCCAAGGATGCT |
| | CTTTCCGGAGCACTTCCTTCTCGGCGCTGCACCACGTGATGTCC |
| | TCTGAGCGGATCCTCCCCGTGTCTGGGTCCTCTCCGGGCATCTC |
| | TCCTCCCTCACCCAACCCCATGCCGTCTTCACTCGCTGGGTTCC |
| | CTTTTCCTTCTCCTTCTGGGGCCTGTGCCATCTCTCGTTTCTTAG |
| | GATGGCCTTCTCCGACGGATGTCTCCCTTGCGTCCCGCCTCCCC |
| | TTCTTGTAGGCCTGCATCATCACCGTTTTTCTGGACAACCCCAA |
| | AGTACCCCGTCTCCCTGGCTTTAGCCACCTCTCCATCCTCTTGC |
| | TTTCTTTGCCTGGACACCCCGTTCTCCTGTGGATTCGGGTCACC |
| | TCTCACTCCTTTCATTTGGGCAGCTCCCCTACCCCCCTTACCTC |
| | TCTAGTCTGTGCTAGCTCTTCCAGCCCCCTGTCATGGCATCTTC |
| | CAGGGGTCCGAGAGCTCAGCTAGTCTTCTTCCTCCAACCCGGG |
| | CCCCTATGTCCACTTCAGGACAGCATGTTTGCTGCCTCCAGGG |
| | ATCCTGTGTCCCCGAGCTGGGACCACCTTATATTCCCAGGGCC |
| | GGTTAATGTGGCTCTGGTTCTGGGTACTTTTATCTGTCCCCTCC |
| | ACCCCACAGTGGGGC (SEQ ID NO: 47) |
| Spacer between | CACTAGGGACAGCGATCGGGTAC (SEQ ID NO: 48) |
| Homology arm and | |
| promoter | |
| Splice Acceptor | ATCGATCGCAGGCGCAATCTTCGCATTTCTTTTTTCCAG |
| | (SEQ ID NO: 49) |
| MND promoter | ACGGGGTTGGACGCGTCTTAATTAAGGATCCAAGGTCAGGAA |
| (This promoter | ATCGATCACGAGACTAGCCTCGAGAAGCTTGATATCGAATTCC |
| should be used | CAGAGAAACAGGAGAATATGGGCCAAACAGGATATCTGTGGT |
| instead of Splice | AAGCAGTTCCTGCCCCGGCTCAGGGCCAAGAACAGTTGGAAC |
| Acceptor if a large | AGCAGAATATGGGCCAAACAGGATATCTGTGGTAAGCAGTTC |
| transgene needs to | CTGCCCCGGCTCAGGGCCAAGAACAGATGGTCCCCAGATGCG |
| be expressed). | GTCCCGCCCTCAGCAGTTTCTAGAGAACCATCAGATGTTTCCA |
| | GGGTGCCCCAAGGACCTGAAATGACCCTGTGCCTTATTTGAAC |
| | TAACCAATCAGTTCGCTTCTCGCTTCTGTTCGCGCGCTTCTGCT |
| | CCCCGAGCTCTATATAAGCAGAGCTCGTTTAGTGAACCGTCAG |
| | ATCGCCTGGAGACGCCATCCACGCTGTTTTGACCTCCATAGAA |
| | GACACCGACTCTAGAGGATCGATCCCCCGGGCTGCAGGAATT |
| | CAAGCGAGAAGACAAGGGCAGAAAG CACC (SEQ ID NO: 50) |
| Vertebrate | GCCACC |
| consensus | |
| sequence for | |
| strong initiation of | |
| translation (Kozak | |
| 1987) must be used | |
| before start codon | |
| of the transgene if | |
| MND | |
| promoter is used. | |
| (No need to use, if | |
| the Splice | |
| Acceptor is used) | |
| Transgene | GTGAGCAAGGGCGAGGAGGATAACATGGCCATCATCAAGGAG |
| mCherry | TTCATGCGCTTCAAGGTGCACATGGAGGGCTCCGTGAACGGCC |
| | ACGAGTTCGAGATCGAGGGCGAGGGCGAGGGCCGCCCCTACG |
| | AGGGCACCCAGACCGCCAAGCTGAAGGTGACCAAGGGTGGCC |
| | CCCTGCCCTTCGCCTGGGACATCCTGTCCCCTCAGTTCATGTAC |
| | GGCTCCAAGGCCTACGTGAAGCACCCCGCCGACATCCCCGACT |
| | ACTTGAAGCTGTCCTTCCCCGAGGGCTTCAAGTGGGAGCGCGT |
| | GATGAACTTCGAGGACGGCGGCGTGGTGACCGTGACCCAGGA |
| | CTCCTCCCTGCAGGACGGCGAGTTCATCTACAAGGTGAAGCTG |
| | CGCGGCACCAACTTCCCCTCCGACGGCCCCGTAATGCAGAAGA |
| | AGACCATGGGCTGGGAGGCCTCCTCCGAGCGGATGTACCCCGA |
| | GGACGGCGCCCTGAAGGGCGAGATCAAGCAGAGGCTGAAGCT |
| | GAAGGACGGCGGCCACTACGACGCTGAGGTCAAGACCACCTA |
| | CAAGGCCAAGAAGCCCGTGCAGCTGCCCGGCGCCTACAACGT |
| | CAACATCAAGTTGGACATCACCTCCCACAACGAGGACTACACC |
| | ATCGTGGAACAGTACGAACGCGCCGAGGGCCGCCACTCCACC |
| | GGCGGCATGGACGAGCTGTACAAGTAA (SEQ ID NO: 51) |
| CD33CAR-Gen2 | ATGCTGCTGCTGGTGACCTCTCTGCTGCTGTGCGAGCTGCCAC |
| | ACCCAGCCTTCCTGCTGATCCCAGACATCCAGATGACACAGAG |
| | CCCCAGCTCCCTGAGCGCCTCCGTGGGCGACAGAGTGACCATC |
| | ACATGTAGGGCCTCTGAGAGCGTGGATAACTATGGCATCAGCT |
| | TCATGAATTGGTTTCAGCAGAAGCCTGGCGGCGCCCCAAAGCT |
| | GCTGATCTACGCAGCCAGCATGCAGGGCTCCGGCGTGCCCTCT |
| | CGGTTCTCCGGCTCTGGCAGCGGCACCGACTTCACCCTGACAA |
| | TCTCTAGCCTGCAGCCAGACGATTTCGCCACATACTATTGCCA |
| | GCAGAGCAAGGAGGTGCCCTGGACCTTTGGCCAGGGCACAAA |
| | GGTGGAGATCAAGGGCTCCACCTCTGGCAGCGGCAAGCCTGG |
| | CAGCGGAGAGGGCTCCACAAAGGGACAGGTGCAGCTGGTGCA |
| | GTCCGGAGCCGAGGTGAAGAAGCCAGGCTCCTCTGTGAAGGT |
| | GTCTTGTAAGGCCAGCGGCTATACCTTCACAGACTACAACATG |
| | CACTGGGTGCGCCAGGCACCAGGACAGGGCCTGGAGTGGATC |
| | GGCTACATCTATCCTTACAACGGCGGCACCGGCTATAATCAGA |
| | AGTTTAAGTCCAAGGCCACCATCACAGCCGATGAGTCTACCAA |
| | TACAGCCTACATGGAGCTGAGCAGCCTGCGGTCCGAGGACAC |
| | AGCCGTGTACTATTGCGCCCGGGGCAGACCCGCTATGGACTAT |
| | TGGGGCCAGGGCACCCTGGTGACAGTGTCTAGCGAGAGCAAG |
| | TACGGACCACCTTGCCCACCATGTCCTGCACCAGAGTTCCTGG |
| | GAGGACCTTCCGTGTTCCTGTTTCCTCCAAAGCCAAAGGACAC |
| | CCTGATGATCAGCCGGACCCCAGAGGTGACATGCGTGGTGGTG |
| | GACGTGAGCCAGGAGGACCCCGAGGTGCAGTTCAACTGGTAC |
| | GTGGATGGCGTGGAGGTGCACAATGCCAAGACCAAGCCAAGA |
| | GAGGAGCAGTTTAACTCCACCTATAGGGTGGTGTCTGTGCTGA |
| | CAGTGCTGCACCAGGACTGGCTGAACGGCAAGGAGTACAAGT |
| | GCAAGGTGTCCAATAAGGGCCTGCCTTCCTCTATCGAGAAGAC |
| | CATCTCTAAGGCAAAGGGACAGCCAAGGGAGCCACAGGTGTA |
| | TACACTGCCCCCTAGCCAGGAGGAGATGACCAAGAACCAGGT |
| | GTCCCTGACATGTCTGGTGAAGGGCTTTTACCCTTCTGACATCG |
| | CCGTGGAGTGGGAGAGCAATGGCCAGCCAGAGAACAATTATA |
| | AGACCACACCACCCGTGCTGGACTCTGATGGCAGCTTCTTTCT |
| | GTACAGCCGCCTGACCGTGGATAAGTCCCGGTGGCAGGAGGG |
| | CAACGTGTTCTCCTGCTCTGTGATGCACGAGGCCCTGCACAAT |
| | CACTACACACAGAAGAGCCTGTCCCTGTCTCTGGGCAAGATGT |
| | TTTGGGTGCTGGTGGTGGTGGGAGGCGTGCTGGCCTGTTATTC |
| | CCTGCTGGTGACCGTGGCCTTCATCATCTTTTGGGTGCGCTCCA |
| | AGCGGAGCCGGGGCGGACACTCTGACTACATGAACATGACCC |
| | CACGGAGACCCGGACCTACAAGGAAGCACTATCAGCCCTACG |
| | CCCCTCCACGGGACTTCGCAGCATATCGCAGCCGGGTGAAGTT |
| | TAGCAGATCCGCCGATGCACCAGCATATCAGCAGGGACAGAA |
| | TCAGCTGTACAACGAGCTGAATCTGGGCAGGCGCGAGGAGTA |
| | CGACGTGCTGGATAAGAGGCGGGGCCGGGACCCCGAGATGGG |
| | AGGCAAGCCCAGGCGCAAGAACCCTCAGGAGGGCCTGTATAA |
| | TGAGCTGCAGAAGGACAAGATGGCCGAGGCCTACAGCGAGAT |
| | CGGCATGAAGGGAGAGCGGAGAAGGGGCAAGGGACACGATG |
| | GCCTGTATCAGGGCCTGTCCACCGCCACAAAGGACACCTACGA |
| | TGCACTGCACATGCAGGCCCTGCCACCTCGGTGA |
| | (SEQ ID NO: 52) |
| CD33CAR- | ATGCTGCTGCTGGTGACCTCCCTGCTGCTGTGCGAGCTGCCAC |
| Gen4v2 | ACCCTGCCTTTCTGCTGATCCCAGACATCCAGATGACACAGAG |
| | CCCCAGCTCCCTGTCTGCCAGCGTGGGCGACAGAGTGACCATC |
| | ACATGTAGGGCCTCCGAGTCTGTGGATAACTATGGCATCAGCT |
| | TTATGAATTGGTTCCAGCAGAAGCCAGGAGGCGCCCCTAAGCT |
| | GCTGATCTACGCAGCCTCCATGCAGGGCTCTGGCGTGCCCAGC |
| | CGCTTTAGCGGCTCCGGCTCTGGCACCGATTTCACCCTGACAA |
| | TCTCTAGCCTGCAGCCAGACGATTTTGCCACATACTATTGCCA |
| | GCAGTCCAAGGAGGTGCCCTGGACCTTCGGCCAGGGCACAAA |
| | GGTGGAGATCAAGGGCAGCACCTCCGGCTCTGGCAAGCCTGG |
| | CTCCGGAGAGGGCTCTACAAAGGGACAGGTGCAGCTGGTGCA |
| | GAGCGGAGCCGAGGTGAAGAAGCCAGGCTCCTCTGTGAAGGT |
| | GAGCTGTAAGGCCTCCGGCTATACCTTTACAGACTACAACATG |
| | CACTGGGTGAGACAGGCACCAGGACAGGGCCTGGAGTGGATC |
| | GGCTACATCTATCCTTACAACGGCGGCACCGGCTATAATCAGA |
| | AGTTCAAGAGCAAGGCCACCATCACAGCCGATGAGTCCACCA |
| | ATACAGCCTACATGGAGCTGAGCAGCCTGAGGAGCGAGGACA |
| | CAGCCGTGTACTATTGCGCCAGAGGCAGGCCTGCTATGGACTA |
| | TTGGGGCCAGGGCACCCTGGTGACAGTGTCTAGCGAGTCCAAG |
| | TACGGACCACCTTGCCCACCATGTCCAGCACCAGAGTTTCTGG |
| | GAGGACCTAGCGTGTTTCTGTTCCCTCCAAAGCCAAAGGACAC |
| | CCTGATGATCAGCAGAACCCCCGAGGTGACATGCGTGGTGGTG |
| | GACGTGTCCCAGGAGGACCCCGAGGTGCAGTTTAACTGGTACG |
| | TGGATGGCGTGGAGGTGCACAATGCCAAGACCAAGCCTAGAG |
| | AGGAGCAGTTCAACTCCACCTATAGGGTGGTGTCTGTGCTGAC |
| | AGTGCTGCACCAGGACTGGCTGAACGGCAAGGAGTACAAGTG |
| | CAAGGTGTCTAATAAGGGCCTGCCATCCTCTATCGAGAAGACC |
| | ATCAGCAAGGCCAAGGGCCAGCCTAGGGAGCCACAGGTGTAT |
| | ACACTGCCCCCTTCCCAGGAGGAGATGACCAAGAACCAGGTGT |
| | CTCTGACATGTCTGGTGAAGGGCTTCTACCCATCCGACATCGC |
| | CGTGGAGTGGGAGTCTAATGGCCAGCCCGAGAACAATTATAA |
| | GACCACACCACCCGTGCTGGACTCTGATGGCAGCTTCTTTCTGT |
| | ACTCTCGCCTGACCGTGGATAAGAGCCGGTGGCAGGAGGGCA |
| | ACGTGTTTAGCTGCTCCGTGATGCACGAGGCCCTGCACAATCA |
| | CTACACACAGAAGTCTCTGAGCCTGTCCCTGGGCAAGAGCAAC |
| | CTGTTCGTGGCCTCCTGGATCGCCGTGATGATCATCTTTCGCAT |
| | CGGCATGGCCGTGGCCATCTTCTGCTGTTTCTTTTTCCCATCCG |
| | GAGGCTCTGGAGGAGGCTCCGGCTGGCGGAGAAAGCGGAAGG |
| | AGAAGCAGAGCGAGACCTCCCCTAAGGAGTTTCTGACAATCTA |
| | TGAGGACGTGAAGGATCTGAAGACCAGGCGCAATCACGAGCA |
| | GGAGCAGACCTTCCCAGGAGGAGGCTCTACAATCTACAGCATG |
| | ATCCAGTCCCAGAGCAGCGCCCCAACCAGCCAGGAGCCAGCC |
| | TATACACTGTACTCTCTGATCCAGCCTAGCCGGAAGTCTGGCA |
| | GCCGCAAGCGGAACCACTCCCCATCTTTCAATTCTACCATCTAT |
| | GAAGTGATCGGCAAGAGCCAGCCTAAGGCCCAGAACCCAGCC |
| | AGACTGTCCAGGAAGGAGCTGGAGAATTTTGACGTGTACTCTG |
| | GAGGCAGCGGAGGAGGCTCTGGCCGCGTGAAGTTCAGCCGGT |
| | CCGCCGATGCCCCAGCCTATAAGCAGGGCCAGAACCAGCTGTA |
| | CAACGAGCTGAATCTGGGCCGGAGAGAGGAGTACGACGTGCT |
| | GGATAAGAGGCGGGGCCGGGACCCCGAGATGGGAGGCAAGCC |
| | CCGGAGAAAGAACCCTCAGGAGGGCCTGTATAATGAGCTGCA |
| | GAAGGACAAGATGGCCGAGGCCTACTCCGAGATCGGCATGAA |
| | GGGAGAGAGGCGCCGGGGCAAGGGACACGATGGCCTGTATCA |
| | GGGCCTGAGCACCGCCACAAAGGACACCTACGATGCCCTGCA |
| | CATGCAGGCCCTGCCTCCACGGTGATGA (SEQ ID NO: 53) |
| Spacer between the | AAGTAACGC |
| stop codon of the | |
| transgene and | |
| BGHpA | |
| BGHpA | CCTCGACTGTGCCTTCTAGTTGCCAGCCATCTGTTGTTTGCCCC |
| | TCCCCCGTGCCTTCCTTGACCCTGGAAGGTGCCACTCCCACTGT |
| | CCTTTCCTAATAAAATGAGGAAATTGCATCGCATTGTCTGAGT |
| | AGGTGTCATTCTATTC |
| | (SEQ ID NO: 54) |
| 1000 bp | GATTGGTGACAGAAAAGCCCCATCCTTAGGCCTCCTCCTTCCT |
| Homology arm | AGTCTCCTGATATTGGGTCTAACCCCCACCTCCTGTTAGGCAG |
| left | ATTCCTTATCTGGTGACACACCCCCATTTCCTGGAGCCATCTCT |
| | CTCCTTGCCAGAACCTCTAAGGTTTGCTTACGATGGAGCCAGA |
| | GAGGATCCTGGGAGGGAGAGCTTGGCAGGGGGTGGGAGGGAA |
| | GGGGGGGATGCGTGACCTGCCCGGTTCTCAGTGGCCACCCTGC |
| | GCTACCCTCTCCCAGAACCTGAGCTGCTCTGACGCGGCTGTCT |
| | GGTGCGTTTCACTGATCCTGGTGCTGCAGCTTCCTTACACTTCC |
| | CAAGAGGAGAAGCAGTTTGGAAAAACAAAATCAGAATAAGTT |
| | GGTCCTGAGTTCTAACTTTGGCTCTTCACCTTTCTAGTCCCCAA |
| | TTTATATTGTTCCTCCGTGCGTCAGTTTTACCTGTGAGATAAGG |
| | CCAGTAGCCAGCCCCGTCCTGGCAGGGCTGTGGTGAGGAGGG |
| | GGGTGTCCGTGTGGAAAACTCCCTTTGTGAGAATGGTGCGTCC |
| | TAGGTGTTCACCAGGTCGTGGCCGCCTCTACTCCCTTTCTCTTT |
| | CTCCATCCTTCTTTCCTTAAAGAGTCCCCAGTGCTATCTGGGAC |
| | ATATTCCTCCGCCCAGAGCAGGGTCCCGCTTCCCTAAGGCCCT |
| | GCTCTGGGCTTCTGGGTTTGAGTCCTTGGCAAGCCCAGGAGAG |
| | GCGCTCAGGCTTCCCTGTCCCCCTTCCTCGTCCACCATCTCATG |
| | CCCCTGGCTCTCCTGCCCCTTCCCTACAGGGGTTCCTGGCTCTG |
| | CTCTTCAGACTGAGCCCCGTTCCCCTGCATCCCCGTTCCCCTGC |
| | ATCCCCCTTCCCCTGCATCCCCCAGAGGCCCCAGGCCACCTAC |
| | TTGGCCTGGACCCCACGAGAGGCCACCCCAGCCCTGTCTACCA |
| | GGCTGCCTTTTGGGTGGATTCTCCTCCAACTGTGGGGTGACTGC |
| | TTGG (SEQ ID NO: 55) |
| Not1 cloning site | GCGGCCGC |

TABLE 6
Identification of Structural Variants. The TLA analysis identified
four vector-vector breakpoints that represent concatemerization of
multiple copies of the vector and/or structural rearrangements in a
single vector sequence. All fusions were located at the annotated
homology arm. Due to the heterogeneous nature of the sample, it is
expected that these fusions are only present in a subset of the
sample. It should be noted that three out of four fusions show 9-12
bp homology which might indicate technical bias.

Vector: 149 (head) fused to Vector: 4116 (tail) (in bold) with 9
homologous bases (in italics)
GGCATGGGGTTGGGTGAGGGAGGAGAGATGCCCGGAGAGGACCCAGACACGGGG
AGGATCCGCTCAGAGGACATCACGTGGTGCAGCGGCGCGCCGGCCGCAGAAAGGG
AGTAGAGGCGGCCACGACCTGGTGAACACCTAGGACGCACCATTCTCACAAA
GGGAGTTTTCCACACGGACACCCCCCTCCTCACCACAGCCCTGCCAGGACGG
GGCTGGCTACTGGCCTTATCTC (SEQ ID NO: 56)
Vector: 149 (head) fused to Vector: 4,113 (tail) (in bold) with 12
homologous bases (in italics)
GCGAGTGAAGACGGCATGGGGTTGGGTGAGGGAGGAGAGATGCCCGGAGAGGACCC
AGACACGGGGAGGATCCGCTCAGAGGACATCACGTGGTGCAGCGGCGCGCCGGCCG
CAGGAAGGGAGTAGAGGCGGCCACGACCTGGTGAACACCTAGGACGCACCATTC
TCACAAAGGGAGTTTTCCACACGGACACCCCCCTCCTCACCACAGCCCTGCCAG
GACGGGGCTGGCTACTGGCCTTA (SEQ ID NO: 57)
Vector: 155 (head) fused to Vector: 4,163 (tail) (in bold) with 6
homologous bases (in italics)
GCGAGTGAAGACGGCATGGGGTTGGGTGAGGGAGGAGAGATGCCCGGAGAGGACCC
AGACACGGGGAGGATCCGCTCAGAGGACATCACGTGGTGCAGCGGCGCGCAGAGAG
GGAGTGGCCAACTCCATCACTAGGGGTTCCTGCGGCCGCAGAAAGGGAGTAGA
GGCGGCCACGACCTGGTGAACACCTAGGACGCACCATTCTCACAAAGGGAGTT
TTCCACACGGA (SEQ ID NO: 58)
Vector: 158 (head) fused to Vector: 4,121 (tail) (in bold) with 4
homologous bases (in italics)
GCGAGTGAAGACGGCATGGGGTTGGGTGAGGGAGGAGAGATGCCCGGAGAGGACCC
AGACACGGGGAGGATCCGCTCAGAGGACATCACGTGGTGCAGCGGCCGCAGAAAG
GGAGTAGAGGCGGCCACGACCTGGTGAACACCTAGGACGCACCATTCTCACAA
AGGGAGTTTTCCACACGGACACCCCCCTCCTCACCACAGCCCTGCCAGGACGG
GGCTGGCTACTGGCCTT (SEQ ID NO: 59)

SEQUENCES

1. SEQ ID NO: 1
QVQLQQPGAEVVKPGASVKMSCKASGYTFTSYYIHWIKQTPGQGLEWVGVIYPGNDDISY
NQKFKGKATLTADKSSTTAYMQLSSLTSEDSAVYYCAREVRLRYFDVWGAGTTVTVSS
2. SEQ ID NO: 2
QVQLQQPGAEVVKPGASVKMSCKASGYTFTSYYIHWIKQTPGQGLEWVGVIYPGNDDISY
NQKFQGKATLTADKSSTTAYMQLSSLTSEDSAVYYCAREVRLRYFDVWGQGTTVTVSS
3. SEQ ID NO: 3
QVQLVQSGAEVKKPGSSVKVSCKASGYTFTDYNMHWVRQAPGQGLEWIGYIYPYNGGTG
YNQKFKSKATITADESTNTAYMELSSLRSEDTAVYYCARGRPAMDYWGQGTLVTVSS
4. SEQ ID NO: 4
QVQLVQSGAEVKKPGSSVKVSCKASGYTFTDYNMHWVRQAPGQGLEWIGYIYPYNGGTG
YNQKFKSKATITADESTNTAYMELSSLRSEDTAVYYCARGRPAMDYWGQGTLVTVSS
5. SEQ ID NO: 5
QVQLVQSGAEVKKPGSSVKVSCKASGYTFTDYNMHWVRQAPGQGLEWIGYIYPYNGGTG
YNQKFKSKATITADESTNTAYMELSSLRSEDTAVYYCARGRPAMDYYGQGTLVTVSS
6. SEQ ID NO: 6
QVQLVQSGAEVKKPGSSVKVSCKASGYTWTDYNMHWVRQAPGQGLEWIGYIYPYNGGT
GYNQKFKSKATITADESTNTAYMELSSLRSEDTAVYYCARGRPAMDYWGQGTLVTVSS
7. SEQ ID NO: 7
MLTQSPSSLAVSAGEKVTMSCKSSQSVFFSSSQKNYLAWYQQIPGQSPKLLIYWASTRESG
VPDRFTGSGSGTDFTLTISSVQSEDLAIYYCHQYLSSRTFGGGTKLEIKR
8. SEQ ID NO: 8
EIVLTQSPGSLAVSPGERVTMSCKSSQSVFFSSSQKNYLAWYQQIPGQSPRLLIYWASTRES
GVPDRFTGSGSGTDFTLTISSVQPEDLAIYYCHQYLSSRTFGQGTKLEIKR
9. SEQ ID NO: 9
DIQMTQSPSSLSASVGDRVTITCRASESVDNYGISFMNWFQQKPGGAPKLLIYAASMQGSG
VPSRFSGSGSGTDFTLTISSLQPDDFATYYCQQSKEVPWTFGQGTKVEIK
10. SEQ ID NO: 10
DIQMTQSPSSLSASVGDRVTITCRASESVDNYGISFMNWFQQKPGKAPKLLIYAASNQGSG
VPSRFSGSGSGTDFTLTISSLQPDDFATYYCQQSKEVPWTFGQGTKVEIK
11. SEQ ID NO: 11
DIQMTQSPSSLSASVGDRVTITCRASESVDNYGISFMNWFQQKPGKAPKLLIYAASNQGSG
VPSRFSGSGSGTDFTLNISSLQPDDFATYYCQQSKEVPWTFGQGTKVEIK
12. SEQ ID NO: 12
DIQMTQSPSSLSASVGDRVTITCRASESVDNYGISFMNWFQQKPGKAPKLLIYAASNQGSG
VPSRFSGSGSGTDFTLTISSLQPDDFATYYCQQSKEVPWTFGQGTKVEIK
13. SEQ ID NO: 13-Whitlow Linker
GSTSGSGKPGSGEGSTKG
14. SEQ ID NO: 14-Hu195
QVQLVQSGAEVKKPGSSVKVSCKASGYTFTDYNMHWVRQAPGQGLEWIGYTYPYNGGTG
YNCIKFKSKATITADESTNTAYMELSSLRSEDTAVYYCARGRPAMDYWGQGTLVTVSSGG
GGSGGGGSGGGGSDIQMTQSPSSLSASVGDRVTITCRASESVDNYGISFMNWFQQKPGKAP
KLLIYAASNQGSGVPSRESGSGSGTDFTLTISSLQPDDFATYYCQQSKEVPWTFGQGTKVEI
KTSSG
15. SEQ ID NO: 15-M195
QVQLVQSGAEVKKPGSSVKVSCKASGYTFTDYNMHWVRQAPGQGLEWIGYIYPYNGGTG
YNCIKFKSKATITADESTNTAYMELSSLRSEDTAVYYCARGRPAMDYWGQGTLVTVSSGG
GGSGGGGSGGGGSDIQMTQSPSSLSASVGDRVTITCRASESVDNYGISFMNWFQQKPGKAP
KLLIYAASNQGSGVPSRFSGSGSGTDFTLNISSLQPDDFATYYCQQSKEVPWTFGQGTKVEI
KTSSG
16. SEQ ID NO: 16-CDR
DYNMH
17. SEQ ID NO: 17-CDR
YIYPYNGGTGYNQKFKSKA
18. SEQ ID NO: 18-CDR
GRPAMDYWGQ
19. SEQ ID NO: 19-CDR
RASESVDNYGISFMN
20. SEQ ID NO: 20-CDR
AASNQGS
21. SEQ ID NO: 21-CDR
QQSKEVPWT
22. SEQ ID NO: 22-Mylo
EVOLVQSGAEVKKPGSSVKVSCKASGYTITDSNIHWVRQAPGQSLEWIGYIYPYNGGTDY
NOKFKNRATLTVDNPTNTAYMELSSLRSEDTAFYYCVNGNPWLAYWGQGTLVTVSSGGG
GSGGGGSGGGGSDIQLTQSPSTLSASVGDRVTITCRASESLDNYGIRFLTWFQQKPGKAPKL
LMYAASNQGSGVPSRFSGSGSGTEFTLTISSLQPDDFATYYCQQTKEVPWSFGQGTKVEVK
R
23. SEQ ID NO: 23-CDR
GYTITDSN
24. SEQ ID NO: 24-CDR
IYPYNGGT
25. SEQ ID NO; 25-CDR
VNGNPWLAY
26. SEQ ID NO: 26-CDR
ESLDNYGIRF
27. SEQ ID NO: 27-CDR
QQTKEVPWS
28. SEQ ID NO: 28-Example of AAVS1 KO Indel (0)
CCCCTCCACCCCACAGTGGGGCCACTAGGGACAGGATTGGTGACAGAAAAGCCCCATC
CTTAGGC
29. SEQ ID NO: 29-Example of AAVSI KO Indel (−1)
CCCCTCCACCCCACAGTGGGGCCACTAGGGACAGATTGGTGACAGAAAAGCCCCATCC
TTAGGC
30. SEQ ID NO: 30-Example of AAVSI KO Indel (−4)
CCCCTCCACCCCACAGTGGGGCCACTAGGGAATTGGTGACAGAAAAGCCCCATCCTTA
GGC
31. SEQ ID NO: 31-Example of AAVSI KO Indel (−21)
CCCCTCCACCCCACATTGGTGACAGAAAAGCCCCATCCTTAGGC
32. SEQ ID NO: 32-Example of AAVSI KO Indel (−9)
CCCCTCCACCCCACAGTGGGGCCACGATTGGTGACAGAAAAGCCCCATCCTTAGGC
33. SEQ ID NO: 33-Reference Wildtype
CCCCTCCACCCCACAGT
34. SEQ ID NO: 34-crRNA target sequence
GGGGCCACTAGGGACAGGAT
35. SEQ ID NO: 35-Forward Primer
TTCTCCTGTGGATTCGGGTCAC
36. SEQ ID NO: 36-Reverse Primer
CTCTCTGGCTCCATCGTAAGCA
37. SEQ ID NO: 37-Reverse-1200 bp (1)
TCCTGGGCAAACAGCATAA
38. SEQ ID NO: 38-Forward CD33CAR (1)
GAGCTGCAGAAGGACAAGAT
39. SEQ ID NO: 39-CD33CAR (2)
CTCGTGTCATCTGGATGTCTG
40. SEQ ID NO: 40-1200 bp (1)
CTTTGAGCTCTACTGGCTTCTG
41. SEQ ID NO: 41-600 bp HA AAVSI Reverse
GCGAGTGAAGACGGCATG
42. SEQ ID NO: 42-600 bp HA AAVS1 Forward
GTCTGTGCTAGCTCTTCCAG
43. SEQ ID NO: 43-CD33CAR-Gen2 Reverse
GCGATGTCAGAAGGGTAAA
44. SEQ ID NO: 44-CD33CAR-Gen2 Forward
GGCGGACACTCTGACTACAT
45. SEQ ID NO: 45-gRNA targeting AAVS1 + PAM (For CRISPaint construct)
GGGGCCACTAGGGACAGGATTGG
46. SEQ ID NO: 46-Spacer and Cloning site AscI
GCGGCCGGCGCGCC
47. SEQ ID NO: 47-800 bp Right Homology arm
TGCTTTCTCTGACCTGCATTCTCTCCCCTGGGCCTGTGCCGCTTTCTGTCTGCAGCTTGTG
GCCTGGGTCACCTCTACGGCTGGCCCAGATCCTTCCCTGCCGCCTCCTTCAGGTTCCGTC
TTCCTCCACTCCCTCTTCCCCTTGCTCTCTGCTGTGTTGCTGCCCAAGGATGCTCTTTCCG
GAGCACTTCCTTCTCGGCGCTGCACCACGTGATGTCCTCTGAGCGGATCCTCCCCGTGTC
TGGGTCCTCTCCGGGCATCTCTCCTCCCTCACCCAACCCCATGCCGTCTTCACTCGCTGG
GTTCCCTTTTCCTTCTCCTTCTGGGGCCTGTGCCATCTCTCGTTTCTTAGGATGGCCTTCT
CCGACGGATGTCTCCCTTGCGTCCCGCCTCCCCTTCTTGTAGGCCTGCATCATCACCGTTT
TTCTGGACAACCCCAAAGTACCCCGTCTCCCTGGCTTTAGCCACCTCTCCATCCTCTTGC
TTTCTTTGCCTGGACACCCCGTTCTCCTGTGGATTCGGGTCACCTCTCACTCCTTTCATTT
GGGCAGCTCCCCTACCCCCCTTACCTCTCTAGTCTGTGCTAGCTCTTCCAGCCCCCTGTC
ATGGCATCTTCCAGGGGTCCGAGAGCTCAGCTAGTCTTCTTCCTCCAACCCGGGCCCCTA
TGTCCACTTCAGGACAGCATGTTTGCTGCCTCCAGGGATCCTGTGTCCCCGAGCTGGGAC
CACCTTATATTCCCAGGGCCGGTTAATGTGGCTCTGGTTCTGGGTACTTTTATCTGTCCCC
TCCACCCCACAGTGGGGC
48. SEQ ID NO: 48-Spacer between Homology arm and promoter
CACTAGGGACAGCGATCGGGTAC
49. SEQ ID NO: 49-Splice Acceptor
ATCGATCGCAGGCGCAATCTTCGCATTTCTTTTTTCCAG
50. SEQ ID NO: 50-MND promoter (This promoter should be used instead of
Splice Acceptor if a large transgene needs to be expressed).
ATCGATCACGAGACTAGCCTCGAGAAGCTTGATATCGAATTCCACGGGGTTGGACGCGT
CTTAATTAAGGATCCAAGGTCAGGAACAGAGAAACAGGAGAATATGGGCCAAACAGGA
TATCTGTGGTAAGCAGTTCCTGCCCCGGCTCAGGGCCAAGAACAGTTGGAACAGCAGAA
TATGGGCCAAACAGGATATCTGTGGTAAGCAGTTCCTGCCCCGGCTCAGGGCCAAGAAC
AGATGGTCCCCAGATGCGGTCCCGCCCTCAGCAGTTTCTAGAGAACCATCAGATGTTTC
CAGGGTGCCCCAAGGACCTGAAATGACCCTGTGCCTTATTTGAACTAACCAATCAGTTC
GCTTCTCGCTTCTGTTCGCGCGCTTCTGCTCCCCGAGCTCTATATAAGCAGAGCTCGTTT
AGTGAACCGTCAGATCGCCTGGAGACGCCATCCACGCTGTTTTGACCTCCATAGAAGAC
ACCGACTCTAGAGGATCGATCCCCCGGGCTGCAGGAATTCAAGCGAGAAGACAAGGGC
AGAAAG CACC
51. SEQ ID NO: 51-Transgene mCherry
GTGAGCAAGGGCGAGGAGGATAACATGGCCATCATCAAGGAGTTCATGCGCTTCAAGG
TGCACATGGAGGGCTCCGTGAACGGCCACGAGTTCGAGATCGAGGGCGAGGGCGAGG
GCCGCCCCTACGAGGGCACCCAGACCGCCAAGCTGAAGGTGACCAAGGGTGGCCCCCT
GCCCTTCGCCTGGGACATCCTGTCCCCTCAGTTCATGTACGGCTCCAAGGCCTACGTGA
AGCACCCCGCCGACATCCCCGACTACTTGAAGCTGTCCTTCCCCGAGGGCTTCAAGTGG
GAGCGCGTGATGAACTTCGAGGACGGCGGCGTGGTGACCGTGACCCAGGACTCCTCCC
TGCAGGACGGCGAGTTCATCTACAAGGTGAAGCTGCGCGGCACCAACTTCCCCTCCGA
CGGCCCCGTAATGCAGAAGAAGACCATGGGCTGGGAGGCCTCCTCCGAGCGGATGTAC
CCCGAGGACGGCGCCCTGAAGGGCGAGATCAAGCAGAGGCTGAAGCTGAAGGACGGC
GGCCACTACGACGCTGAGGTCAAGACCACCTACAAGGCCAAGAAGCCCGTGCAGCTGC
CCGGCGCCTACAACGTCAACATCAAGTTGGACATCACCTCCCACAACGAGGACTACAC
CATCGTGGAACAGTACGAACGCGCCGAGGGCCGCCACTCCACCGGCGGCATGGACGAG
CTGTACAAGTAA
52. SEQ ID NO: 52-CD33CAR-Gen2
ATGCTGCTGCTGGTGACCTCTCTGCTGCTGTGCGAGCTGCCACACCCAGCCTTCCTGCT
GATCCCAGACATCCAGATGACACAGAGCCCCAGCTCCCTGAGCGCCTCCGTGGGCGAC
AGAGTGACCATCACATGTAGGGCCTCTGAGAGCGTGGATAACTATGGCATCAGCTTCA
TGAATTGGTTTCAGCAGAAGCCTGGCGGCGCCCCAAAGCTGCTGATCTACGCAGCCAG
CATGCAGGGCTCCGGCGTGCCCTCTCGGTTCTCCGGCTCTGGCAGCGGCACCGACTTCA
CCCTGACAATCTCTAGCCTGCAGCCAGACGATTTCGCCACATACTATTGCCAGCAGAGC
AAGGAGGTGCCCTGGACCTTTGGCCAGGGCACAAAGGTGGAGATCAAGGGCTCCACCT
CTGGCAGCGGCAAGCCTGGCAGCGGAGAGGGCTCCACAAAGGGACAGGTGCAGCTGG
TGCAGTCCGGAGCCGAGGTGAAGAAGCCAGGCTCCTCTGTGAAGGTGTCTTGTAAGGC
CAGCGGCTATACCTTCACAGACTACAACATGCACTGGGTGCGCCAGGCACCAGGACAG
GGCCTGGAGTGGATCGGCTACATCTATCCTTACAACGGCGGCACCGGCTATAATCAGA
AGTTTAAGTCCAAGGCCACCATCACAGCCGATGAGTCTACCAATACAGCCTACATGGA
GCTGAGCAGCCTGCGGTCCGAGGACACAGCCGTGTACTATTGCGCCCGGGGCAGACCC
GCTATGGACTATTGGGGCCAGGGCACCCTGGTGACAGTGTCTAGCGAGAGCAAGTACG
GACCACCTTGCCCACCATGTCCTGCACCAGAGTTCCTGGGAGGACCTTCCGTGTTCCTG
TTTCCTCCAAAGCCAAAGGACACCCTGATGATCAGCCGGACCCCAGAGGTGACATGCG
TGGTGGTGGACGTGAGCCAGGAGGACCCCGAGGTGCAGTTCAACTGGTACGTGGATGG
CGTGGAGGTGCACAATGCCAAGACCAAGCCAAGAGAGGAGCAGTTTAACTCCACCTAT
AGGGTGGTGTCTGTGCTGACAGTGCTGCACCAGGACTGGCTGAACGGCAAGGAGTACA
AGTGCAAGGTGTCCAATAAGGGCCTGCCTTCCTCTATCGAGAAGACCATCTCTAAGGCA
AAGGGACAGCCAAGGGAGCCACAGGTGTATACACTGCCCCCTAGCCAGGAGGAGATG
ACCAAGAACCAGGTGTCCCTGACATGTCTGGTGAAGGGCTTTTACCCTTCTGACATCGC
CGTGGAGTGGGAGAGCAATGGCCAGCCAGAGAACAATTATAAGACCACACCACCCGTG
CTGGACTCTGATGGCAGCTTCTTTCTGTACAGCCGCCTGACCGTGGATAAGTCCCGGTG
GCAGGAGGGCAACGTGTTCTCCTGCTCTGTGATGCACGAGGCCCTGCACAATCACTACA
CACAGAAGAGCCTGTCCCTGTCTCTGGGCAAGATGTTTTGGGTGCTGGTGGTGGTGGGA
GGCGTGCTGGCCTGTTATTCCCTGCTGGTGACCGTGGCCTTCATCATCTTTTGGGTGCGC
TCCAAGCGGAGCCGGGGCGGACACTCTGACTACATGAACATGACCCCACGGAGACCCG
GACCTACAAGGAAGCACTATCAGCCCTACGCCCCTCCACGGGACTTCGCAGCATATCG
CAGCCGGGTGAAGTTTAGCAGATCCGCCGATGCACCAGCATATCAGCAGGGACAGAAT
CAGCTGTACAACGAGCTGAATCTGGGCAGGCGCGAGGAGTACGACGTGCTGGATAAGA
GGCGGGGCCGGGACCCCGAGATGGGAGGCAAGCCCAGGCGCAAGAACCCTCAGGAGG
GCCTGTATAATGAGCTGCAGAAGGACAAGATGGCCGAGGCCTACAGCGAGATCGGCAT
GAAGGGAGAGCGGAGAAGGGGCAAGGGACACGATGGCCTGTATCAGGGCCTGTCCAC
CGCCACAAAGGACACCTACGATGCACTGCACATGCAGGCCCTGCCACCTCGGTGA
53. SEQ ID NO: 53-CD33CAR-Gen4v2
ATGCTGCTGCTGGTGACCTCCCTGCTGCTGTGCGAGCTGCCACACCCTGCCTTTCTGCTG
ATCCCAGACATCCAGATGACACAGAGCCCCAGCTCCCTGTCTGCCAGCGTGGGCGACA
GAGTGACCATCACATGTAGGGCCTCCGAGTCTGTGGATAACTATGGCATCAGCTTTATG
AATTGGTTCCAGCAGAAGCCAGGAGGCGCCCCTAAGCTGCTGATCTACGCAGCCTCCA
TGCAGGGCTCTGGCGTGCCCAGCCGCTTTAGCGGCTCCGGCTCTGGCACCGATTTCACC
CTGACAATCTCTAGCCTGCAGCCAGACGATTTTGCCACATACTATTGCCAGCAGTCCAA
GGAGGTGCCCTGGACCTTCGGCCAGGGCACAAAGGTGGAGATCAAGGGCAGCACCTCC
GGCTCTGGCAAGCCTGGCTCCGGAGAGGGCTCTACAAAGGGACAGGTGCAGCTGGTGC
AGAGCGGAGCCGAGGTGAAGAAGCCAGGCTCCTCTGTGAAGGTGAGCTGTAAGGCCTC
CGGCTATACCTTTACAGACTACAACATGCACTGGGTGAGACAGGCACCAGGACAGGGC
CTGGAGTGGATCGGCTACATCTATCCTTACAACGGCGGCACCGGCTATAATCAGAAGTT
CAAGAGCAAGGCCACCATCACAGCCGATGAGTCCACCAATACAGCCTACATGGAGCTG
AGCAGCCTGAGGAGCGAGGACACAGCCGTGTACTATTGCGCCAGAGGCAGGCCTGCTA
TGGACTATTGGGGCCAGGGCACCCTGGTGACAGTGTCTAGCGAGTCCAAGTACGGACC
ACCTTGCCCACCATGTCCAGCACCAGAGTTTCTGGGAGGACCTAGCGTGTTTCTGTTCC
CTCCAAAGCCAAAGGACACCCTGATGATCAGCAGAACCCCCGAGGTGACATGCGTGGT
GGTGGACGTGTCCCAGGAGGACCCCGAGGTGCAGTTTAACTGGTACGTGGATGGCGTG
GAGGTGCACAATGCCAAGACCAAGCCTAGAGAGGAGCAGTTCAACTCCACCTATAGGG
TGGTGTCTGTGCTGACAGTGCTGCACCAGGACTGGCTGAACGGCAAGGAGTACAAGTG
CAAGGTGTCTAATAAGGGCCTGCCATCCTCTATCGAGAAGACCATCAGCAAGGCCAAG
GGCCAGCCTAGGGAGCCACAGGTGTATACACTGCCCCCTTCCCAGGAGGAGATGACCA
AGAACCAGGTGTCTCTGACATGTCTGGTGAAGGGCTTCTACCCATCCGACATCGCCGTG
GAGTGGGAGTCTAATGGCCAGCCCGAGAACAATTATAAGACCACACCACCCGTGCTGG
ACTCTGATGGCAGCTTCTTTCTGTACTCTCGCCTGACCGTGGATAAGAGCCGGTGGCAG
GAGGGCAACGTGTTTAGCTGCTCCGTGATGCACGAGGCCCTGCACAATCACTACACAC
AGAAGTCTCTGAGCCTGTCCCTGGGCAAGAGCAACCTGTTCGTGGCCTCCTGGATCGCC
GTGATGATCATCTTTCGCATCGGCATGGCCGTGGCCATCTTCTGCTGTTTCTTTTTCCCA
TCCGGAGGCTCTGGAGGAGGCTCCGGCTGGCGGAGAAAGCGGAAGGAGAAGCAGAGC
GAGACCTCCCCTAAGGAGTTTCTGACAATCTATGAGGACGTGAAGGATCTGAAGACCA
GGCGCAATCACGAGCAGGAGCAGACCTTCCCAGGAGGAGGCTCTACAATCTACAGCAT
GATCCAGTCCCAGAGCAGCGCCCCAACCAGCCAGGAGCCAGCCTATACACTGTACTCT
CTGATCCAGCCTAGCCGGAAGTCTGGCAGCCGCAAGCGGAACCACTCCCCATCTTTCAA
TTCTACCATCTATGAAGTGATCGGCAAGAGCCAGCCTAAGGCCCAGAACCCAGCCAGA
CTGTCCAGGAAGGAGCTGGAGAATTTTGACGTGTACTCTGGAGGCAGCGGAGGAGGCT
CTGGCCGCGTGAAGTTCAGCCGGTCCGCCGATGCCCCAGCCTATAAGCAGGGCCAGAA
CCAGCTGTACAACGAGCTGAATCTGGGCCGGAGAGAGGAGTACGACGTGCTGGATAAG
AGGCGGGGCCGGGACCCCGAGATGGGAGGCAAGCCCCGGAGAAAGAACCCTCAGGAG
GGCCTGTATAATGAGCTGCAGAAGGACAAGATGGCCGAGGCCTACTCCGAGATCGGCA
TGAAGGGAGAGAGGCGCCGGGGCAAGGGACACGATGGCCTGTATCAGGGCCTGAGCA
CCGCCACAAAGGACACCTACGATGCCCTGCACATGCAGGCCCTGCCTCCACGGTGATG
A
54. SEQ ID NO: 54-BGHpA
CCTCGACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGC
ATTGTCTGAGTAGGTGTCATTCTATTC
55. SEQ ID NO: 55-1000 bp left Homology arm
GATTGGTGACAGAAAAGCCCCATCCTTAGGCCTCCTCCTTCCTAGTCTCCTGATATTGG
GTCTAACCCCCACCTCCTGTTAGGCAGATTCCTTATCTGGTGACACACCCCCATTTCCTG
GAGCCATCTCTCTCCTTGCCAGAACCTCTAAGGTTTGCTTACGATGGAGCCAGAGAGGA
TCCTGGGAGGGAGAGCTTGGCAGGGGGTGGGAGGGAAGGGGGGGATGCGTGACCTGC
CCGGTTCTCAGTGGCCACCCTGCGCTACCCTCTCCCAGAACCTGAGCTGCTCTGACGCG
GCTGTCTGGTGCGTTTCACTGATCCTGGTGCTGCAGCTTCCTTACACTTCCCAAGAGGA
GAAGCAGTTTGGAAAAACAAAATCAGAATAAGTTGGTCCTGAGTTCTAACTTTGGCTCT
TCACCTTTCTAGTCCCCAATTTATATTGTTCCTCCGTGCGTCAGTTTTACCTGTGAGATA
AGGCCAGTAGCCAGCCCCGTCCTGGCAGGGCTGTGGTGAGGAGGGGGGTGTCCGTGTG
GAAAACTCCCTTTGTGAGAATGGTGCGTCCTAGGTGTTCACCAGGTCGTGGCCGCCTCT
ACTCCCTTTCTCTTTCTCCATCCTTCTTTCCTTAAAGAGTCCCCAGTGCTATCTGGGACAT
ATTCCTCCGCCCAGAGCAGGGTCCCGCTTCCCTAAGGCCCTGCTCTGGGCTTCTGGGTT
TGAGTCCTTGGCAAGCCCAGGAGAGGCGCTCAGGCTTCCCTGTCCCCCTTCCTCGTCCA
CCATCTCATGCCCCTGGCTCTCCTGCCCCTTCCCTACAGGGGTTCCTGGCTCTGCTCTTC
AGACTGAGCCCCGTTCCCCTGCATCCCCGTTCCCCTGCATCCCCCTTCCCCTGCATCCCC
CAGAGGCCCCAGGCCACCTACTTGGCCTGGACCCCACGAGAGGCCACCCCAGCCCTGT
CTACCAGGCTGCCTTTTGGGTGGATTCTCCTCCAACTGTGGGGTGACTGCTTGG
56. SEQ ID NO: 56-Vector: 149 (head) fused to Vector: 4116 (tail) (in
bold) with 9 homologous bases (in italics)
GGCATGGGGTTGGGTGAGGGAGGAGAGATGCCCGGAGAGGACCCAGACACGGGGAGG
ATCCGCTCAGAGGACATCACGTGGTGCAGCGGCGCGCCGGCCGCAGAAAGGGAGTAG
AGGCGGCCACGACCTGGTGAACACCTAGGACGCACCATTCTCACAAAGGGAGTTT
TCCACACGGACACCCCCCTCCTCACCACAGCCCTGCCAGGACGGGGCTGGCTACT
GGCCTTATCTC
57. SEQ ID NO: 57-Vector: 149 (head) fused to Vector: 4,113 (tail) (in
bold) with 12 homologous bases (in italics
GCGAGTGAAGACGGCATGGGGTTGGGTGAGGGAGGAGAGATGCCCGGAGAGGACCCA
GACACGGGGAGGATCCGCTCAGAGGACATCACGTGGTGCAGCGGCGCGCCGGCCGCAG
GAAGGGAGTAGAGGCGGCCACGACCTGGTGAACACCTAGGACGCACCATTCTCAC
AAAGGGAGTTTTCCACACGGACACCCCCCTCCTCACCACAGCCCTGCCAGGACGG
GGCTGGCTACTGGCCTTA
58. SEQ ID NO: 58-Vector: 155 (head) fused to Vector: 4,163 (tail) (in
bold) with 6 homologous bases (in italics)
GCGAGTGAAGACGGCATGGGGTTGGGTGAGGGAGGAGAGATGCCCGGAGAGGACCCA
GACACGGGGAGGATCCGCTCAGAGGACATCACGTGGTGCAGCGGCGCGCAGAGAGGG
AGTGGCCAACTCCATCACTAGGGGTTCCTGCGGCCGCAGAAAGGGAGTAGAGGCG
GCCACGACCTGGTGAACACCTAGGACGCACCATTCTCACAAAGGGAGTTTTCCACA
CGGA
59. SEQ ID NO: 59-Vector: 158 (head) fused to Vector: 4,121 (tail) (in
bold) with 4 homologous bases (in italics)
GCGAGTGAAGACGGCATGGGGTTGGGTGAGGGAGGAGAGATGCCCGGAGAGGACCCA
GACACGGGGAGGATCCGCTCAGAGGACATCACGTGGTGCAGCGGCCGCAGAAAGGGA
GTAGAGGCGGCCACGACCTGGTGAACACCTAGGACGCACCATTCTCACAAAGGGA
GTTTTCCACACGGACACCCCCCTCCTCACCACAGCCCTGCCAGGACGGGGCTGGC
TACTGGCCTT

1. A composition comprising a single-chain variable fragment (scFv) comprising a heavy chain variable region (VH), a light chain variable region (VL), and an acceptable carrier, wherein the VH comprises one, two, or three heavy chain complementarity determining regions (CDRHs), and the VL comprises one, two, or three light chain complementarity determining regions (CDRLs), and wherein the CDRHs or the CDRLs comprise at least 80% sequence identity to SEQ ID NO: 16, SEQ ID NO: 17, SEQ ID NO: 18, SEQ ID NO: 19, SEQ ID NO: 20, or SEQ ID NO: 21.

2. The composition of claim 1, wherein the scFv comprises at least 70% sequence identity to SEQ ID NO: 52, SEQ ID NO: 53, or a variant thereof.

3. The composition of claim 1, wherein the VH comprises at least 70% sequence identity to SEQ ID NO: 1, SEQ ID NO: 2, SEQ ID NO: 3, SEQ ID NO: 4, SEQ ID NO: 5 or SEQ ID NO: 6, and the VL comprises at least 60% sequence identity to SEQ ID NO: 7, SEQ ID NO: 8, SEQ ID NO: 9, SEQ ID NO: 10, SEQ ID NO: 11 or SEQ ID NO: 12.

4. The composition of claim 1, further comprising a linker attached to the VH and the VL, wherein the linker comprises at least 80% sequence identity to SEQ ID NO: 13.

5. The composition of claim 1, further comprising at least 80% sequence identity to SEQ ID NO: 23, SEQ ID NO: 24, SEQ ID NO: 25, SEQ ID NO: 26, SEQ ID NO: 27, or SEQ ID NO: 28.

6. The composition of claim 1, wherein the composition comprises a chimeric antigen receptor (CAR).

7. A nucleic acid encoding the scFV claim 1.

8. An expression vector comprising a nucleic acid encoding the scFv of claim 1.

9. A cell comprising the composition of claim 1.

10. The cell of claim 9, comprising a T-cell, a natural killer (NK) cell, a genetically-modified T-cell, or a genetically-modified NK cell.

11. A method of treating a CD33-associated pathology in a subject in need thereof, the method comprising administering to the subject a therapeutically effective amount of a composition comprising a single-chain variable fragment (scFv), wherein the scFv comprises a heavy chain variable region (VH), a light chain variable region (VL), and an acceptable carrier, wherein the VH comprises one, two, or three heavy chain complementarity determining regions (CDRHs), and the VL comprises one, two, or three light chain complementarity determining regions (CDRLs), and wherein the CDRHs or the CDRLs comprise at least 80% sequence identity to SEQ ID NO: 16, SEQ ID NO: 17, SEQ ID NO: 18, SEQ ID NO: 19, SEQ ID NO: 20, or SEQ ID NO: 21.

12. The method of claim 11, wherein the scFv comprises at least 70% sequence identity to SEQ ID NO: 52, SEQ ID NO: 53, or a variant thereof.

13. The method of claim 11, wherein the VH comprises at least 70% sequence identity to SEQ ID NO: 1, SEQ ID NO: 2, SEQ ID NO: 3, SEQ ID NO: 4, SEQ ID NO: 5 or SEQ ID NO: 6, and the VL comprises at least 60% sequence identity to SEQ ID NO: 7, SEQ ID NO: 8, SEQ ID NO: 9, SEQ ID NO: 10, SEQ ID NO: 11 or SEQ ID NO: 12.

14. The method of claim 11, further comprising a linker attached to the VH and the VL, wherein the linker comprises at least 80% sequence identity to SEQ ID NO: 13.

15. The method of claim 11, further comprising at least 80% sequence identity to SEQ ID NO: 23, SEQ ID NO: 24, SEQ ID NO: 25, SEQ ID NO: 26, SEQ ID NO: 27, or SEQ ID NO: 28.

16. The method of claim 11, wherein the composition comprises a chimeric antigen receptor (CAR).

17. The method of claim 11, wherein the method treats a hematologic cancer, neurodegenerative disorder, lymphoma, leukemia, systemic lupus erythematosus, rheumatoid arthritis, asthma, allergy, sepsis, chronic obstructive pulmonary disease, graft-versus-host disease (GVHD), eosinophilia, or osteoporosis.

18. The method of claim 11, wherein the hematologic cancer is acute myelogenous leukemia.

19. The method of claim 11, wherein the neurodegenerative disorder is Alzheimer's.
